SEQUENCE LISTING (946 sequences)

SEQ ID NO: 1-50 - skipped (50 entries)

SEQ ID NO: 51 - protein, 5 aa, Artificial Sequence
  synthetic
GNSRV

SEQ ID NO: 52 - protein, 6 aa, Artificial Sequence
  Synthetic
ARGVDE

SEQ ID NO: 53-81 - skipped (29 entries)

SEQ ID NO: 82 - protein, 6 aa, Artificial Sequence
  Synthetic
EDVGRA

SEQ ID NO: 83 - protein, 5 aa, Artificial Sequence
  Synthetic
GGGGG

SEQ ID NO: 84 - protein, 5 aa, Artificial Sequence
  Synthetic
AGGGG

SEQ ID NO: 85 - protein, 5 aa, Artificial Sequence
  Synthetic
VGGGG

SEQ ID NO: 86 - protein, 5 aa, Artificial Sequence
  Synthetic
FGGGG

SEQ ID NO: 87 - protein, 5 aa, Artificial Sequence
  Synthetic
NGGGG

SEQ ID NO: 88 - protein, 5 aa, Artificial Sequence
  Synthetic
SGGGG

SEQ ID NO: 89 - protein, 5 aa, Artificial Sequence
  Synthetic
RGGGG

SEQ ID NO: 90 - protein, 5 aa, Artificial Sequence
  Synthetic
DGGGG

SEQ ID NO: 91-92 - skipped (2 entries)

SEQ ID NO: 93 - protein, 5 aa, Artificial Sequence
  Synthetic
SNNGR

SEQ ID NO: 94-155 - skipped (62 entries)

SEQ ID NO: 156 - protein, 5 aa, Artificial Sequence
  Synthetic
DVRRV

SEQ ID NO: 157-166 - skipped (10 entries)

SEQ ID NO: 167 - protein, 5 aa, Artificial Sequence
  Synthetic
DVRRV

SEQ ID NO: 168 - protein, 5 aa, Artificial Sequence
  Synthetic
FGSAF

SEQ ID NO: 169-225 - skipped (57 entries)

SEQ ID NO: 226 - protein, 5 aa, Artificial Sequence
  Synthetic
NFAVA

SEQ ID NO: 227 - protein, 5 aa, Artificial Sequence
  Synthetic
SVFVR

SEQ ID NO: 228-283 - skipped (56 entries)

SEQ ID NO: 284 - protein, 5 aa, Artificial Sequence
  Synthetic
SFGGF

SEQ ID NO: 285-301 - skipped (17 entries)

SEQ ID NO: 302 - protein, 5 aa, Artificial Sequence
  Synthetic
RNANS

SEQ ID NO: 303-341 - skipped (39 entries)

SEQ ID NO: 342 - protein, 5 aa, Artificial Sequence
  Synthetic
GFFAF

SEQ ID NO: 343-366 - skipped (24 entries)

SEQ ID NO: 367 - protein, 5 aa, Artificial Sequence
  Synthetic
DNVAD

SEQ ID NO: 368 - protein, 5 aa, Artificial Sequence
  Synthetic
AADAN

SEQ ID NO: 369-397 - skipped (29 entries)

SEQ ID NO: 398 - protein, 5 aa, Artificial Sequence
  Synthetic
VGRVS

SEQ ID NO: 399-437 - skipped (39 entries)

SEQ ID NO: 438 - protein, 5 aa, Artificial Sequence
  Synthetic
FVGNA

SEQ ID NO: 439-502 - skipped (64 entries)

SEQ ID NO: 503 - protein, 5 aa, Artificial Sequence
  Synthetic
GFNVV

SEQ ID NO: 504-547 - skipped (44 entries)

SEQ ID NO: 548 - protein, 5 aa, Artificial Sequence
  Synthetic
FVVNG

SEQ ID NO: 549-560 - skipped (12 entries)

SEQ ID NO: 561 - protein, 5 aa, Artificial Sequence
  Synthetic
VDGDA

SEQ ID NO: 562-563 - skipped (2 entries)

SEQ ID NO: 564 - protein, 5 aa, Artificial Sequence
  Synthetic
VGSDD

SEQ ID NO: 565-582 - skipped (18 entries)

SEQ ID NO: 583 - protein, 5 aa, Artificial Sequence
  Synthetic
NADRV

SEQ ID NO: 584 - skipped

SEQ ID NO: 585 - protein, 5 aa, Artificial Sequence
  Synthetic
VVSFV

SEQ ID NO: 586-647 - skipped (62 entries)

SEQ ID NO: 648 - protein, 5 aa, Artificial Sequence
  Synthetic
GAVNF

SEQ ID NO: 649-690 - skipped (42 entries)

SEQ ID NO: 691 - protein, 5 aa, Artificial Sequence
  Synthetic
DGRNS

SEQ ID NO: 692 - skipped

SEQ ID NO: 693 - protein, 5 aa, Artificial Sequence
  Synthetic
VFARG

SEQ ID NO: 694-719 - skipped (26 entries)

SEQ ID NO: 720 - protein, 5 aa, Artificial Sequence
  Synthetic
DFRVF

SEQ ID NO: 721-754 - skipped (34 entries)

SEQ ID NO: 755 - protein, 5 aa, Artificial Sequence
  Synthetic
NGGRA

SEQ ID NO: 756-789 - skipped (34 entries)

SEQ ID NO: 790 - protein, 5 aa, Artificial Sequence
  Synthetic
FFDDS

SEQ ID NO: 791-811 - skipped (21 entries)

SEQ ID NO: 812 - protein, 5 aa, Artificial Sequence
  Synthetic
AHCVE

SEQ ID NO: 813-846 - skipped (34 entries)

SEQ ID NO: 847 - protein, 5 aa, Artificial Sequence
  Synthetic
QWRNH

SEQ ID NO: 848-880 - skipped (33 entries)

SEQ ID NO: 881 - protein, 5 aa, Artificial Sequence
  Synthetic
AHCVE

SEQ ID NO: 882-904 - skipped (23 entries)

SEQ ID NO: 905 - protein, 5 aa, Artificial Sequence
  Synthetic
QWRNH

SEQ ID NO: 906-945 - skipped (40 entries)

SEQ ID NO: 946 - protein, 6 aa, Artificial Sequence
  Synthetic
RGVDEA

(19) United States
(12) Patent Application Publication
(10) Pub. No.: US 2025/0372195 A1
(43) Pub. Date: Dec. 4, 2025

(54) CYCLIC PEPTIDE STRUCTURE PREDICTION VIA STRUCTURAL ENSEMBLES ACHIEVED BY MOLECULAR DYNAMICS AND MACHINE LEARNING

(71) Applicant: Trustees of Tufts College, Medford, MA (US)

(72) Inventors: Jiayuan Miao, Medford, MA (US); Yu-Shan Lin, Medford, MA (US)

(21) Appl. No.: 18/570,394

(22) PCT Filed: Jun. 14, 2022
(86) PCT No.: PCT/US22/72941

§ 371 Date: Dec. 14, 2023

Related U.S. Application Data
(60) Provisional application No. 63/202,488, filed on Jun. 14, 2021.
(60) Provisional application No. 63/255,837, filed on Oct. 14, 2021.

(51) Int. Cl. G16B 15/20; G16B 15/30; G16B 40/20

(52) U.S. Cl. CPC G16B 15/20; G16B 15/30; G16B 40/20

(57) ABSTRACT

Disclosed herein are methods and systems for using molecular dynamics simulation results as training datasets for machine-learning models that can provide predictions of cyclic peptide structural ensembles.

### CROSS REFERENCE TO RELATED APPLICATIONS

This application is the national stage entry of PCT/US2022/072941, filed Jun. 14, 2022, which is based on, and claims benefit of priority to U.S. Patent Application No. 63/255,837, filed Oct. 14, 2021, and U.S. Patent Application No. 63/202,488, filed Jun. 14, 2021. The contents of each are incorporated by reference in their entirety.

### STATEMENT REGARDING FEDERALLY SPONSORED RESEARCH

This invention was made with government support under R01GM124160 awarded by the National Institutes of Health. The government has certain rights in the invention.

### SEQUENCE LISTING

This application is being filed electronically and includes an electronically submitted Sequence Listing in .txt format. The .txt file contains a sequence listing entitled “16611801372_ST25.txt” created on Apr. 14, 2025 and is 36,417 bytes in size. The Sequence Listing contained in this .txt file is part of the specification and is hereby incorporated by reference herein in its entirety.

### BACKGROUND

Computational methods have made strides in discovering well-structured cyclic peptides that preferentially populate a single conformation. However, many successful cyclic-peptide therapeutics adopt multiple conformations in solutions. In fact, the chameleonic properties of some cyclic peptides are likely responsible for their high cell membrane permeability. Thus, we require the ability to predict complete structural ensembles for cyclic peptides, including the majority of cyclic peptides that have broad structural ensembles, to significantly improve our ability to rationally design cyclic-peptide therapeutics. As a result, there is a need for new methods for cyclic peptide structure prediction.

### SUMMARY OF THE INVENTION

Disclosed herein are methods and systems for using molecular dynamics simulation results as training datasets for machine-learning models that can provide predictions of cyclic peptide structural ensembles.

One aspect of the invention provides for a method for predicting a structure of a cyclic peptide, the method comprising providing a weight vector w, wherein w comprises a multiplicity residue weights of an adopted structure and a multiplicity of partition function weights, providing a coefficient matrix A configured to select which of the multiplicity residue weights of the adopted structure and which one of the multiplicity of partition function weights are used to determine the population of a cyclic peptide adopting the structure, and determining the population of the structure of the cyclic peptide from the multiplicity of residue weights and multiplicity of partition function weights. The multiplicity of residue weights of the adopted structure and the multiplicity of partition function weights are determined by minimizing the difference between a predicted population and an actual population observed in a training dataset.

In some embodiments, the multiplicity of residue weights are a multiplicity of pairwise residue weights, e.g., (1, 2) residue weights, (1, 3) residue weights, (1,) residue weights, or any combination thereof. The training dataset may be obtained from molecular dynamics simulation.

Another aspect of the invention provides for a method for predicting a structure of a cyclic peptide, the method comprising encoding the cyclic peptide, and determining a population of the structure of the cyclic peptide. In some embodiments, the cyclic peptide is encoded with a molecular fingerprint encoding scheme. In some embodiments, the method further comprises representing a cyclic peptide as a graph with a node for every amino acid of the cyclic peptide and connecting a node pair by forward and backward edges, e.g., (1, 2) neighbor node pairs, (1, 3) neighbor node pairs, (1, 4) neighbor node pairs, or any combination thereof. In some embodiments, the initial node representation is given by an amino acid molecular fingerprint. The neural network for determining the structure may be a graph neural network. In some embodiments, the method further comprises arranging an initial representation of the cyclic peptide such that neighboring amino acids have features adjacent in space. The neural network for determining the structure may be a convolutional neural network. The neural network may be trained with a training dataset obtained from a molecular dynamics simulation.

In some embodiments, the methods described herein may be used to select a cyclic peptide. The method may comprise performing any of the methods for predicting the structure of a cyclic peptide described herein and selecting well-structured cyclic peptides. In some embodiments, the method further comprises synthesizing a selected cyclic peptide and, optionally, assaying the synthesized cyclic peptide. In other embodiments, the cyclic peptide for assay.

Another aspect of the invention provides for a computation platform comprising a communication interface that receives cyclic peptide information, and a computer in communication with the communication interface, wherein the computer comprises a computer processor and a computer readable medium comprising machine-executable code that, upon execution by the computer processor, implements any of the methods for predicting the structure of a cyclic peptide described herein.

Another aspect of the invention provides for computer readable medium comprising machine-executable code that, upon execution by the computer processor, implements any of the methods for predicting a cyclic peptide described herein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1A provides a flowchart of an exemplary structure prediction methodology.

FIG. 1B provides a flowchart of an exemplary structure prediction methodology.

FIG. 1C. The Structural Ensembles Achieved by Molecular Dynamics and Machine Learning (StrEAMM) method integrates molecular dynamics (MD) simulation and machine learning to enable efficient prediction of cyclic peptide structural ensembles. Using MD simulation results as the training dataset, a StrEAMM model was built that quickly predicted structural ensembles of cyclic peptides of new sequences for both well- and non-well-structured cyclic peptides. In the cyclic peptide sequences shown on the left, lowercase letters denote D-amino acids. In the two example structural ensembles given on the right, cyclo-(avVrr) is considered well-structured with the population of the most-populated structure being >50%; on the other hand, cyclo-(SVFAa) is non-well-structured with no conformation whose population is >50%.

FIG. 2. Extant scoring function and new StrEAMM models. a, Scoring Function 1.0. This version of the scoring function is similar to the one developed by Slough et al., 24 which for a cyclic pentapeptide cyclo-(X₁X₂X₃X₄X₅) uses 5 parent sequences cyclo-(X₁X₂GGG), cyclo-(GX₂X₃GG), cyclo-(GGX₃X₄G), cyclo-(GGGX₄X₅), and cyclo-(X₁GGGX₅), to capture the effects from the 5 nearest-neighbor pairs and sums the populations observed in the MD simulations of the 5 parent sequences to build the final score. b, StrEAMM model (1,2)/sys. This model considers the effects of the nearest-neighbor pairs as effective weights. The logarithm of the population of a structure can be expressed by the summation of the 5 weights and the weight related to the partition function. c. StrEAMM models (1,2)+(1,3)/sys and (1,2)+(1,3)/random. These models consider interactions between both the nearest-neighbor and next-nearest-neighbor residues, i.e., both (1, 2) and (1, 3) interactions. The logarithm of the population of a structure can be expressed by the summation of the 10 weights and the weight related to the partition function. R groups of amino acids are represented by spheres. Different colors stand for different structural digits.

FIG. 3. The comparison between scores predicted by Scoring Function 1.0 and the actual populations of various structures observed in the MD simulations of 50 random sequences in the test dataset (Dataset 4). Only structures whose observed populations in MD simulations are above 1% or whose predicted scores are above 0.01 are shown. Scoring Function 1.0 successfully predicts the most-populated structures of 11 out of the 50 cyclic peptides in the test datasets and these 11 structures are shown as orange stars. There is a poor correlation between the observed populations in MD simulations and the predicted scores (highlighted by red circles).

FIG. 4. Comparison of performance of Scoring Function 1.0 and the StrEAMM Models on two specific cyclic peptides. a, Cyclo-(avVrr), a well-structured cyclic peptide with the population of the most-populated structure being >50% (58.6%). b, Cyclo-(SVFAa), a non-well-structured cyclic peptide that adopts multiple conformations with small populations. For each cyclic peptide, the three most-populated structures are shown, with a representative conformation shown in sticks and 100 randomly selected conformations shown in magenta lines. The actual populations observed in the MD simulations of the two cyclic peptides are given and compared to the predictions made by Scoring Function 1.0 and StrEAMM Models (1,2)/sys, (1,2)+(1,3)/sys, and (1,2)+(1,3)/random.

FIG. 5. Weighted least square fitting results for the training dataset (top row) and the performance on the test dataset (bottom row) of the three StrEAMM models. a and b, StrEAMM Model (1,2)/sys. c and d, StrEAMM Model (1,2)+(1,3)/sys. e and f, StrEAMM Model (1,2)+(1,3)/random. Top row: Comparison between the fitted populations and the actual populations of various structures observed in the MD simulations of the training dataset. Bottom row: Comparison between the populations predicted by each StrEAMM model and the actual populations of various structures observed in the MD simulations of 50 random test sequences; only structures with observed populations or predicted populations >1% are shown. Predicted populations in b, d and f were calculated by Eq. (3), (11), and (11), respectively. Pearson correlation coefficient (R), weighted error

$$( W E = \frac{{\sum }_{i} {p}_{i , observed} \vert {p}_{i , observed} - {p}_{i , theory} \vert}{{\sum }_{i} {p}_{i , observed}} ,$$

where p_{i,theory} is the fitted population or the predicted population), and weighted squared error

$$( W S E = \frac{{\sum }_{i} {{p}_{i , observed} ( {p}_{i , observed} - {p}_{i , theory} )}^{2}}{{\sum }_{i} {p}_{i , observed}} )$$

were calculated. Gray lines show where the fitted/predicted populations equal to the observed populations. StrEAMM Models (1,2)/sys, (1,2)+(1,3)/sys, and (1,2)+(1,3)/random successfully predict the most-populated structures of 12, 30, and 43 out of the 50 cyclic peptides in the test dataset, respectively, and these structures are shown as orange stars in b, d, and f.

FIG. 6. The Ramachandran plot is divided into 10 regions for structural description. a, The total probability distribution of (ϕ, ψ) of the five residues of cyclo-(GGGGG) (SEQ ID NO: 83). b, According to the distribution in a, the (ϕ, ψ) space was discretized into 10 regions: Λ, λ, Γ, γ, B, β, Π, π, Z, and ζ.

FIG. 7. Illustration of the matrix equation (7) In p=Aw. The logarithms of populations (In p) are arranged into a column vector of size N, where N is the summation of the number of structure types of each cyclic peptide in the training set. Different weights (w) are arranged into a column vector of size M, where M is the number of weights. Weights that are mirror images of each other are treated as equal, for example,

$${w}_{{S}_{i} {S}_{i + 1}}^{{X}_{i} {X}_{i + 1}} = {w}_{{s}_{i} {s}_{i + 1}}^{{x}_{i} {x}_{i + 1}} , {w}_{{S}_{i} {S}_{i + 1} {S}_{i + 2}}^{{X}_{i} {\_ X}_{i + 2}} = {w}_{{s}_{i} {s}_{i + 1} {s}_{i + 2}}^{{x}_{i -} {x}_{i + 2}} , and {w}_{Q}^{{X}_{1} {X}_{2} {X}_{3} {X}_{4} {X}_{5}} = {w}_{Q}^{{x}_{1} {x}_{2} {x}_{3} {x}_{4} {x}_{5}} ,$$

with capital and lowercase letter pairs representing enantiomers of amino acids and structures. The coefficient matrix A controls which weights are used to compute the population of a specific cyclic-peptide sequence adopting a specific structure.

FIG. 8. Performance of Scoring Function 1.0 on the test Dataset 4. Subplots show comparison between scores predicted by Scoring Function 1.0 and the actual populations of various structures observed in the MD simulations for 50 random sequences. Only structures whose observed populations are above 1% or whose predicted scores are above 0.01 are shown. Green boxes show cyclic peptides whose top structures were predicted correctly by the scoring function.

FIG. 9. Distribution of weights for StrEAMM Model (1,2)/sys. The weights are related to (1, 2) interactions. Both enantiomers of a weight are shown.

FIG. 10. Performance of StrEAMM Model (1,2)/sys on the test Dataset 4. Subplots show comparison between populations predicted by StrEAMM Model (1,2)/sys and the actual populations of various structures observed in the MD simulations for 50 random sequences. Only structures with observed populations or predicted populations >1% are shown. Gray lines show where the predicted populations equal real populations. Green boxes show cyclic peptides whose top structures were predicted correctly by the StrEAMM model.

FIG. 11. Distributions of weights for StrEAMM Model (1,2)+(1,3)/sys. a, Distribution of the weights related to (1, 2) interactions. Both enantiomers of a weight are shown. b, Distribution of the weights related to (1, 3) interactions. Both enantiomers of a weight are shown.

FIG. 12. Performance of StrEAMM Model (1,2)+(1,3)/sys on the test Dataset 4. Subplots show comparison between populations predicted by StrEAMM Model (1,2)+(1,3)/sys and the actual populations of various structures observed in the MD simulations for 50 random sequences. Only structures with observed populations or predicted populations >1% are shown. Gray lines show where the predicted populations equal real populations. Green boxes show cyclic peptides whose top structures were predicted correctly by the StrEAMM model.

FIG. 13. Distributions of weights for StrEAMM Model (1,2)+(1,3)/random. a, Distribution of the weights related to (1, 2) interactions. Both enantiomers of a weight are shown. b, Distribution of the weights related to (1, 3) interactions. Both enantiomers of a weight are shown.

FIG. 14. Performance of StrEAMM Model (1,2)+(1,3)/random on the test Dataset 4. Subplots show comparison between populations is predicted by StrEAMM Model (1,2)+(1,3)/random and the actual populations of various structures observed in the MD simulations for 50 random sequences. Only structures with observed populations or predicted populations >1% are shown. Gray lines show where the predicted populations equal real populations. Green boxes show cyclic peptides whose top structures were predicted correctly by the StrEAMM model.

FIG. 15. Performance of StrEAMM Model (1,2)+(1,3)/sys37. a, Comparison between the fitted populations and the actual populations of various structures observed in the MD simulations of the training dataset. b, Comparison between the populations predicted by StrEAMM model (1,2)+(1,3)/sys37 and the actual populations of various structures observed in the MD simulations of 75 random test sequences (List S4); only structures with observed populations or predicted populations >1% are shown. Pearson correlation coefficient (R), weighted error

$$( W E = \frac{{\sum }_{i} {p}_{i , observed} \vert {p}_{i , observed} - {p}_{i , theory} \vert}{{\sum }_{i} {p}_{i , observed}} ,$$

where p_{i,theory} is the fitted population or the predicted population), and weighted squared error

$$( W S E = \frac{{\sum }_{i} {{p}_{i , observed} ( {p}_{i , observed} - {p}_{i , theory} )}^{2}}{{\sum }_{i} {p}_{i , observed}} )$$

were calculated. Gray lines show where the fitted/predicted populations equal to the observed populations in MD simulations. StrEAMM Model (1,2)+(1,3)/sys37 successfully predicts the most-populated structures of 51 out of the 75 cyclic peptides in the test dataset, and these structures are shown as orange stars.

FIG. 16. Performance of StrEAMM Model GNN/random. a, Comparison between the fitted populations and the actual populations of various structures observed in the MD simulations of the training dataset (Dataset 3). b, Comparison between the populations predicted by StrEAMM model GNN/random and the actual populations of various structures observed in the MD simulations of 50 random test sequences (Dataset 4, List S2); only structures with observed populations or predicted populations >1% are shown. The model successfully predicts the most-populated structures of 42 out of the 50 cyclic peptides in the test dataset, and these structures are shown as orange stars. c, Comparison between the populations predicted by StrEAMM model GNN/random and the actual populations of various structures observed in the MD simulations of another 25 random test sequences including 37 amino acids (Dataset 6.2, List S5); only structures with observed populations or predicted populations >1% are shown. The model successfully predicts the most-populated structures of 13 out of the 25 cyclic peptides in the test dataset, and these structures are shown as orange stars. Pearson correlation coefficient (R), weighted error (WE), and weighted squared error (WSE) were calculated. Gray lines show where the fitted/predicted populations equal to the observed populations in MD simulations.

FIG. 17. Performance of StrEAMM Model GNN/random37. a, Comparison between the fitted populations and the actual populations of various structures observed in the MD simulations of the training dataset (705 sequences in Dataset 3 including 15 amino acids, plus another 50 random sequences in Dataset 6.1 (List S5) including 37 amino acids). b, Comparison between the populations predicted by StrEAMM model GNN/random37 and the actual populations of various structures observed in the MD simulations of 50 random test sequences (Dataset 4, List S2); only structures with observed populations or predicted populations >1% are shown. The model successfully predicts the most-populated structures of 43 out of the 50 cyclic peptides in the test dataset, and these structures are shown as orange stars. c, Comparison between the populations predicted by StrEAMM model GNN/random37 and the actual populations of various structures observed in the MD simulations of another 25 random test sequences including 37 amino acids

(Dataset 6.2, List S5); only structures with observed populations or predicted populations >1% are shown. The model successfully predicts the most-populated structures of 17 out of the 25 cyclic peptides in the test dataset, and these structures are shown as orange stars. Pearson correlation coefficient (R), weighted error (WE), and weighted squared error (WSE) were calculated. Gray lines show where the fitted/predicted populations equal to the observed populations in MD simulations.

FIG. 18. The Ramachandran plot is divided into 10 regions for structural description. a, The total probability distribution of (ϕ, ψ) of cyclo-(GGGGG) (SEQ ID NO: 83). The plot is the same as FIG. 6a of the main text except that the grids with the lowest densities are colored white. b, Only grid points with a probability density larger than 0.00001 are shown and used for further cluster analysis. c, The grids in b are grouped into 10 clusters. The centroid of each cluster is marked by black dots. d, All the grid points in the Ramachandran plot are assigned to their closest centroid, forming 10 regions: Λ, λ, Γ, γ, B, β, Π, π, Z, and ζ.

FIG. 19. Universality of the binning map in FIG. 18d. The (ϕ, ψ) distributions for G, A, V, F, N, S, R, and D are from cyclo-(GGGGG) (SEQ ID NO: 83), cyclo-(AGGGG) (SEQ ID NO: 84), cyclo-(VGGGG) (SEQ ID NO: 85), cyclo-(FGGGG) (SEQ ID NO: 86), cyclo-(NGGGG) (SEQ ID NO: 87), cyclo-(SGGGG) (SEQ ID NO: 88), cyclo-(RGGGG) (SEQ ID NO: 89), and cyclo-(DGGGG) (SEQ ID NO: 90), respectively. The boundaries of the binning map are overlaid on each Ramachandran plot. Ramachandran plots of D-amino acids are not shown because their distribution is center-symmetric with that of the corresponding L-amino acids about origin (0°,) 0°.

FIG. 20. Comparison of performance of Scoring Function 1.0 and the StrEAMM Models on cyclo-(GNSRV) (SEQ ID NO: 51). Cyclo-(GNSRV) is a well-structured cyclic peptide predicted by Slough et al.²⁴ The three most-populated structures are shown, with a representative conformation shown in sticks and 100 randomly selected conformations shown in magenta lines. The actual populations observed in the MD simulations are given and compared to the predictions made by Scoring Function 1.0 and StrEAMM Models (1,2)/sys, (1,2)+(1,3)/sys, and (1,2)+(1,3)/random.

FIG. 21. The Ramachandran plot for cyclic hexapeptides is divided into 6 regions for structural description: Λ, λ, B, β, Π, and π.

FIG. 22. Linear StrEAMM (1,2)+(1,3)+(1,4) model for cyclic hexapeptides. The model considers interactions between the nearest-neighbor, next-nearest-neighbor, and third-nearest-neighbor residues, i.e., (1, 2), (1, 3) and (1, 4) interactions. The logarithm of the population of a structure can be expressed by the summation of the 18 weights and the weight related to the partition function. R groups of amino acids are represented by spheres. Different colors stand for different structural digits (see the binning map in FIG. 21).

FIG. 23. Performance of linear StrEAMM (1,2)+(1,3)+(1,4)/random. a, Comparison between the fitted populations and the actual populations of various structures observed in the MD simulations of the training dataset. b, Comparison between the populations predicted by StrEAMM (1,2)+(1,3)+(1,4)/random and the actual populations of various structures observed in the MD simulations of 50 random test sequences only structures with observed populations or predicted populations >1% are shown. Pearson correlation coefficient (R), weighted error

$$( W E = \frac{{\sum }_{i} {p}_{i , observed} \vert {p}_{i , observed} - {p}_{i , theory} \vert}{{\sum }_{i} {p}_{i , observed}} ,$$

where p_{i,theory} is the fitted population or the predicted population), and weighted squared error

$$( WSE = \frac{{\sum }_{i} {{p}_{i , observed} ( {p}_{i , observed} - {p}_{i , theory} )}^{2}}{{\sum }_{i} {p}_{i , observed}} )$$

were calculated. Gray lines show where the fitted/predicted populations equal the observed populations in MD simulations.

FIG. 24. Example of CNN StrEAMM incorporating (1, 2) interactions. a, the fingerprint representation for cyclic hexapeptide ARGVDE (SEQ ID NO: 52) is a concatenation of the 2048-bit fingerprint for each of the 6 amino acids. b, the list of the (1, 2) neighbors for the cyclic hexapeptide ARGVDE (SEQ ID NO: 52). c, the representation for cyclic hexapeptide ARGVDE (SEQ ID NO: 52) is reshaped into a 6×1×2048 array, and then stacked on top of the representation for cyclic hexapeptide RGVDEA (SEQ ID NO: 946), resulting in a 6×1×4096 array. This stacked representation easily allows a convolutional filter (depicted as a black-outlined rectangular prism) to encompass the features representing neighboring amino acids.

FIG. 25. The performance of the CNN StrEAMM models on the cyclic hexapeptide dataset and the cyclic pentapeptide dataset.

FIG. 26. The GNN StrEAMM model's graph convolutions are guided by (1, 2), (1, 3), and (1, 4) interactions. The GNN model considers each peptide as a graph such that each amino acid is one node, and the (1, 2), (1, 3), and (1, 4) interactions are guided by different edge types between each node. The model performs convolutions on the node representations based on these edges. In order to preserve the direction of the peptide backbone, each interaction type has forward and reverse edge types. Forward (1, 2) edges are dark blue, reverse (1, 2) edges are light blue, forward (1, 3) edges are dark green, reverse (1, 3) edges are light green, forward (1, 4) edges are dark purple, reverse (1, 4) edges are light purple.

FIG. 27. The performance of the GNN StrEAMM models on the cyclic hexapeptide dataset and the cyclic pentapeptide dataset.

FIG. 28. Genetic algorithms can efficiently generate sequences of a desired structure. a, The genetic algorithm is an iterative process that aims to evolve an initial random set of sequences such that each subsequent generation will be more “fit”. b, After only 5 generations, the genetic algorithm was able to recapitulate the top 10 sequences with high predicted populations of structure ΛΛBλβ determined by a complete search.

### DESCRIPTION OF THE INVENTION

Provided herein is a computation platform for cyclic peptides, computer-readable medium embedded with instructions executable by a processor of a computational platform, and methods for using the platform for the selection, synthesis, or assaying of cyclic peptides. The presently disclosed technology is capable of providing accurate and efficient methods that enable the rational design and fabrication of cyclic peptides.

The computational platform is capable of characterizing, predicting properties, or rationally designing cyclic peptides. The computational platform may generally include various input/output (I/O) modules, one or more processing units, a memory, and a communication network.

In some implementations, the computational platform may be any general-purpose computing system or device, such as a personal computer, workstation, cellular phone, smartphone, laptop, tablet, or the like. In this regard, the computational platform may be a system designed to integrate a variety of software, hardware, capabilities, and functionalities. Alternatively, and by way of particular configurations and programming, the computational platform may be a special-purpose system or device.

The computational platform may operate autonomously or semi-autonomously based on user input, feedback, or instructions. In some implementations, the computational platform may operate as part of, or in collaboration with, various computers, systems, devices, machines, mainframes, networks, and servers. For instance, the computational platform may communicate with one or more servers or databases, by way of a wired or wireless connection. Optionally, the computational platform may also communicate with various devices, hardware, and computers of an assembly line. For instance, the assembly line may include various fabrication, processing, or process control systems for the automated synthesis of cyclic peptides.

The I/O modules of the computational platform may include various input elements, such as a mouse, keyboard, touchpad, touchscreen, buttons, microphone, and the like, for receiving various selections and operational instructions from a user. The I/O modules may also include various drives and receptacles, such as flash-drives, USB drives, CD/DVD drives, and other computer-readable medium receptacles, for receiving various data and information. To this end, I/O modules may also include a number of communication ports and modules capable of providing communication via Ethernet, Bluetooth, or WiFi, to exchange data and information with various external computers, systems, devices, machines, mainframes, servers, networks, and the like. In addition, the I/O modules may also include various output elements, such as displays, screens, speakers, LCDs, and others.

The processing unit(s) may include any suitable hardware and components designed or capable of carrying out a variety of processing tasks, including steps implementing the present framework for quantum structure simulation. To do so, the processing unit(s) may access or receive a variety of cyclic peptide information, as will be described. The cyclic peptide information may be stored or tabulated in the memory, in the storage server(s), in the database(s), or elsewhere. In addition, such information may be provided by a user via the I/O modules, or selected based on user input.

In some configurations, the processing unit(s) may include a programmable processor or combination of programmable processors, such as central processing units (CPUs), graphics processing units (GPUs), and the like. In some implementations, the processing unit(s) may be configured to execute instructions stored in a non-transitory computer readable-media of the memory. The non-transitory computer-readable media may be included in the memory, it may be appreciated that instructions executable by the processing unit(s) may be additionally, or alternatively, stored in another data storage location having non-transitory computer-readable media.

In some embodiments, a non-transitory computer-readable medium is embedded with, or includes, instructions for receiving, using an input of the computational platform, parameter information corresponding to a cyclic peptide, and generating, using a processor or processing unit(s) of the computational platform, a cyclic peptide model based on the parameter information received. The medium may also include instructions for determining, using the processor or processing unit(s), at least one property of the quantum structure, and generating a report indicative of the at least one property determined.

In some configurations, the processing unit(s) may include one or more dedicated processing units or modules configured (e.g. hardwired, or pre-programmed) to carry out steps, in accordance with aspects of the present disclosure. Each solver module may be configured to perform a specific set of processing steps, or carry out a specific computation, and provide specific results

Solver modules of the processing unit(s) may operate independently, or in cooperation with one another. In the latter case, the modules can exchange information and data, allowing for more efficient computation, and thereby improvement in the overall processing by the processing unit(s).

As appreciated from the above, having specialized solver modules allows multiple calculations to be performed simultaneously or in substantial coordination, thereby increasing processing speed. In addition, sharing data and information between the different solver modules can prevent duplication of time-consuming processing and computations, thereby increasing overall processing efficiency.

In some implementations, the processing unit(s) may also generate various instructions, design information, or control signals for synthesizing cyclic peptides, in accordance with computations performed. For example, based on computed properties, the processing unit(s) may identify and provide an optimal method for designing or synthesizing the cyclic peptide.

The processing unit(s) may also be configured to generate a report and provide it via the I/O modules. The report may be in any form and provide various information. For instance, the report may include various numerical values, text, graphs, maps, images, illustrations, and other renderings of information and data. In particular, the report may provide various information or properties generated by the processing unit(s) for one or more cyclic peptides. The report may also include various instructions, design information, or control signals for synthesizing a cyclic peptide. To this end, the report may be provided to a user, or directed via the communication network to an assembly line or various hardware, computers or machines therein.

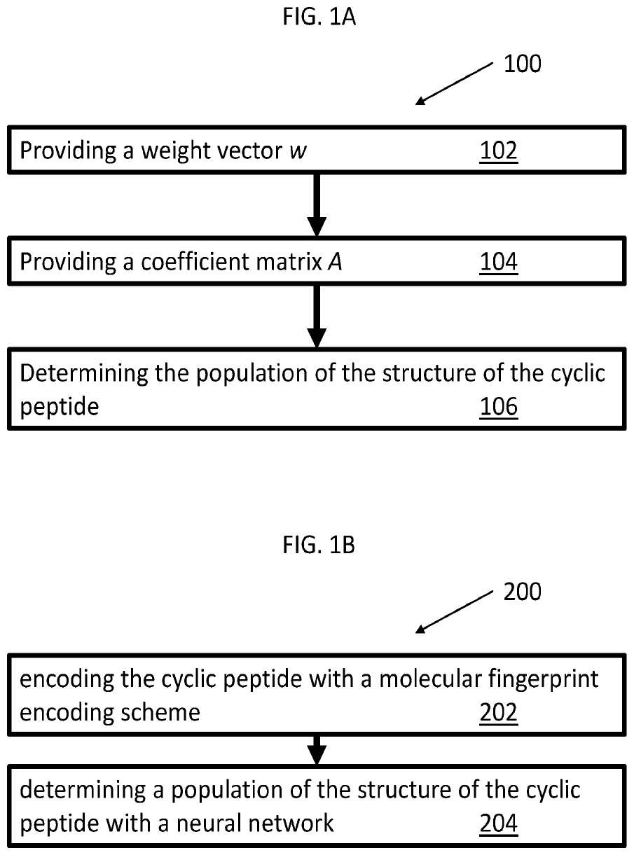

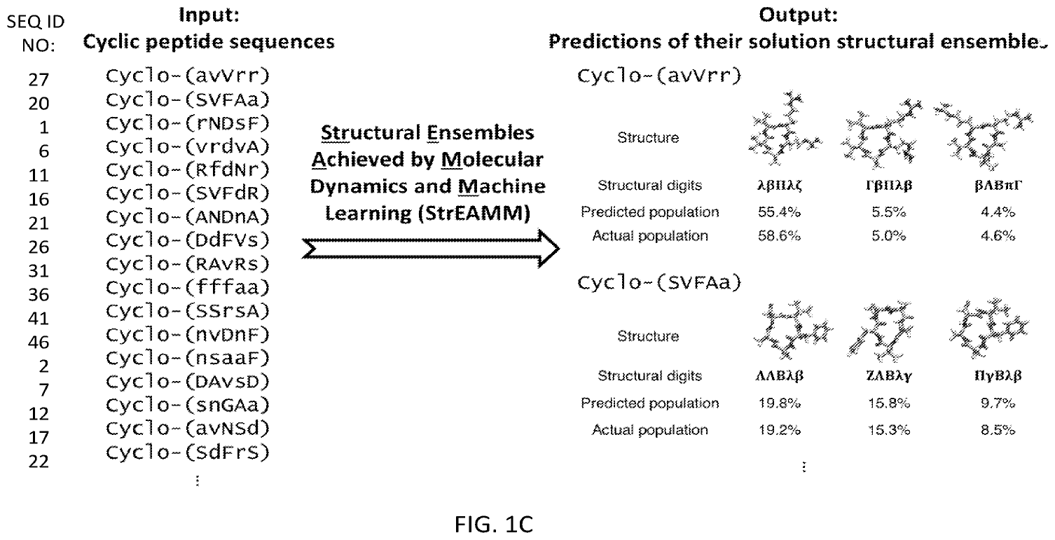

Referring now to FIGS. 1A and 1B, a flowchart setting forth steps of a process 100 and 200, respectively, in accordance with aspects of the present disclosure, is shown. Steps of process 100 or 200 may be carried out using any suitable device, apparatus, or system, such as the computational platform described herein. Steps of process 100 or 200 may be implemented as a program, firmware, software, or instructions that may be stored in non-transitory computer readable media and executed by a general-purpose, programmable computer, processor, or other suitable computing device. In some implementations, steps of process 100 or 200 may also be hardwired in an application-specific computer, processor or dedicated module.

As shown in FIG. 1A, the process 100 may begin with receiving, using an input of a computational platform, various parameter information corresponding to a cyclic peptide. Parameter information may be provided by user, and/or accessed from a memory, server, database, or other storage location. The cyclic peptide information may comprise structural and chemical information, including the number of amino acids comprising the cyclic polypeptide, the ordered arrangement of the amino acids, the connectivity of the amino acids. Based on the cyclic peptide information, a weight vector w is provided 102. The weight vector w comprises a multiplicity pairwise residue weights of an adopted structure and a multiplicity of partition function weights.

The multiplicity of pairwise residue weights of the adopted structure and the multiplicity of partition function weights are determined by minimizing the difference between a predicted population and an actual population observed in a training dataset. The dataset may be obtained from a molecular dynamics simulation. A coefficient matrix A is also provided 104. The coefficient matrix A is configured to select which of the multiplicity pairwise residue weights of the adopted structure and which one of the multiplicity of partition function weights are used to determine the population of a cyclic peptide adopting the structure. The population of the structure of the cyclic peptide can be determined from the multiplicity of pairwise residue weights and multiplicity of partition function weights 106.

In some embodiments, a neural network is used to determine the multiplicity of pairwise residue weights of the adopted structure and the multiplicity of partition function weights. As shown in FIG. 1B, the process 200 may begin with receiving, using an input of a computational platform, various parameter information corresponding to a cyclic peptide. Parameter information may be provided by user, and/or accessed from a memory, server, database, or other storage location. The cyclic peptide information may comprise structural and chemical information, including the number of amino acids comprising the cyclic polypeptide, the ordered arrangement of the amino acids, the connectivity of the amino acids. Based on the cyclic peptide information, the cyclic peptide is encoded with a molecular fingerprint encoding scheme 202. Molecular fingerprints encode structural characteristics as a vector. Molecular fingerprints can be used for fast similarity comparisons forming the basis for structure-activity relationship studies, virtual screening, construction of chemical space maps, and the like. The population of the structure of the cyclic peptide can be determined with a neural network, such as a graph neural network or a convolutional neural network 206.

The method may optionally comprise one or more additional steps. In some embodiments, one or more cyclic peptides are selected or identified based on a particular property. Cyclic peptides selected or identified by the methods disclosed herein may be synthesized according to methods known in the art for preparing cyclic peptides and/or assayed to experimentally determine their properties. For example, cyclic peptides may be selected or identified because the cyclic peptide is identified as a well-structured cyclic peptide or any other property determined by the methodology.

Using molecular dynamics simulation results as training datasets, machine-learning models may be employed that can provide molecular-dynamics-simulation-quality predictions of structural ensembles for cyclic pentapeptides in the whole sequence space. The prediction for each cyclic peptide can be made in less than 1 second of computation time. Even for the most challenging classes of poorly-structured cyclic peptides with broad conformational ensembles, the Examples demonstrate predictions were similar to those one would normally obtain from running days of explicit-solvent molecular dynamics simulations. The resulting method, termed StrEAMM (structural ensembles achieved by molecular dynamics and machine learning), efficiently predicts complete structural ensembles of cyclic peptides without relying on additional molecular dynamics simulations, constituting a seven-order-of-magnitude improvement in speed while retaining the same accuracy as explicit-solvent simulations.

Cyclic peptides are polypeptide chains which contain a circular sequence of bonds. This can be through a connection between the amino and carboxyl ends of the peptide; a connection between the amino end and a side chain; the carboxyl end and a side chain; or two side chains or more complicated arrangements. Cyclic peptides may be composed of naturally occurring or non-naturally occurring amino acid resides. The amino acid resides may be composed of L-amino acids, D-amino acids, or any combination thereof. Their length can range from just two amino acid residues to hundreds. In some embodiments, the cyclic peptide comprises 4, 5, 6, 7, 8, 9, 10, 11, or 12 amino acid residues.

Some cyclic peptides found in nature have been identified as antimicrobial or toxic. Cyclic peptides may be used for a number of different applications including as therapeutic agents, for example as antibiotics and immunosuppressive agents. Cyclic peptides are a special class of compounds in the “beyond rule-of-five” chemical space. They have unique properties for therapeutic development. Cyclic peptides are less readily degraded during digestion or by proteolysis than linear counterparts.

Most cyclic peptides reported thus far are poorly structured and adopt multiple conformations in solution. Moreover, the ability of a cyclic peptide to adopt multiple conformations can be critical to its biological properties and functions. For example, it has been noted that the chameleonic structural properties of some cyclic peptides are likely responsible for their high cell membrane permeability. Further, there can be a dynamic balance among different conformations within an ensemble, such that when one conformation is removed from solution (for example, by binding to a target), the overall conformational ensemble rebalances back towards the depleted structure. Therefore, the structures capable of binding to a target need not be highly populated in the solution ensemble, and conformations of lower populations can play an essential role in biological activity. The ability to efficiently predict and compare the structural ensembles of various cyclic peptides would significantly advances our ability to rationally design cyclic peptides.

Recent computational methods have made strides in designing well-structured cyclic peptides that preferentially populate a single conformation. As used herein, a “well-structured cyclic peptide” is a cyclic peptide where the most populated structure is predicted to be greater than 50%. However, these methods are unfortunately unable to predict the full structural ensembles of poorly-structured cyclic peptides that adopt multiple low-population conformations in solution. For example, the software improvements have enabled researchers to design highly-structured cyclic peptides, in particular, by incorporating both L- and D-prolines. Nonetheless, for the majority of cyclic peptides, which often display many solvent-exposed backbone C—O and N—H bonds and sometimes even are associated with caged water molecules, peptide-water interactions need to be described at the molecular level. The use of an explicit-solvent model is thus critical to accurately describe their energetics and structural preferences in solution. To enable efficient simulations of cyclic peptides using explicit-solvent molecular dynamics (MD) simulations, an enhanced sampling method to cyclic peptides may be used. Such a method uses bias-exchange metadynamics to target the essential transitional motions of cyclic peptides and has enables systematic studies of cyclic-peptide variants using explicit-solvent MD simulations to identify well-structured cyclic peptides. Taking advantage of the improved simulation efficiency, simulations of basis-set cyclic-peptide sequences may be used in combination with a scoring function approach that can be used to design well-structured cyclic peptides lacking proline residues, thereby expanding the available sequence space for well-structured cyclic peptide design.

The ability to discover and design well-structured cyclic peptides is valuable, and since the most-populated structure dominates in the Boltzmann-weighted averages of simulated observables, it is more straightforward to compare the most-populated structure predicted to results from solution NMR spectroscopy to verify the accuracy of the predictions. However, the ultimate capability of describing the solution structural ensembles of both well-structured and poorly-structured cyclic peptides is essential to cyclic-peptide therapeutic development. The present technology significantly expands predictive capability from the current status of only being able to discover and design well-structured cyclic peptides to efficiently predicting the full structural ensembles of both well- and non-well-structured cyclic peptides as one would obtain in MD simulations, but in just a few seconds of computation time (FIG. 1C). The Examples show that a previous scoring function can identify well-structured cyclic peptides, it is unable to predict the behaviors of non-well-structured cyclic peptides. The Examples demonstrate the use of MD simulations to generate structural ensembles of a broad set of cyclic peptides. Using these simulation results as training datasets, we are able to train models that can predict the structural ensemble, i.e., populations of various structures, for a new cyclic-peptide sequence. This new method, Structural Ensembles Achieved by Molecular Dynamics and Machine Learning (StrEAMM), enables us to rapidly predict MD-quality structural ensembles of cyclic peptides, be they well-structured or not, with very minimal computational effort.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### StrEAMM Model (1,2)/Sys: Optimizing (1, 2) Interaction Weights to Predict Populations of Cyclic Peptide Structures

In an embodiment dubbed StrEAMM Model (1,2)/sys, we considered how the interactions between the nearest neighbors, i.e. the (1, 2) interactions, impact the structural preferences of a cyclic peptide, as the first-order approximation. The population of cyclo-(X₁X₂X₃X₄X₅) adopting a certain structure S₁S₂S₃S₄S₅,

$${p}_{{S}_{1} {S}_{2} {S}_{3} {S}_{4} {S}_{5}}^{{X}_{1} {X}_{2} {X}_{3} {X}_{4} {X}_{5}} ,$$

was related to these (1, 2) interactions as:

$${p}_{{S}_{1} {S}_{2} {S}_{3} {S}_{4} {S}_{5}}^{{X}_{1} {X}_{2} {X}_{3} {X}_{4} {X}_{5}} ∝ exp ( {w}_{{S}_{1} {S}_{2}}^{{X}_{1} {X}_{2}} + {w}_{{S}_{2} {S}_{3}}^{{X}_{2} {X}_{3}} + {w}_{{S}_{3} {S}_{4}}^{{X}_{3} {X}_{4}} + {w}_{{S}_{4} {S}_{5}}^{{X}_{4} {X}_{5}} + {w}_{{S}_{5} {S}_{1}}^{{X}_{5} {X}_{1}} ) , \qquad ( 1 )$$

where

$${w}_{{S}_{i} {S}_{i + 1}}^{{X}_{i} {X}_{i + 1}}$$

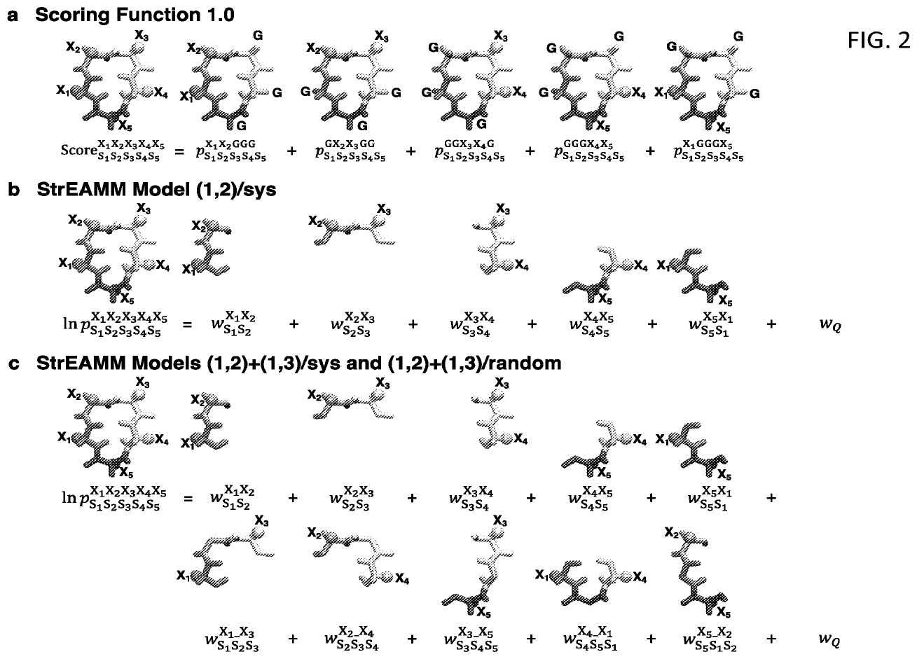

was the weight assigned to a sequential 2-residue section of the cyclic peptides when residues XᵢXᵢ₊₁ adopted structure SᵢSᵢ₊₁, Xᵢ was one of the 15 amino acids (G, A, V, F, N, S, D, R, a, v, f, n, s, d, and r; lowercase letters denote D-amino acids), and Sᵢ was one of the 10 structural digits (B, Π, Γ, Λ, Z, β, π, γ, λ, and ζ) The expression is illustrated in FIG. 2. The weights were presumed additive, sharing a similar property with energies. Since energies appear in the exponential of Boltzmann factors when related to populations, an exponential operation was also introduced here to relate the sum of the five weights to the predicted population. The operation also helped prevent the predicted populations from adopting values <0.

To obtain the exact population of cyclo-(X₁X₂X₃X₄X₅) adopting a certain structure S₁S₂S₃S₄S₅, the partition function (Q) needed to be considered:

$$Q = {\sum }_{{all}_{structures}} exp ( {w}_{{S}_{1} {S}_{2}}^{{X}_{1} {X}_{2}} + {w}_{{S}_{2} {S}_{3}}^{{X}_{2} {X}_{3}} + {w}_{{S}_{3} {S}_{4}}^{{X}_{3} {X}_{4}} + {w}_{{S}_{4} {S}_{5}}^{{X}_{4} {X}_{5}} + {w}_{{S}_{5} {S}_{1}}^{{X}_{5} {X}_{1}} ) . \qquad ( 2 )$$

$${p}_{{S}_{1} {S}_{2} {S}_{3} {S}_{4} {S}_{5}}^{{X}_{1} {X}_{2} {X}_{3} {X}_{4} {X}_{5}} = exp ( {w}_{{S}_{1} {S}_{2}}^{{X}_{1} {X}_{2}} + {w}_{{S}_{2} {S}_{3}}^{{X}_{2} {X}_{3}} + {w}_{{S}_{3} {S}_{4}}^{{X}_{3} {X}_{4}} + {w}_{{S}_{4} {S}_{5}}^{{X}_{4} {X}_{5}} + {w}_{{S}_{5} {S}_{1}}^{{X}_{5} {X}_{1}} ) / Q , \qquad ( 3 )$$

which could also be written as:

$$ln ( {p}_{{S}_{1} {S}_{2} {S}_{3} {S}_{4} {S}_{5}}^{{X}_{1} {X}_{2} {X}_{3} {X}_{4} {X}_{5}} ) = {w}_{{S}_{1} {S}_{2}}^{{X}_{1} {X}_{2}} + {w}_{{S}_{2} {S}_{3}}^{{X}_{2} {X}_{3}} + {w}_{{S}_{3} {S}_{4}}^{{X}_{3} {X}_{4}} + {w}_{{S}_{4} {S}_{5}}^{{X}_{4} {X}_{5}} + {w}_{{S}_{5} {S}_{1}}^{{X}_{5} {X}_{1}} - ln Q . \qquad ( 4 )$$

However, Eq. (2) breaks the linearity of Eq. (4), making it difficult to reach convergence when solving a set of Eq. (4)'s. Hence, another independent weight is introduce for each cyclic peptide in the training set:

$${w}_{Q} = - ln Q \\ and \qquad ( 5 )$$

$$ln ( {p}_{{S}_{1} {S}_{2} {S}_{3} {S}_{4} {S}_{5}}^{{X}_{1} {X}_{2} {X}_{3} {X}_{4} {X}_{5}} ) = {w}_{{S}_{1} {S}_{2}}^{{X}_{1} {X}_{2}} + {w}_{{S}_{2} {S}_{3}}^{{X}_{2} {X}_{3}} + {w}_{{S}_{3} {S}_{4}}^{{X}_{3} {X}_{4}} + {w}_{{S}_{4} {S}_{5}}^{{X}_{4} {X}_{5}} + {w}_{{S}_{5} {S}_{1}}^{{X}_{5} {X}_{1}} + {w}_{Q} . \qquad ( 6 )$$

Each structure of each cyclic peptide in the training set contributed an Eq. (6). Together, these equations formed a nonhomogeneous linear equation group, which could be rewritten in the matrix format:

$$ln p = A w . \qquad ( 7 )$$

The logarithms of populations were arranged into an N×1 column vector, where N was the summation of the number of structure types of each cyclic peptide in the training set. Different weights were arranged into an M×1 column vector, where M was the number of weights. The coefficient matrix A controlled which weights were used to compute the population of a specific cyclic-peptide sequence adopting a specific structure. See FIG. 7 for detailed illustration of the matrix. The weights were determined by weighted least square fitting, i.e. by minimizing the following loss function with respect to weights w:

$$L ( w ) = {\sum }_{i = 1}^{N} {p}_{i} {\vert {\sum }_{j = 1}^{M} {A}_{ij} {w}_{j} - ln {p}_{i} \vert}^{2} \qquad ( 8 )$$

To predict populations of a new cyclic peptide, Eq. (3) was used, with partition function Q calculated by Eq. (2). In theory, Eq. (2) required exhaustively counting the contributions of all possible structures. In practice, we only accounted for structures that had a population larger than 0.1% (500 frames) in at least one of the cyclic peptides in the training set (Datasets 1-3). See List 1 for the resulting structure pool that included 550 structures. Due to the incompleteness of the structure pool, we introduced a compensation factor f when computing Q. To estimate f, we computed the sum of the populations of these 550 structures for each cyclic peptide in the training set. The mean value of these summations was 0.996 and was used as the compensation factor f. The partition function used was:

$$Q = {\sum }_{{structures}_{in the pool}} exp ( {w}_{{S}_{1} {S}_{2}}^{{X}_{1} {X}_{2}} + {w}_{{S}_{2} {S}_{3}}^{{X}_{2} {X}_{3}} + {w}_{{S}_{3} {S}_{4}}^{{X}_{3} {X}_{4}} + {w}_{{S}_{4} {S}_{5}}^{{X}_{4} {X}_{5}} + {w}_{{S}_{5} {S}_{1}}^{{X}_{5} {X}_{1}} ) / f . \qquad ( 9 )$$

The predicted population was then calculated using Eq. (3) with partition function calculated using Eq. (9).

When calculating the populations using Eq. (3) for a new cyclic peptide, it was possible to encounter some weights that did not exist in the training set. The absence of these weights in the training set suggested the amino acid sequences had little tendency to adopt the corresponding structures, and these weights were thus assigned to a very negative number (−20 was used, which was small enough to bring the final predicted population to essentially zero).

The dataset used in the training for StrEAMM Model (1,2)/sys was dubbed Dataset 1. The matrix equation (7) contained 131,779 linear equations and 6,101 independent weights; weights that were mirror images of each other were treated as one independent weight because

$${w}_{{S}_{i} {S}_{i + 1}}^{{X}_{i} {X}_{i + 1}} = {w}_{{s}_{i} {s}_{i + 1}}^{{x}_{i} {x}_{i + 1}}$$

with capital and lowercase letter pairs representing enantiomers of amino acids and structures. The distribution of the weights is shown in FIG. 9.

### StrEAMM Models (1,2)+(1,3)/Sys and (1,2)+(1,3)/Random: Including Both (1,2) and (1,3) Interaction Weights

In embodiments dubbed StrEAMM Models (1,2)+(1,3)/sys and (1,2)+(1,3)/random, we considered interactions between the nearest neighbors and between next-nearest neighbors, i.e. both (1, 2) interactions and (1, 3) interactions. The population of cyclo-(X₁X₂X₃X₄X₅) adopting a certain structure S₁S₂S₃S₄S₅,

$${p}_{{S}_{1} {S}_{2} {S}_{3} {S}_{4} {S}_{5}}^{{X}_{1} {X}_{2} {X}_{3} {X}_{4} {X}_{5}} ,$$

was related to the (1, 2) and (1, 3) interactions as:

$${p}_{{S}_{1} {S}_{2} {S}_{3} {S}_{4} {S}_{5}}^{{X}_{1} {X}_{2} {X}_{3} {X}_{4} {X}_{5}} ∝ exp ( {w}_{{S}_{1} {S}_{2}}^{{X}_{1} {X}_{2}} + {w}_{{S}_{2} {S}_{3}}^{{X}_{2} {X}_{3}} + {w}_{{S}_{3} {S}_{4}}^{{X}_{3} {X}_{4}} + {w}_{{S}_{4} {S}_{5}}^{{X}_{4} {X}_{5}} + {w}_{{S}_{5} {S}_{1}}^{{X}_{5} {X}_{1}} + {w}_{{S}_{1} {S}_{2} {S}_{3}}^{{X}_{1 -} {X}_{3}} + {w}_{{S}_{2} {S}_{3} {S}_{4}}^{{X}_{2} {\_ X}_{4}} + {w}_{{S}_{3} {S}_{4} {S}_{5}}^{{X}_{3} {\_ X}_{5}} + {w}_{{S}_{4} {S}_{5} {S}_{1}}^{{X}_{4 -} {X}_{1}} + {w}_{{S}_{5} {S}_{1} {S}_{2}}^{{X}_{5} {\_ X}_{2}} ) , \qquad ( 10 )$$

where

$${w}_{{S}_{i} {S}_{i + 1} {S}_{i + 2}}^{{X}_{i} {\_ X}_{i + 2}}$$

was the weight assigned to the interactions between Xᵢ and Xᵢ₊₂ when residues XᵢXᵢ₊₁Xᵢ₊₂ adopted the structure SᵢSᵢ₊₁Sᵢ₊₂. Note that while describing (1, 3) interactions, we also included the structure of the middle residue, considering that the ø and y dihedrals of residue i+1 would affect the relative distance and orientation of residues Xᵢ and Xᵢ₊₂. However, the middle residue Xᵢ₊₁ can be any amino acid. The expression is illustrated in FIG. 2. Similar to what was done in StrEAMM Model (1,2)/sys, exact populations could be obtained by introducing the partition function Q:

$${p}_{{S}_{1} {S}_{2} {S}_{3} {S}_{4} {S}_{5}}^{{X}_{1} {X}_{2} {X}_{3} {X}_{4} {X}_{5}} = exp ( {w}_{{S}_{1} {S}_{2}}^{{X}_{1} {X}_{2}} + {w}_{{S}_{2} {S}_{3}}^{{X}_{2} {X}_{3}} + {w}_{{S}_{3} {S}_{4}}^{{X}_{3} {X}_{4}} + {w}_{{S}_{4} {S}_{5}}^{{X}_{4} {X}_{5}} + {w}_{{S}_{5} {S}_{1}}^{{X}_{5} {X}_{1}} + {w}_{{S}_{1} {S}_{2} {S}_{3}}^{{X}_{1 -} {X}_{3}} + {w}_{{S}_{2} {S}_{3} {S}_{4}}^{{X}_{2} {\_ X}_{4}} + {w}_{{S}_{3} {S}_{4} {S}_{5}}^{{X}_{3} {\_ X}_{5}} + {w}_{{S}_{4} {S}_{5} {S}_{1}}^{{X}_{4 -} {X}_{1}} + {w}_{{S}_{5} {S}_{1} {S}_{2}}^{{X}_{5} {\_ X}_{2}} ) / Q , \\ and , \qquad ( 11 )$$

$$Q = {\sum }_{{structures}_{in the pool}} exp ( {w}_{{S}_{1} {S}_{2}}^{{X}_{1} {X}_{2}} + {w}_{{S}_{2} {S}_{3}}^{{X}_{2} {X}_{3}} + {w}_{{S}_{3} {S}_{4}}^{{X}_{3} {X}_{4}} + {w}_{{S}_{4} {S}_{5}}^{{X}_{4} {X}_{5}} + {w}_{{S}_{5} {S}_{1}}^{{X}_{5} {X}_{1}} + {w}_{{S}_{1} {S}_{2} {S}_{3}}^{{X}_{1} {\_ X}_{3}} + {w}_{{S}_{2} {S}_{3} {S}_{4}}^{{X}_{2} {\_ X}_{4}} + {w}_{{S}_{3} {S}_{4} {S}_{5}}^{{X}_{3} {\_ X}_{5}} + {w}_{{S}_{4} {S}_{5} {S}_{1}}^{{X}_{4} {\_ X}_{1}} + {w}_{{S}_{5} {S}_{1} {S}_{2}}^{{X}_{5} {\_ X}_{2}} ) / f \qquad ( 12 )$$

with f being the compensation factor to account for the incompleteness of the structure pool. Again, we applied Eq. (5) when fitting for the weights with the following linear equation:

$$ln ( {p}_{{S}_{1} {S}_{2} {S}_{3} {S}_{4} {S}_{5}}^{{X}_{1} {X}_{2} {X}_{3} {X}_{4} {X}_{5}} ) = {w}_{{S}_{1} {S}_{2}}^{{X}_{1} {X}_{2}} + {w}_{{S}_{2} {S}_{3}}^{{X}_{2} {X}_{3}} + {w}_{{S}_{3} {S}_{4}}^{{X}_{3} {X}_{4}} + {w}_{{S}_{4} {S}_{5}}^{{X}_{4} {X}_{5}} + {w}_{{S}_{5} {S}_{1}}^{{X}_{5} {X}_{1}} + {w}_{{S}_{1} {S}_{2} {S}_{3}}^{{X}_{1 -} {X}_{3}} + {w}_{{S}_{2} {S}_{3} {S}_{4}}^{{X}_{2 -} {X}_{4}} + {w}_{{S}_{3} {S}_{4} {S}_{5}}^{{X}_{3 -} {X}_{5}} + {w}_{{S}_{4} {S}_{5} {S}_{1}}^{{X}_{4 -} {X}_{1}} + {w}_{{S}_{5} {S}_{1} {S}_{2}}^{{X}_{5 -} {X}_{2}} + {w}_{Q} . \qquad ( 13 )$$

Each structure of each cyclic peptide in the training set contributed an Eq. (13). Together, these equations formed a matrix equation (7). The optimized weights were obtained by minimizing the loss function (8). The predicted population of a new cyclic peptide adopting a specific structure was calculated by Eq. (11) with Q calculated via Eq. (12).

We used the SciPy package of Python language to build the matrix and calculate the weights. The loss function in Eq. 8 was minimized by the scipy.sparse.linalg.lsqr function of the package.

StrEAMM Model (1,2)+(1,3)/Sys: Training with Dataset 2

The matrix equation (7) contained 251,120 linear equations and 34,100 independent weights, including 6,123 (1, 2) interaction weights and 27,977 (1, 3) interaction weights. The distributions of the weights are shown in FIG. 11.

StrEAMM Model (1,2)+(1,3)/Random: Training with Dataset 3

The matrix equation (7) contained 465,728 linear equations and 44,439 independent weights, including 7,626 (1,2) interaction weights and 36,813 (1, 3) interaction weights. The distributions of weights related to (1, 2) interactions and (1, 3) interactions are shown in FIG. 13. To avoid large errors in the weight estimates, if a weight occurred fewer than 10 times in the training set, it was assigned a very negative number (−20 was used, which was small enough to bring the final predicted population to essentially zero) when calculating a population.

StrEAMM Model (1,2)+(1,3)/sys37: Training with Dataset 5

Dataset 5 was an extension of Dataset 2 by including the basic amino acids in L or D configurations except Pro (37 amino acids total). The reason we exclude Pro is that it increases the likelihood of observing a cis peptide bond and we believe the current force fields are not trained to and are unable to predict cis/trans configurations correctly. The new training dataset (Dataset 5) included 1,315 systematic sequences: Cyclo-(GGGGG) (SEQ ID NO: 83), cyclo-(X₁ GGGG), cyclo-(X₁X₂GGG), cyclo-(X₁x₂GGG), cyclo-(X₁ GX₂GG), and cyclo-(X₁ Gx₂GG), with Xᵢ being one of the 18 L-amino acids and Xᵢ being one of the 18 D-amino acids. Each sequence contained one unique nearest-neighbor or next-nearest-neighbor pair with the rest of the sequence filled by Gly's. Again, the enantiomers of these cyclic peptides were not simulated, and their structural ensembles were inferred from the 1,314 simulated cyclic peptides.

A new test dataset including the 37 types of amino acids was built (Dataset 6, List 4). The performance of the model is shown in FIG. 15. StrEAMM Model (1,2)+(1,3)/sys37 successfully predicted the most-populated structures of 51 of the 75 test cyclic peptides (stars in FIG. 19). The Pearson correlation coefficient was 0.841 when comparing the predicted and the observed populations, and the weighted error was 4.097. Comparing to StrEAMM model (1,2)+(1,3)/sys, which successfully predicted the most-populated structures of 30 of the 50 test cyclic peptides and whose Pearson correlation coefficient was 0.912, the performance of StrEAMM model (1,2)+(1,3)/sys37 only had minor deterioration in Pearson correlation coefficient, but successfully predicted the most-populated structures of more cyclic peptides. The comparable performance of StrEAMM model (1,2)+(1,3)/sys and (1,2)+(1,3)/sys37 indicates the extendable of StrEAMM model to other types of amino acids.

To build a training dataset with a similar strategy as Dataset 3 while including 37 types of L- and D-amino acids, one should simulate >10,131 (37×37×37/5) cyclic pentapeptides, which is unfeasible due to the limited computational resources as present except on supercomputers. The performance of the StrEAMM model will have significant improvement with a larger training dataset, however.

### Extant Scoring Function Cannot Predict the Structural Ensembles of Non-Well-Structured Cyclic Peptides.

We began by building and testing a scoring function analogous to the one developed by Slough et al.²⁴ but with two major improvements. First, Slough et al. described a cyclic-pentapeptide structure using specific turn combinations (some type of β turn at residues i and i+1 and some type of tight turn at residue i+3). Because cyclic pentapeptides can adopt conformations other than these canonical turn combinations, we separated the (ϕ, ψ) space into 10 different regions and denoted each region with a structural digit (B, Π, Γ, Λ, Z, β, π, γ, λ, or ζ). Thus, a cyclic-pentapeptide structure can be described using a 5-letter code (for example, λβΠλζ). Second, while Slough et al. used a dataset containing 57 cyclo-(X₁X₂AAA) peptides with Xᵢ being one of the eight amino acids (G, A, V, F, N, S, D, and R), we used 106 cyclo-(X₁X₂GGG) peptides with Xᵢ being one of the 15 amino acids: G, A, V, F, N, S, D, R, a, v, f, n, s, d, and r. In the dataset, each sequence contained one unique nearest-neighbor pair with the rest of the sequence filled by Gly's (Dataset 1). The new dataset was also extended to include D-amino acids, which are commonly used in cyclic-peptide drug development efforts both to improve the capability of stabilizing desired conformations and to reduce enzymatic degradation. In this scoring function, herein termed Scoring Function 1.0, the score of cyclo-(X₁X₂X₃X₄X₅) adopting a specific structure S₁S₂S₃S₄S₅ was computed as:

$${Score}_{{S}_{1} {S}_{2} {S}_{3} {S}_{4} {S}_{5}}^{{X}_{1} {X}_{2} {X}_{3} {X}_{4} {X}_{5}} = {p}_{{S}_{1} {S}_{2} {S}_{3} {S}_{4} {S}_{5}}^{{X}_{1} {X}_{2} GGG} + {p}_{{S}_{1} {S}_{2} {S}_{3} {S}_{4} {S}_{5}}^{{GX}_{2} {X}_{3} GG} + {p}_{{S}_{1} {S}_{2} {S}_{3} {S}_{4} {S}_{5}}^{{GGX}_{3} {X}_{4} G} + {p}_{{S}_{1} {S}_{2} {S}_{3} {S}_{4} {S}_{5}}^{{GGGX}_{4} {X}_{5}} + {p}_{{S}_{1} {S}_{2} {S}_{3} {S}_{4} {S}_{5}}^{{X}_{1} {GGGX}_{5}} , \qquad ( 14 )$$

where

$${p}_{{S}_{1} {S}_{2} {S}_{3} {S}_{4} {S}_{5}}^{{X}_{1} {X}_{2} GGG}$$

was the population of structure S₁S₂S₃S₄S₅ observed in the cyclo-(X₁X₂GGG) simulation, and so forth (FIG. 2 a). Ideally, the five parent sequences, X₁X₂GGG, GX₂X₃GG, GGX₃X₄G, GGGX₄X₅, X₁GGGX₅ would capture how nearest-neighbor pairs X₁X₂, X₂X₃, X₃X₄, X₄X₅, and X₅X₁ impact the structural preferences of cyclo-(X₁X₂X₃X₄X₅).

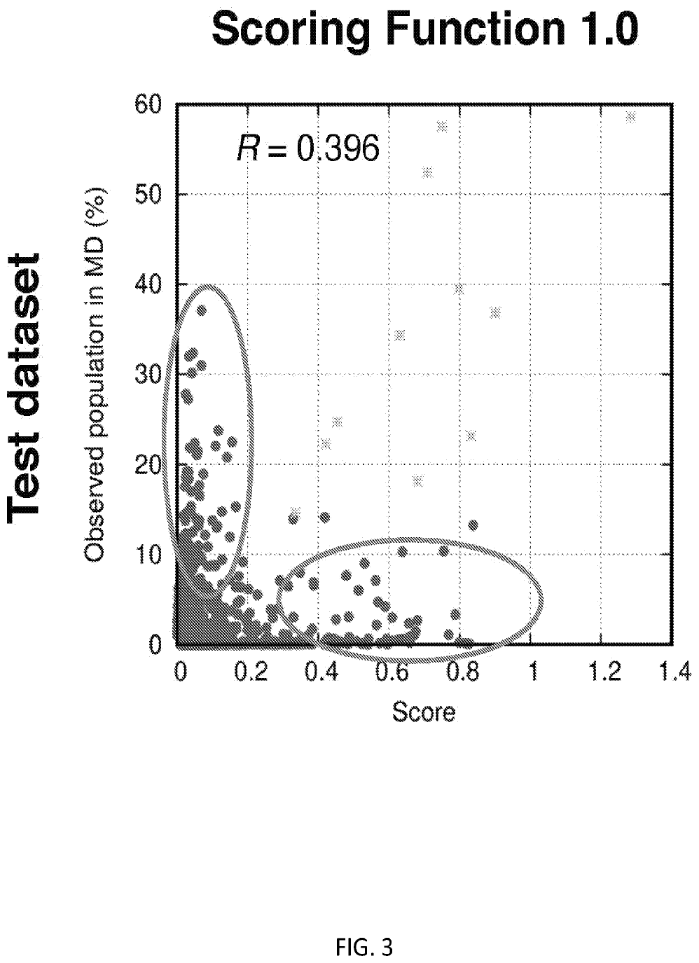

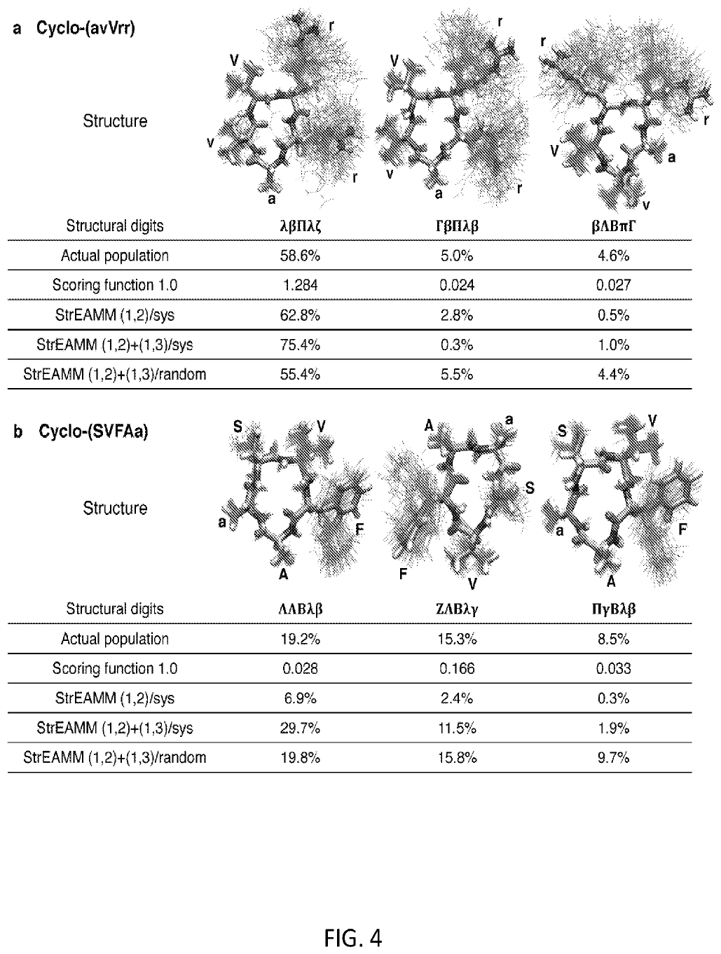

To evaluate the performance of the scoring functions, we ran MD simulations of 50 cyclic peptides with random sequences and used their structural ensembles as the test dataset (see List 2 for the exact sequences). FIG. 3 shows the performance of Scoring Function 1.0 for predicting the populations of specific structures adopted by these 50 random sequences. We found the scoring function successfully predicted the most-populated structures of 11 out of the 50 test cyclic peptides (stars in FIG. 3; also see FIG. 8, boxed). Three cyclic peptides in the test dataset were considered well-structured, i.e. the population of the most-populated structure was >50%, and their most-populated structures were all predicted successfully. These data suggested that Scoring Function 1.0 was capable of identifying well-structured sequences. However, for structures with low populations, the scores and the observed populations in MD simulations showed a poor correlation (highlighted by circles in FIG. 3; the Pearson correlation coefficient of all the data points was 0.312), suggesting that Scoring Function 1.0 was unable to predict the behaviors of non-well-structured cyclic peptides. To further highlight this issue, in FIG. 4 we showed the structures and populations of the three most-populated conformations observed in the simulations of a well-structured cyclic peptide, cyclo-(avVrr), and in a non-well-structured cyclic peptide, cyclo-(SVFAa), along with the scores predicted by Scoring Function 1.0. While Scoring Function 1.0 provided scores that correlated well with the populations of the three most-populated conformations for the well-structured cyclo-(avVrr) (scores of 1.284, 0.024, and 0.027 vs. the actual populations of 58.6%, 5.0%, and 4.6% observed in the MD simulations, respectively), it was unable to predict the behavior of the non-well-structured cyclo-(SVFAa) (scores of 0.028, 0.166, and 0.033 vs. the actual populations of 19.2%, 15.3%, and 8.5% observed in the MD simulations, respectively).

### StrEAMM Model (1,2)/Sys: Optimizing (1, 2) Interaction Weights to Predict Populations of Cyclic Peptide Structures

We found that Scoring Function 1.0 was unable to predict populations of structures that were not highly populated (FIG. 3) and could not be used to describe conformational ensembles of non-well-structured cyclic peptides. In Scoring Function 1.0, the predicted score was a simple summation of the populations observed in the MD simulations of the five parent sequences—the higher the score, the more likely that a structure was preferred. Examination of Eq. (14) suggests that if a structure does not populate highly in the training dataset, i.e., in cyclo-(X₁X₂GGG) peptides, then there is little chance for cyclic peptides of any sequences to be predicted to have a large population for that particular structure. We hypothesized that the issue results from the requirement of simply summing the five populations to obtain the score and that these populations are strictly derived from cyclo-(X₁X₂GGG). Thus, a different scoring scheme that is not merely summing the populations observed in the MD simulations of the five parent sequences, but somehow extracts and embeds effective (1, 2) interaction contributions on a cyclic peptide's structural preferences is needed. Furthermore, in Scoring Function 1.0, the populations observed in the MD simulations of the parent sequences were summed to obtain a score; however, the exact relationship between a score and the population was unclear.

Here, we devised our Structural Ensembles Achieved by Molecular Dynamics and Machine Learning (StrEAMM) Model (1,2)/sys to estimate the populations more directly from the training dataset. In StrEAMM Model (1,2)/sys, the predicted population of cyclo-(X₁X₂X₃X₄X₅) adopting a specific structure S₁S₂S₃S₄S₅ was computed as:

$${p}_{{S}_{1} {S}_{2} {S}_{3} {S}_{4} {S}_{5}}^{{X}_{1} {X}_{2} {X}_{3} {X}_{4} {X}_{5}} = exp ( {w}_{{S}_{1} {S}_{2}}^{{X}_{1} {X}_{2}} + {w}_{{S}_{2} {S}_{3}}^{{X}_{2} {X}_{3}} + {w}_{{S}_{3} {S}_{4}}^{{X}_{3} {X}_{4}} + {w}_{{S}_{4} {S}_{5}}^{{X}_{4} {X}_{5}} + {w}_{{S}_{5} {S}_{1}}^{{X}_{5} {X}_{1}} ) / Q . \qquad ( 15 )$$

Here,

$${w}_{{S}_{i} {S}_{i + 1}}^{{X}_{i} {X}_{i + 1}}$$

was the weight assigned when residues XᵢXᵢ₊₁ adopted structure SᵢSᵢ₊₁, Xᵢ was one of the 15 amino acids (G, A, V, F, N, S, D, R, a, v, f, n, s, d, and r), and Sᵢ was one of the 10 structural digits (B, Π, Γ, Λ, Z, β, π, γ, λ, and ζ). The expression (in the logarithmic form) is illustrated in FIG. 2 b. The weights were designed to represent the effective free energy contribution from residues XᵢXᵢ₊₁ adopting structure SᵢSᵢ₊₁ and the contributions from different nearest-neighbor pairs were presumed additive. A partition function Q and an exponential operation were introduced to convert the final effective free energy to a predicted population. The weights and the partition functions were then determined by weighted least square fitting to minimize the difference between the predicted populations and the actual populations observed in the MD simulations of the training sequences.

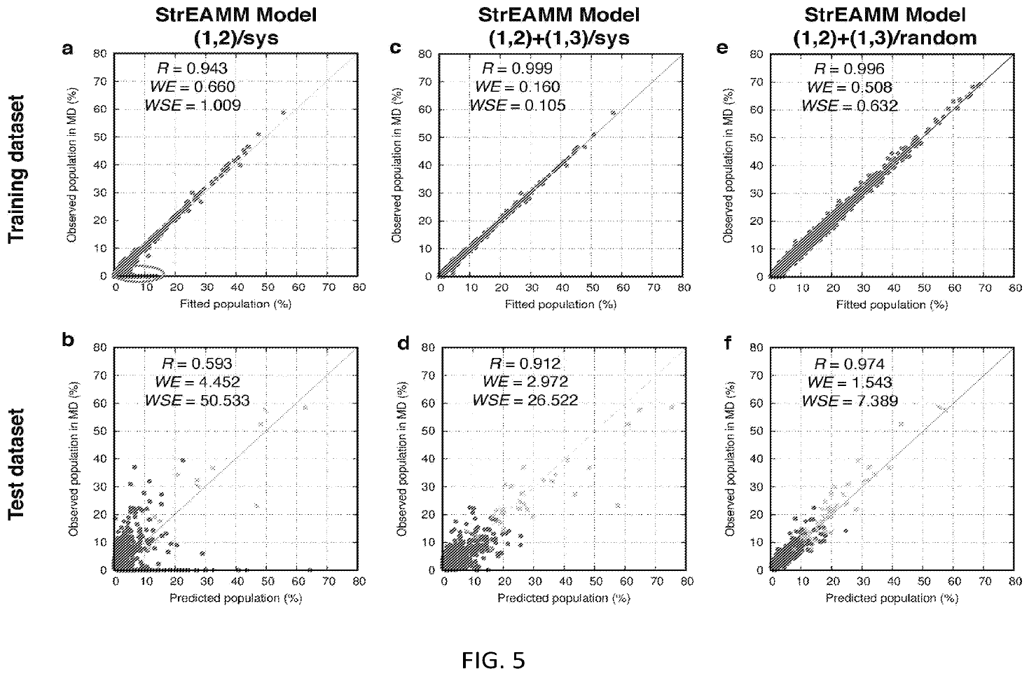

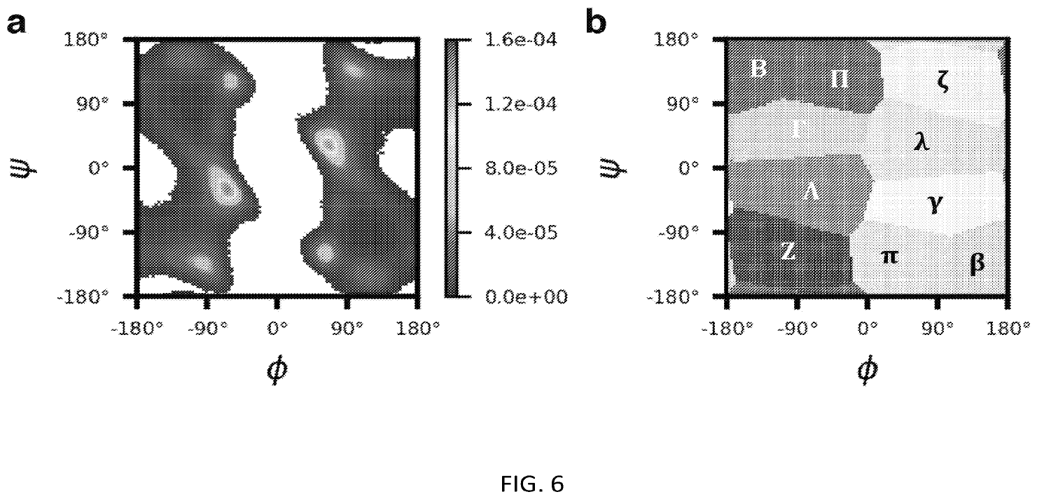

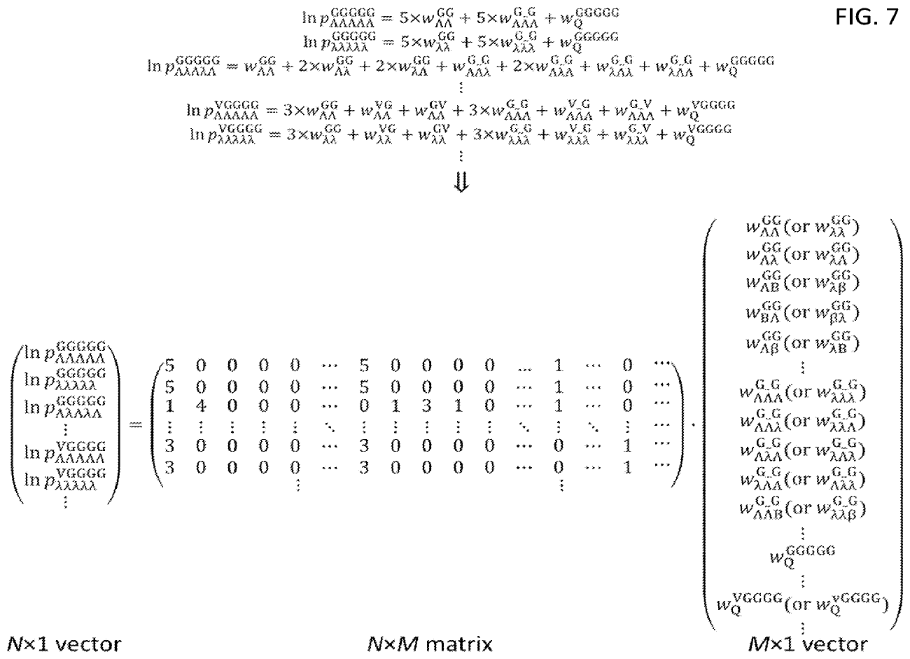

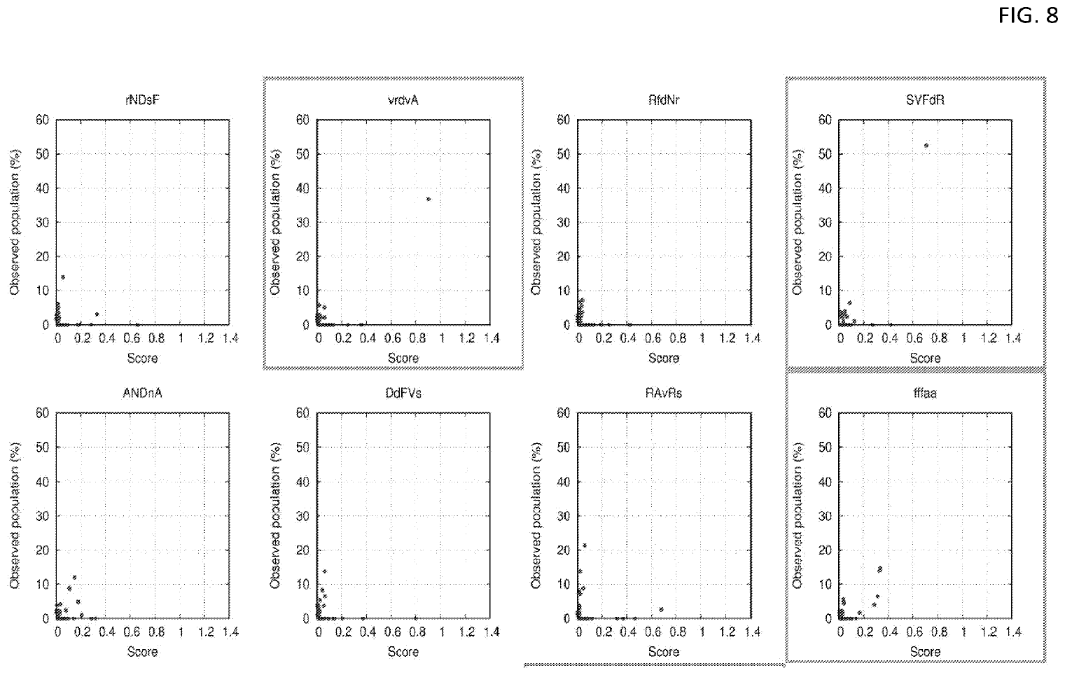

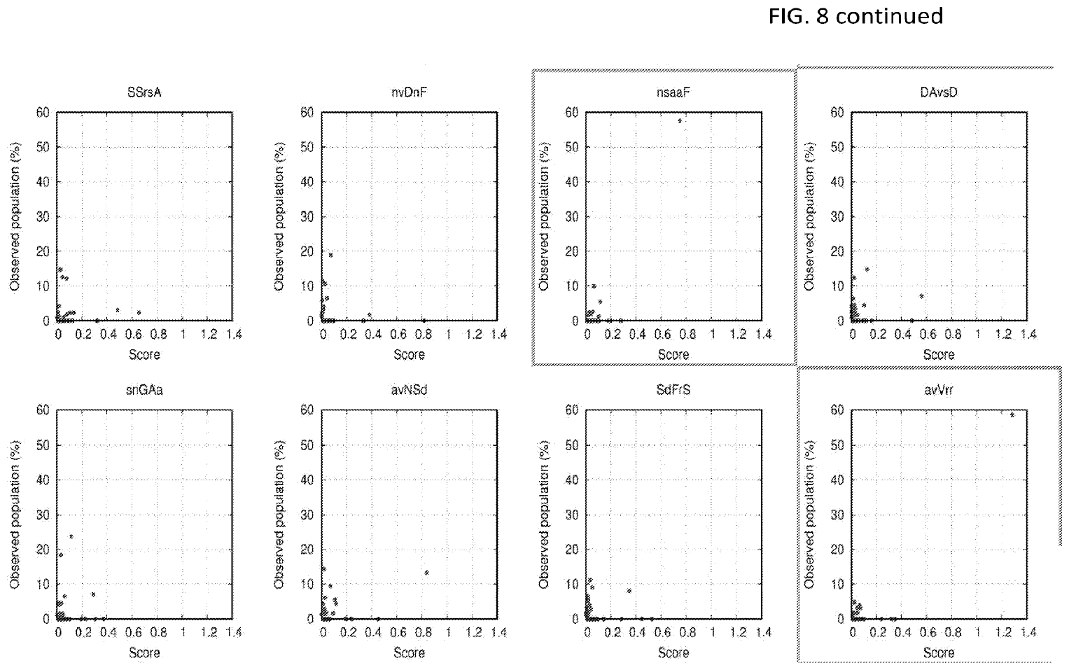

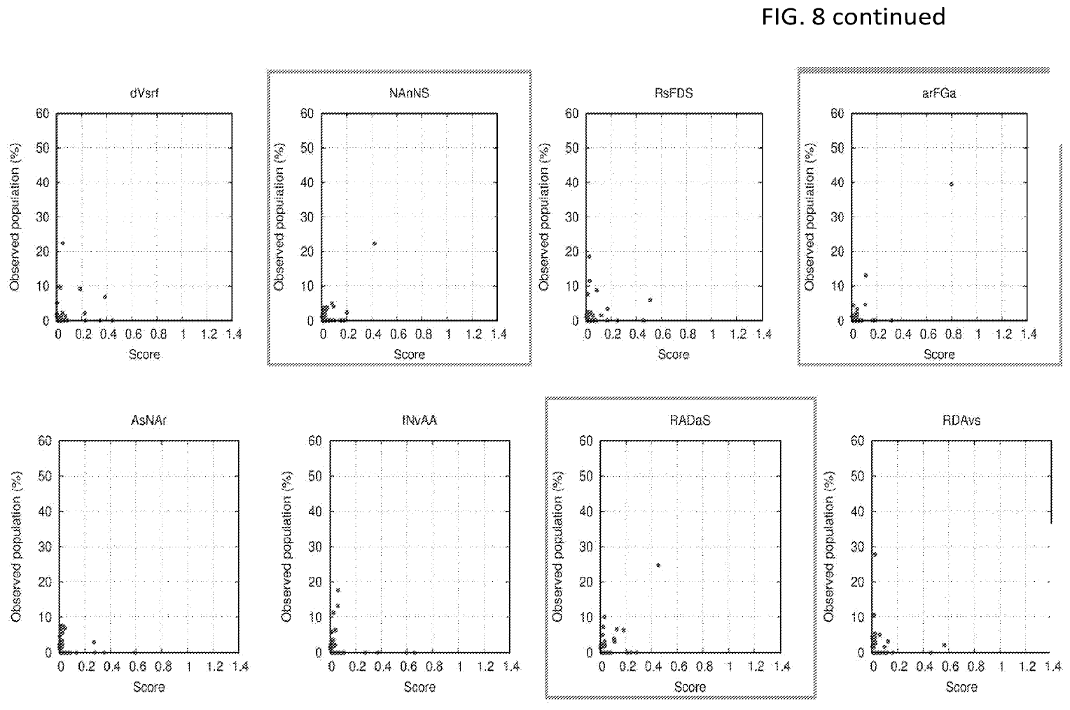

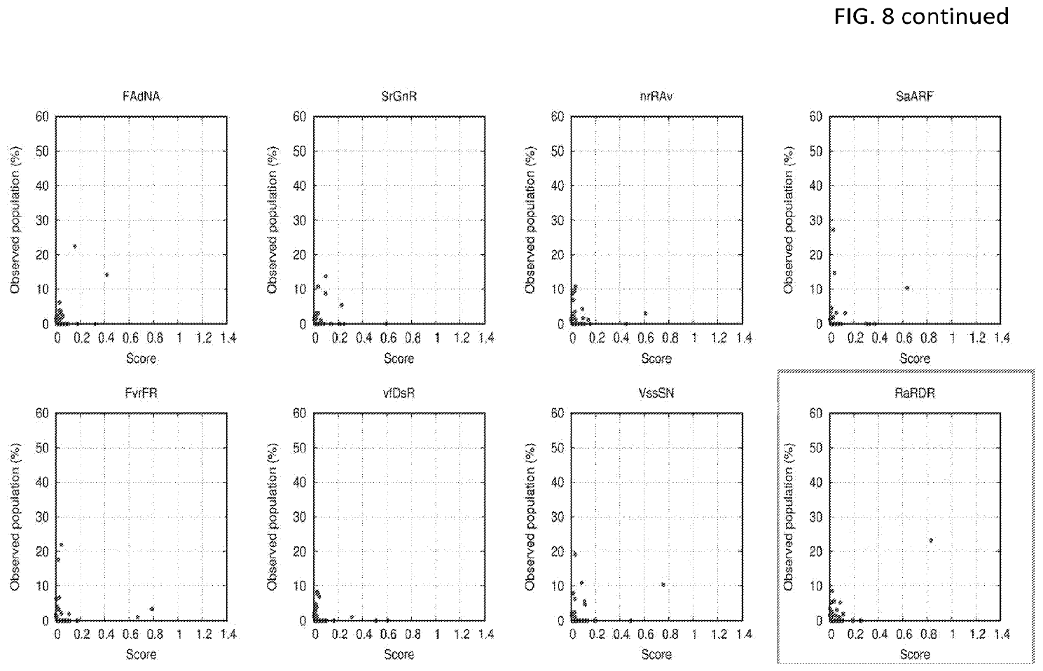

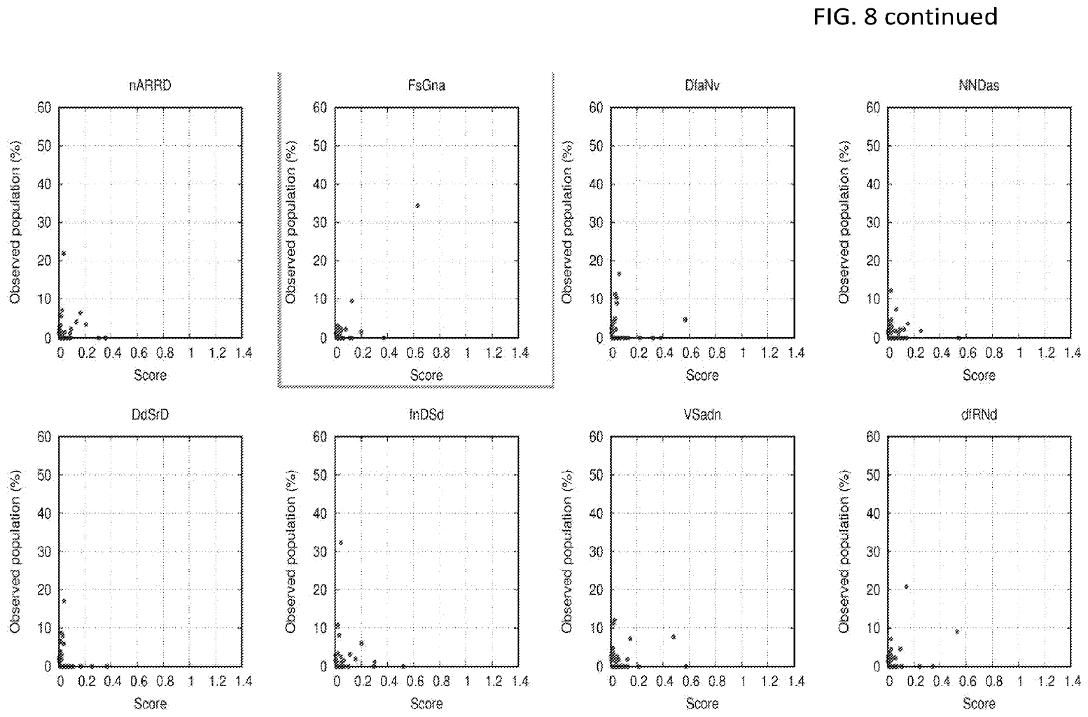

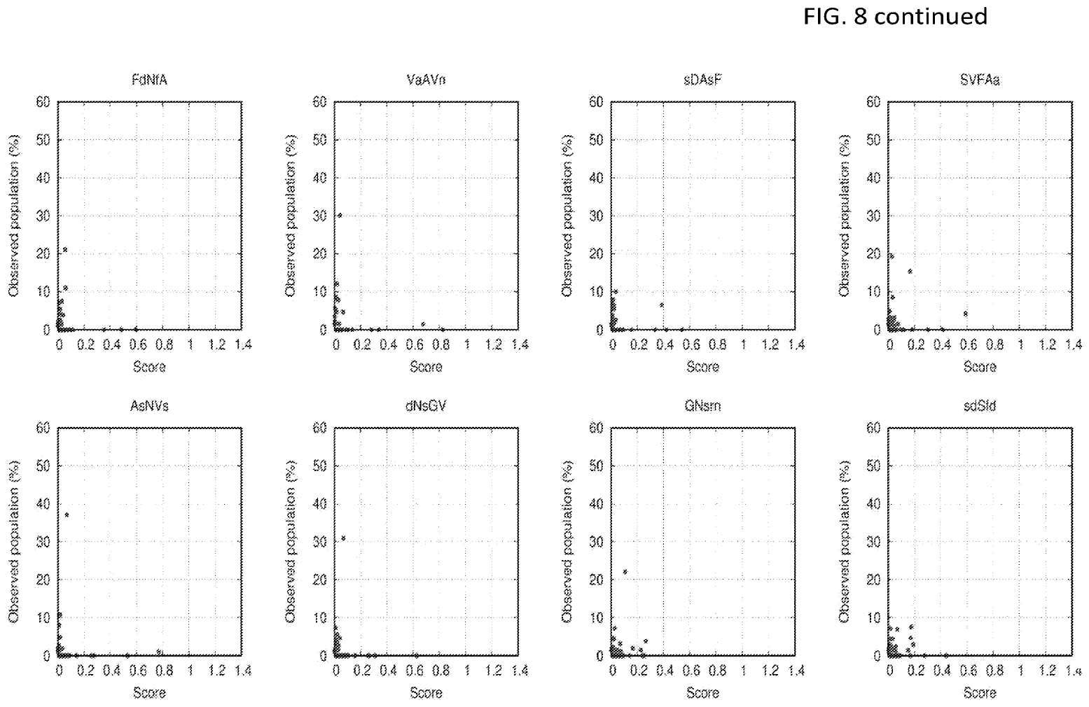

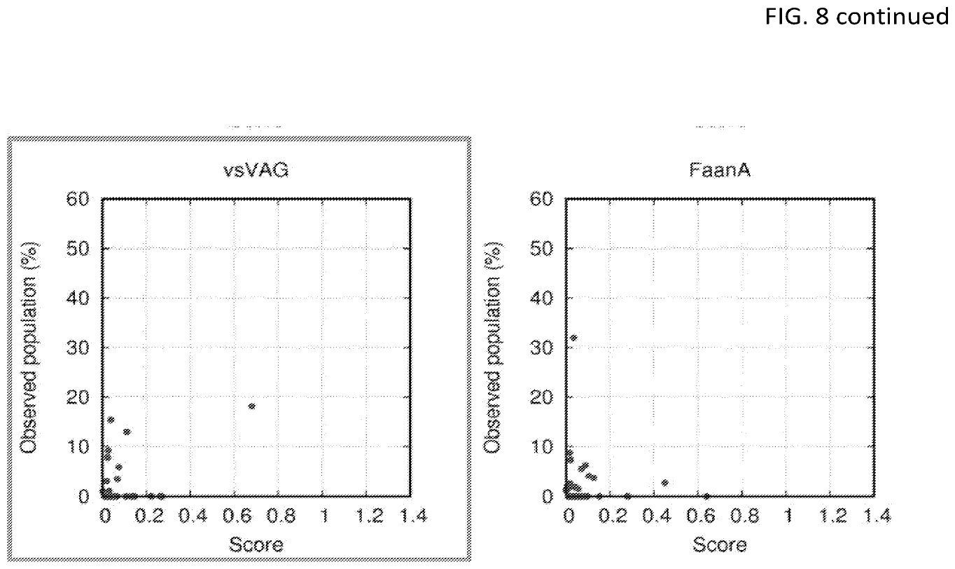

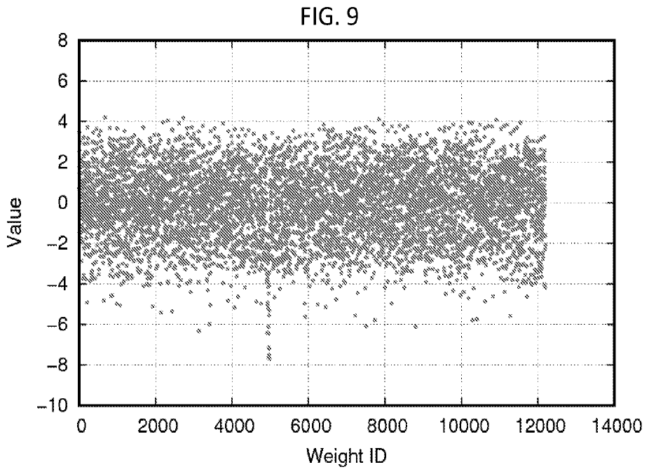

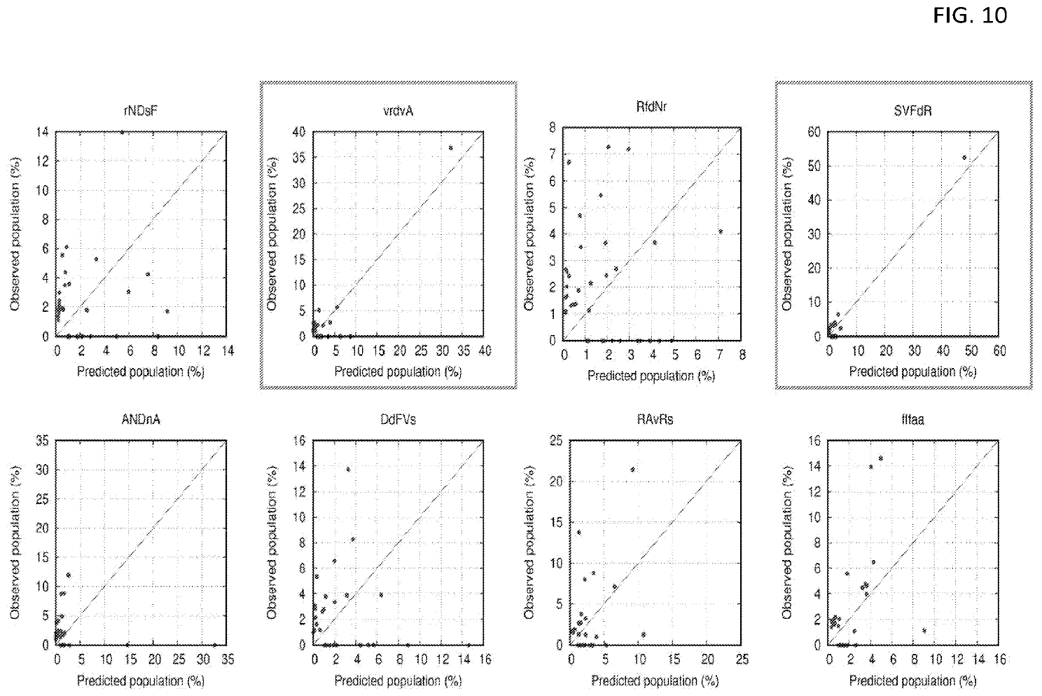

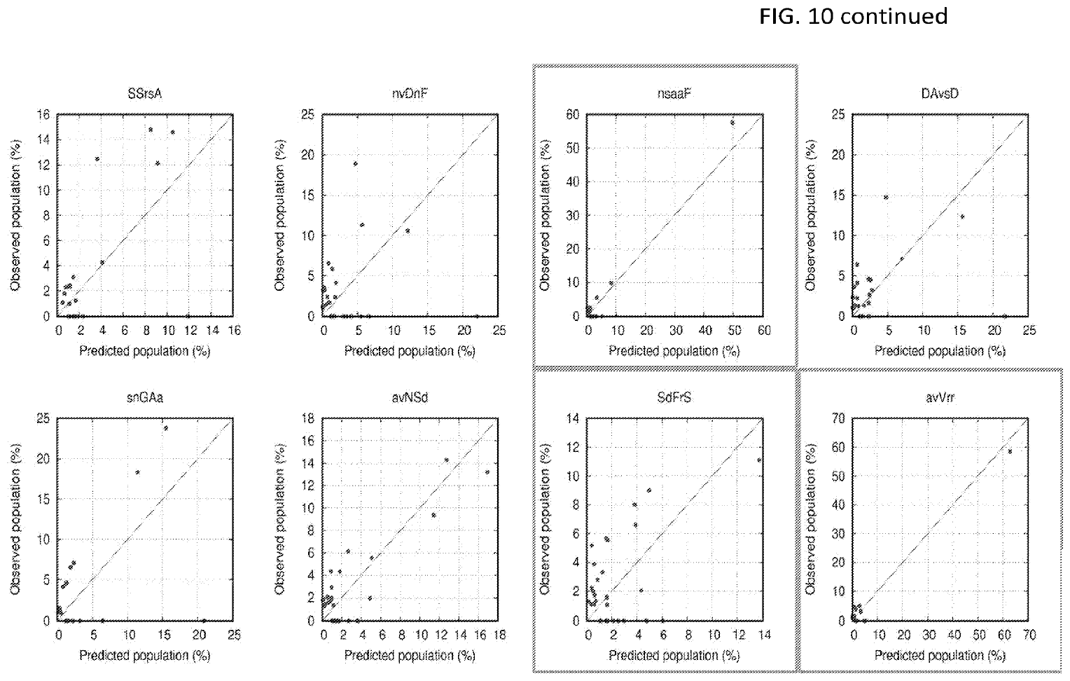

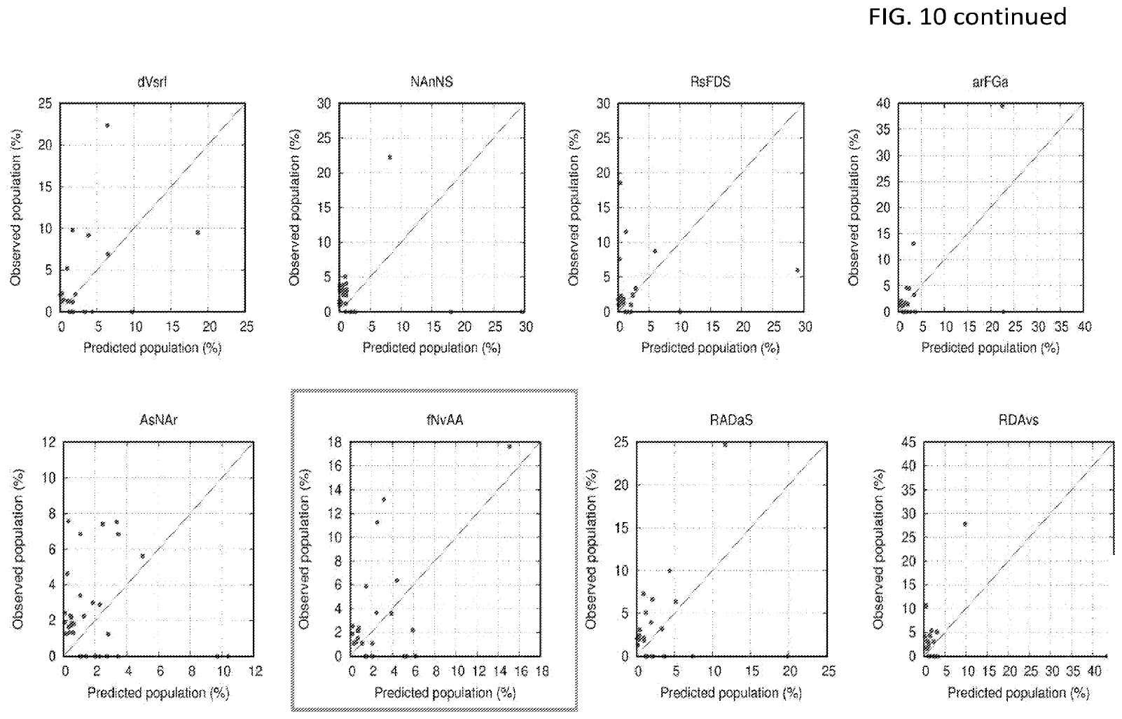

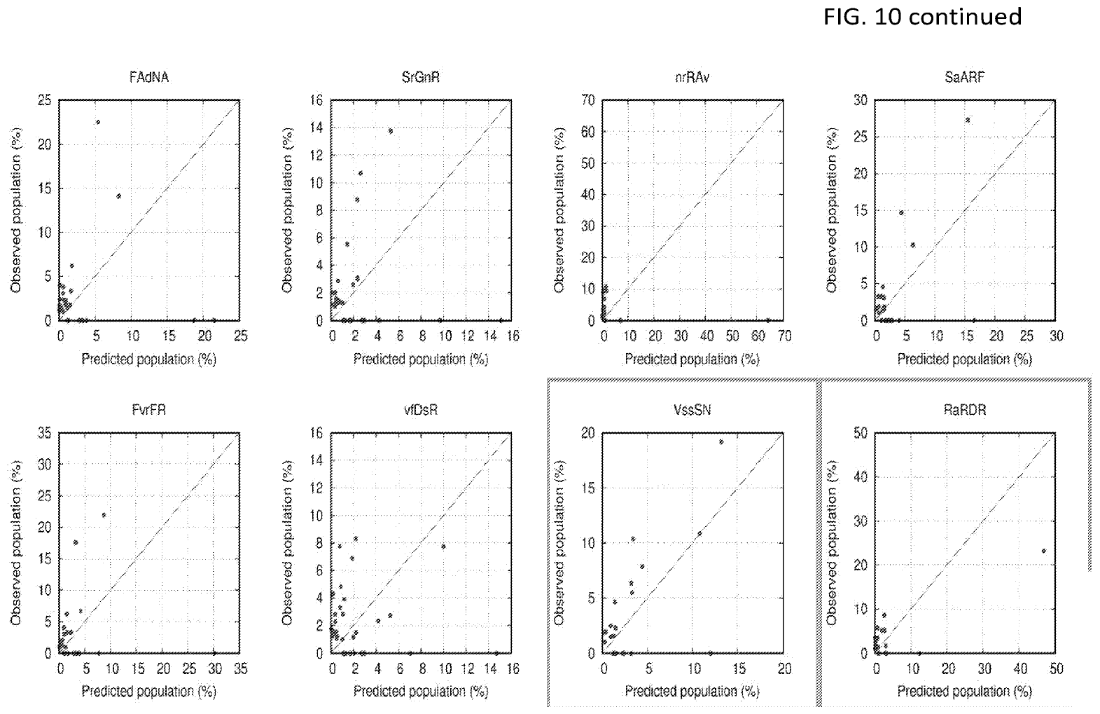

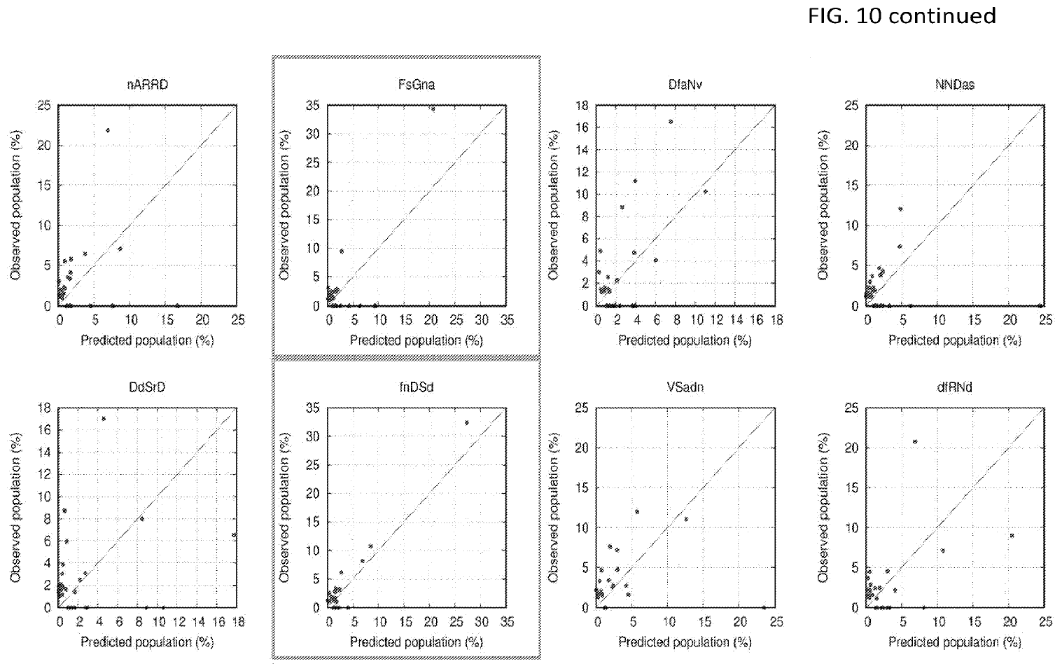

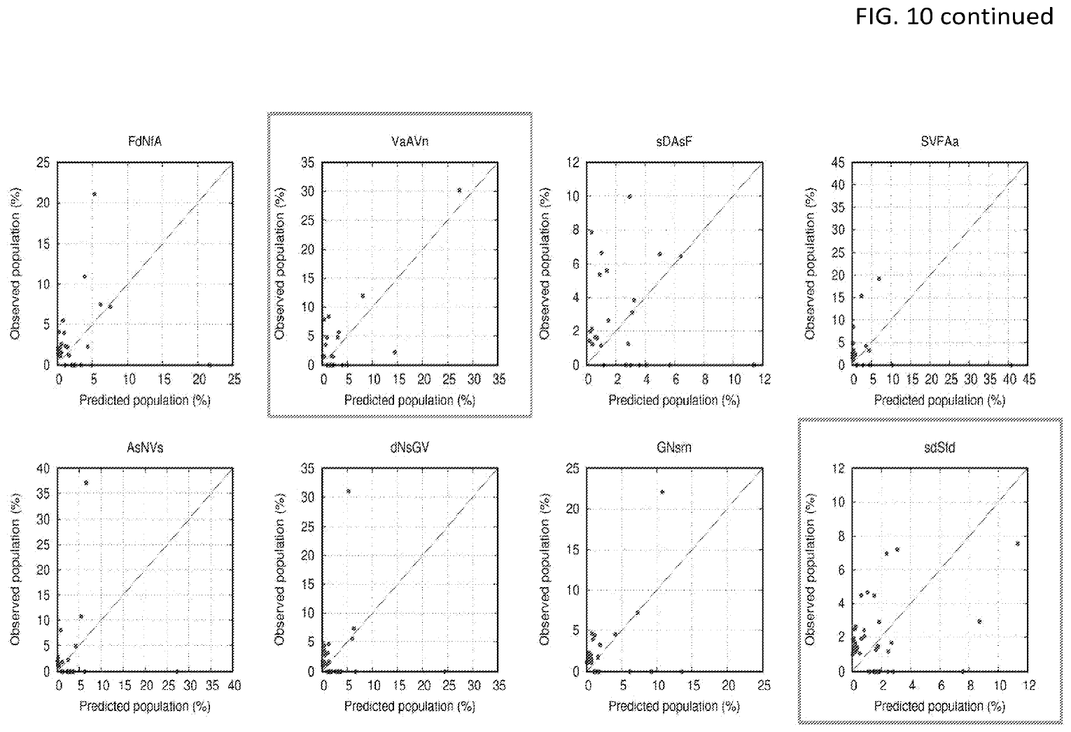

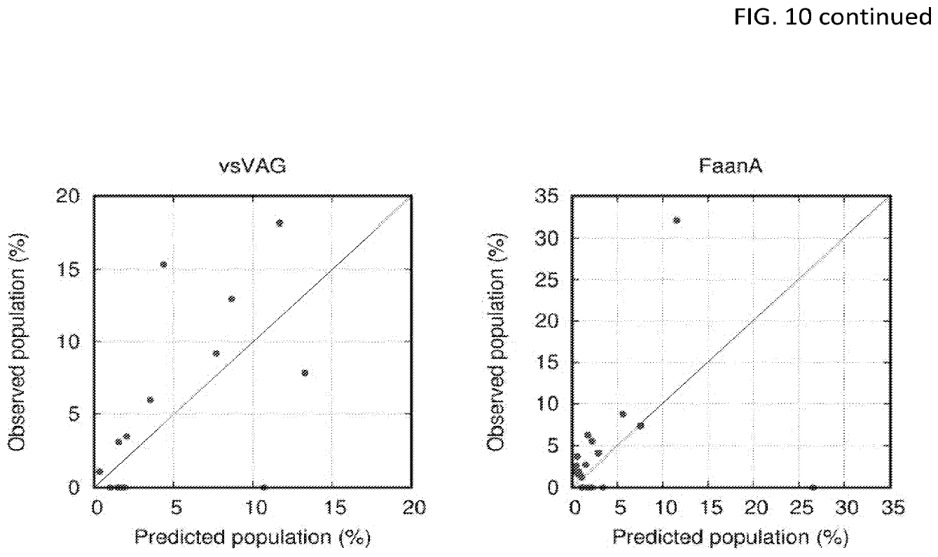

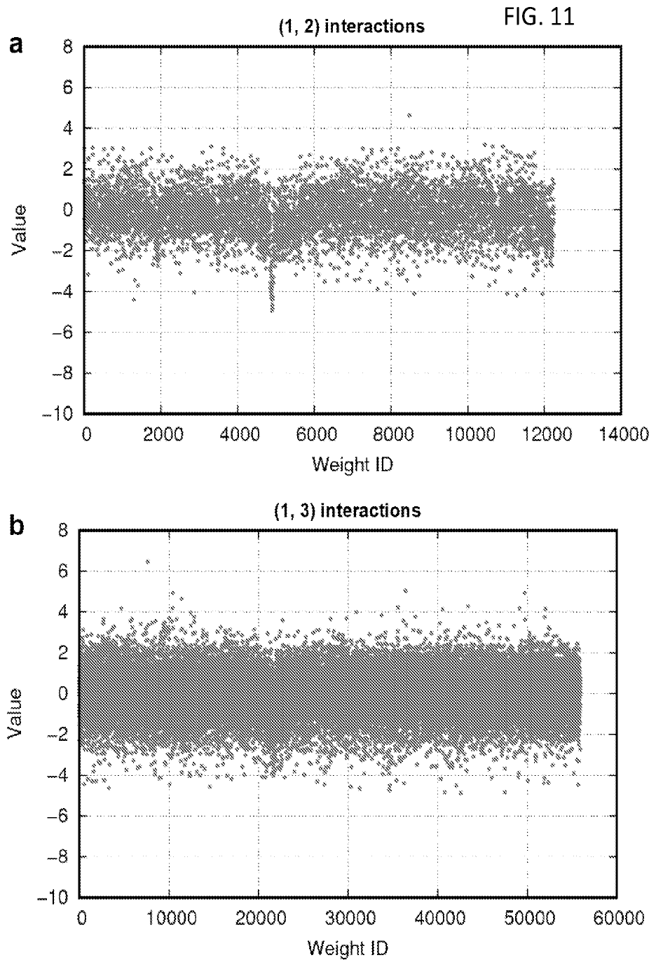

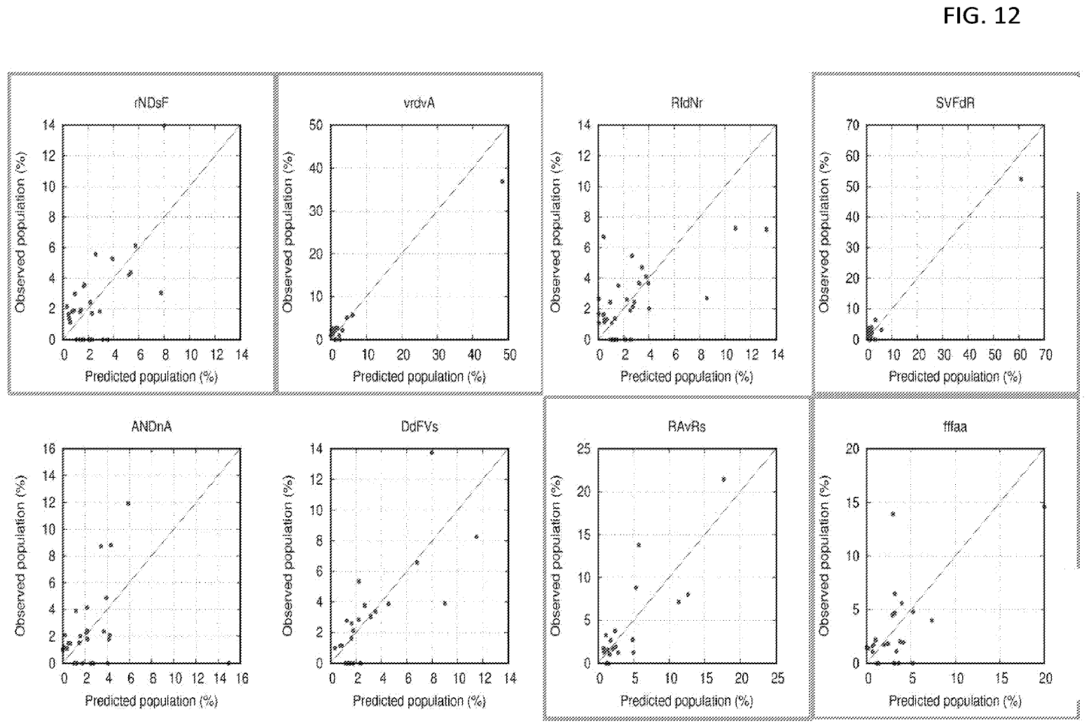

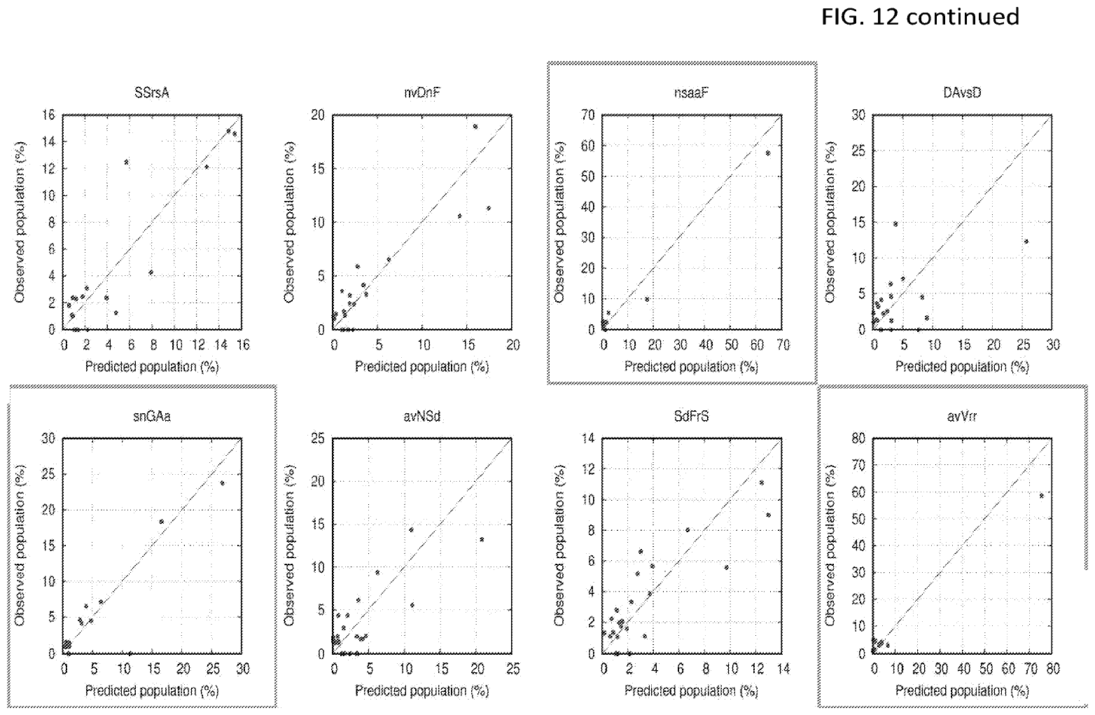

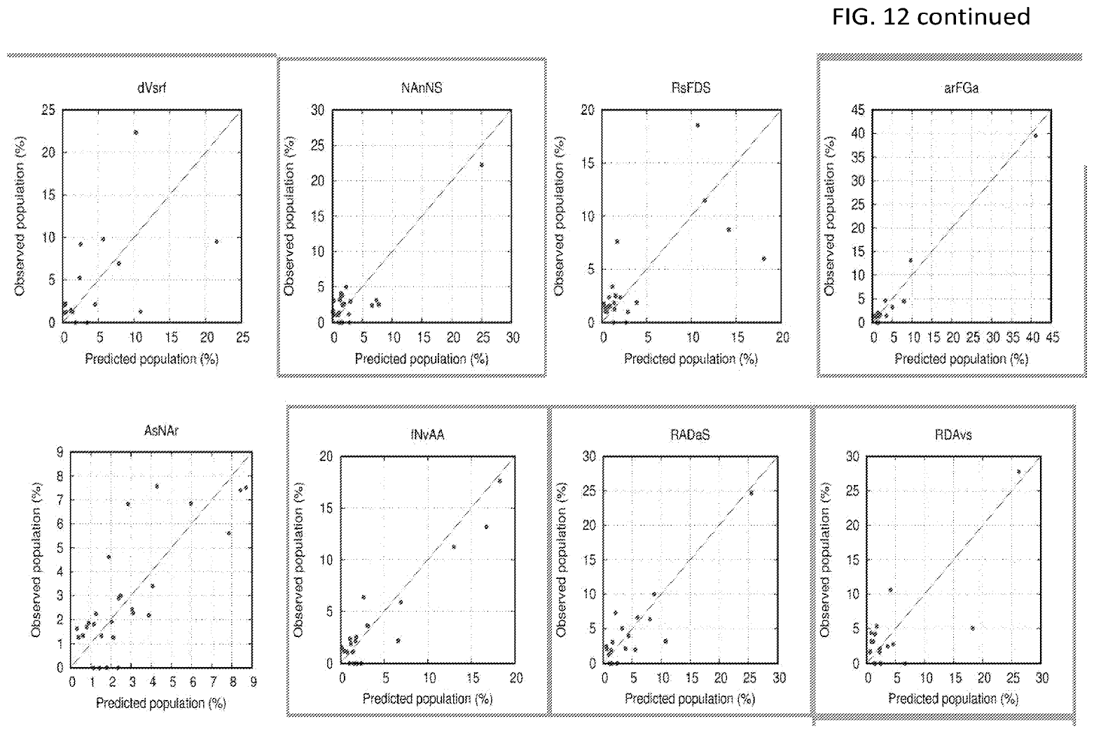

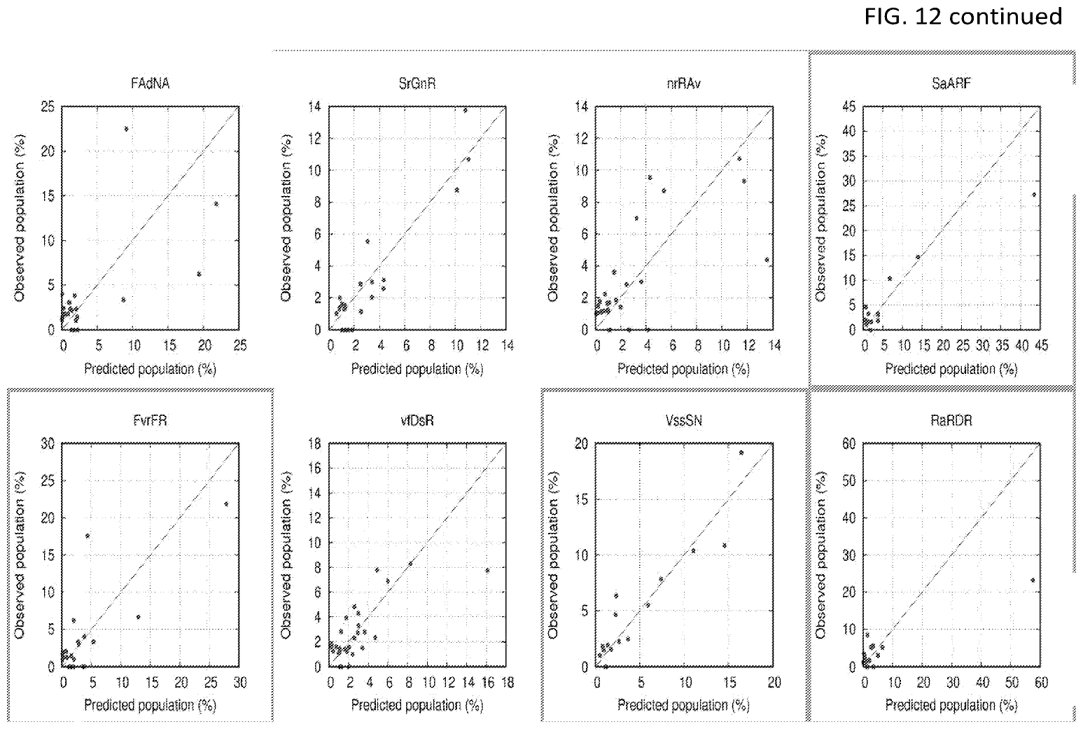

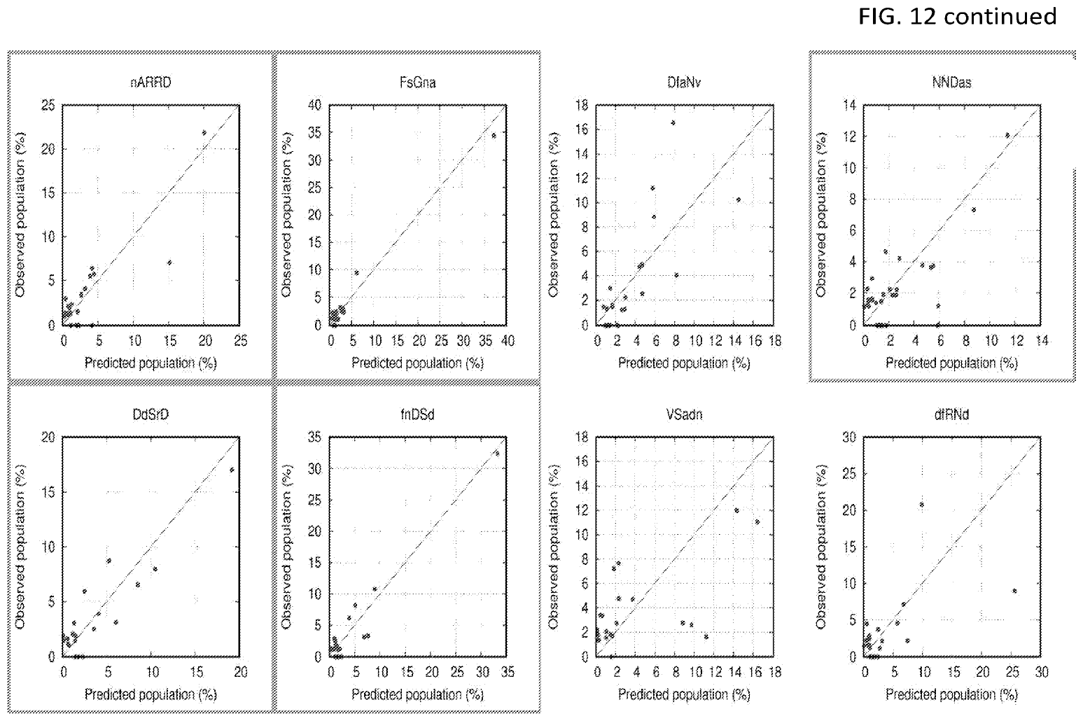

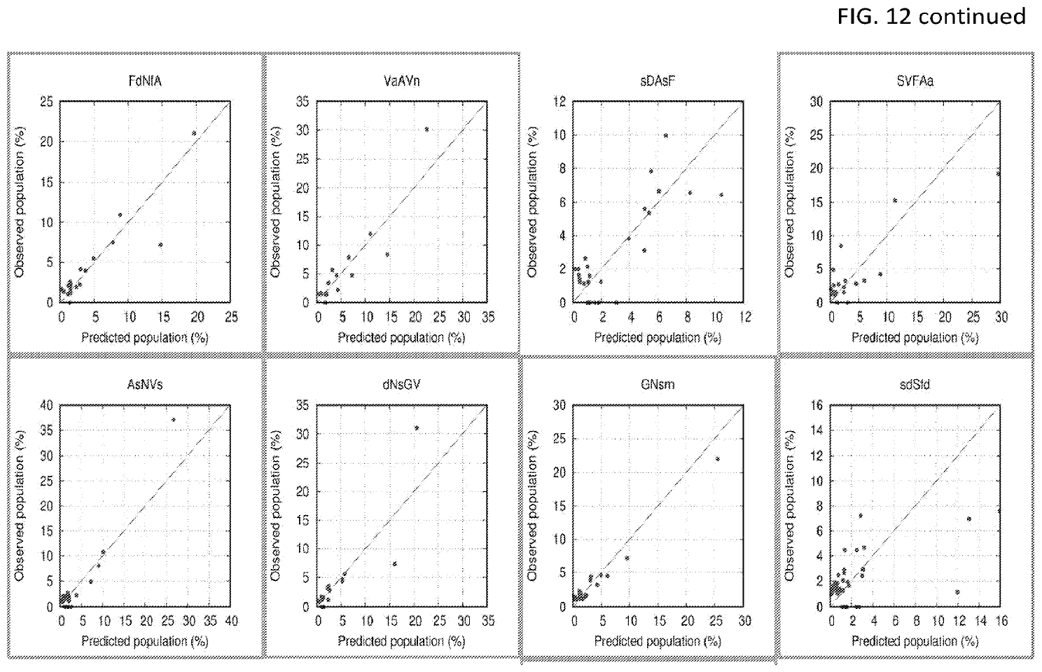

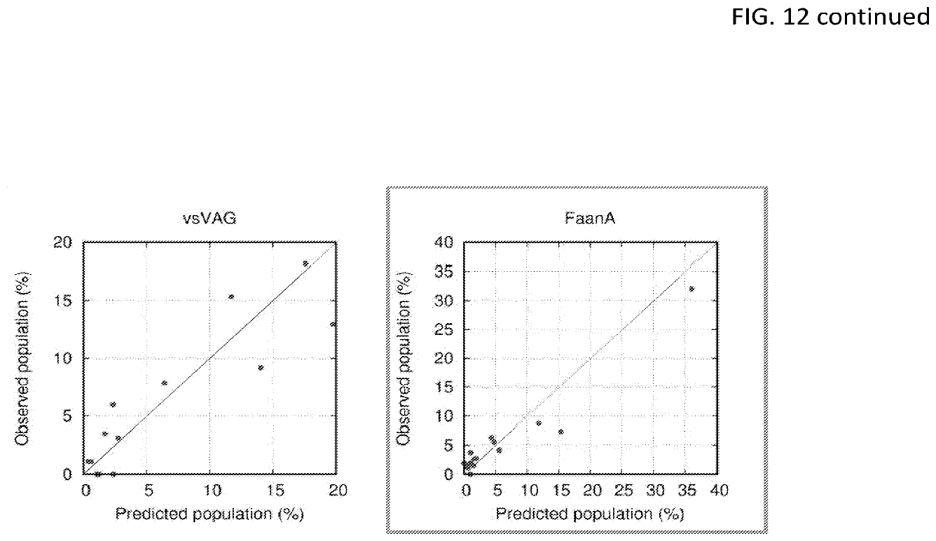

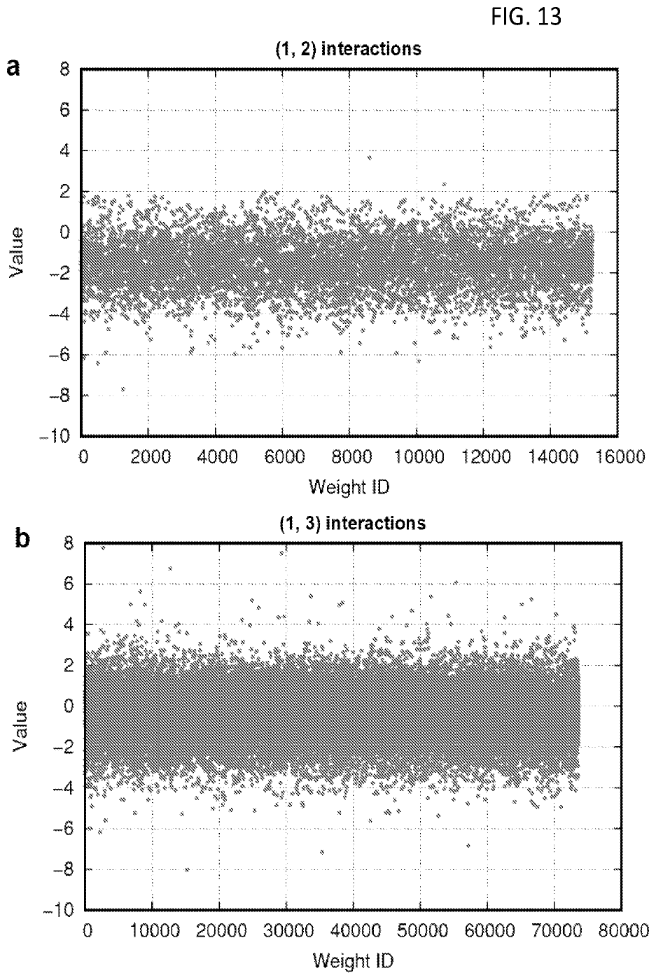

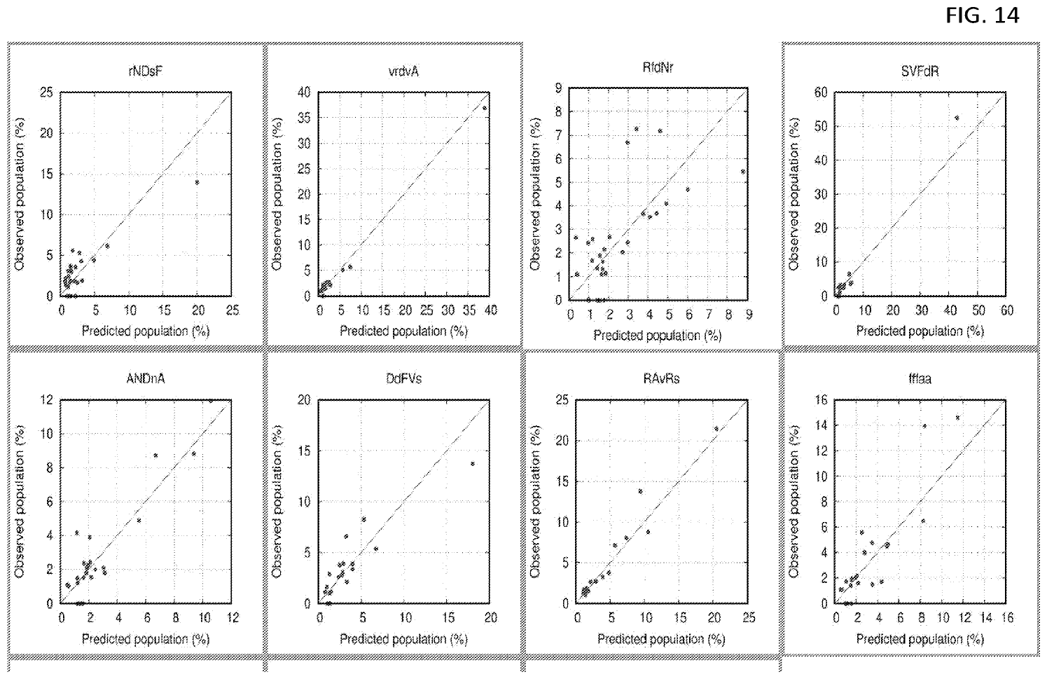

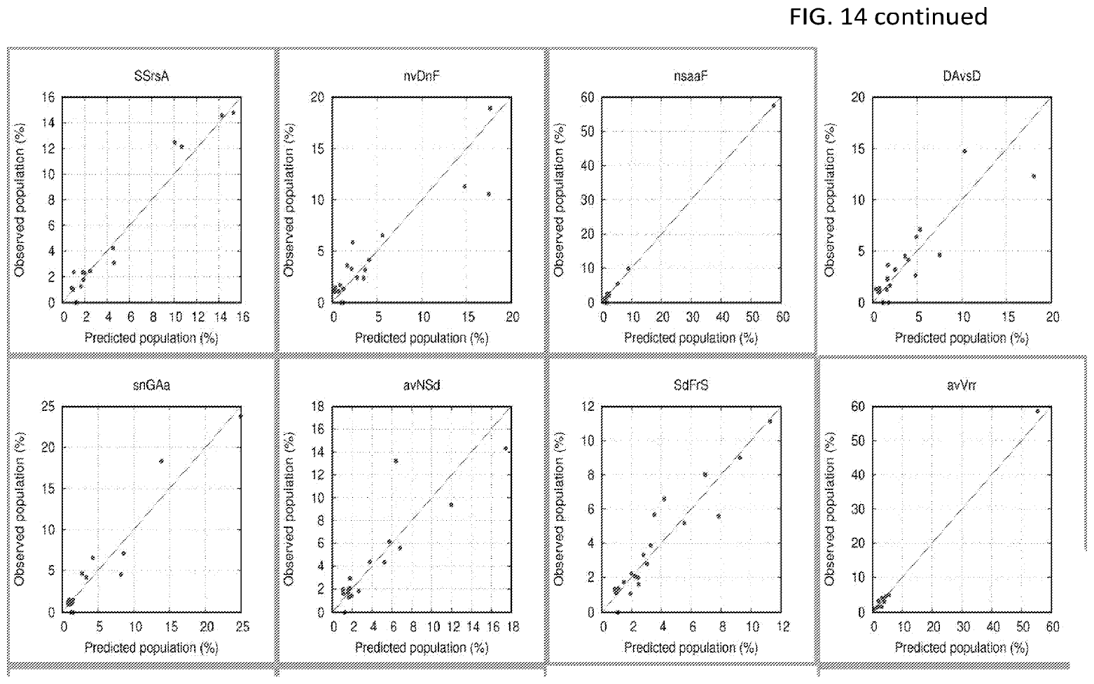

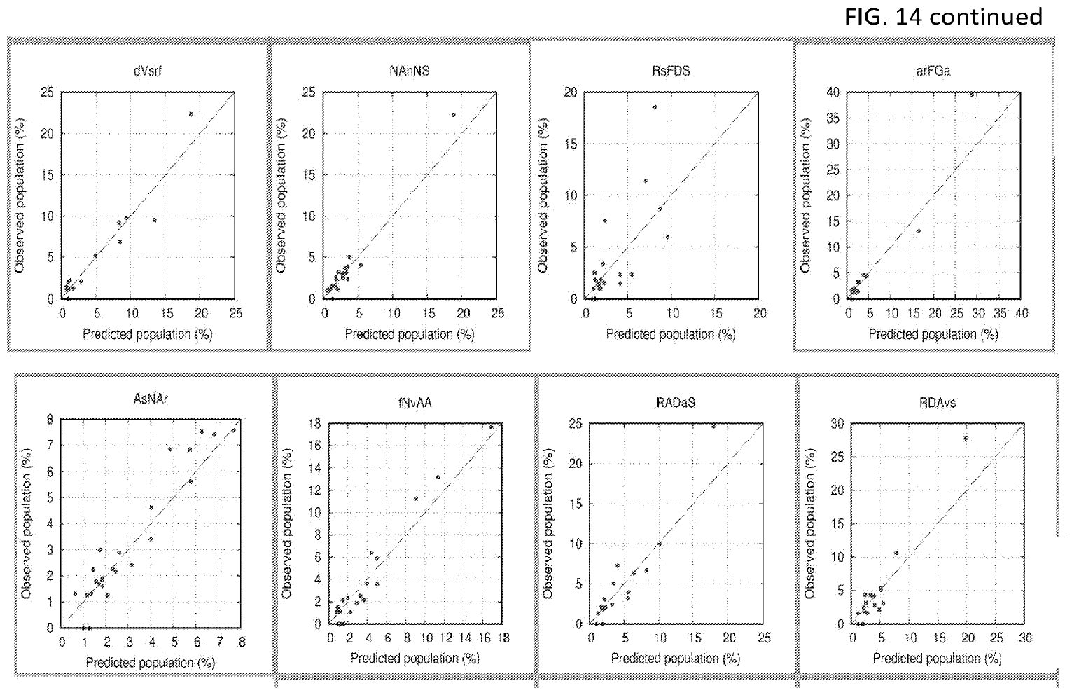

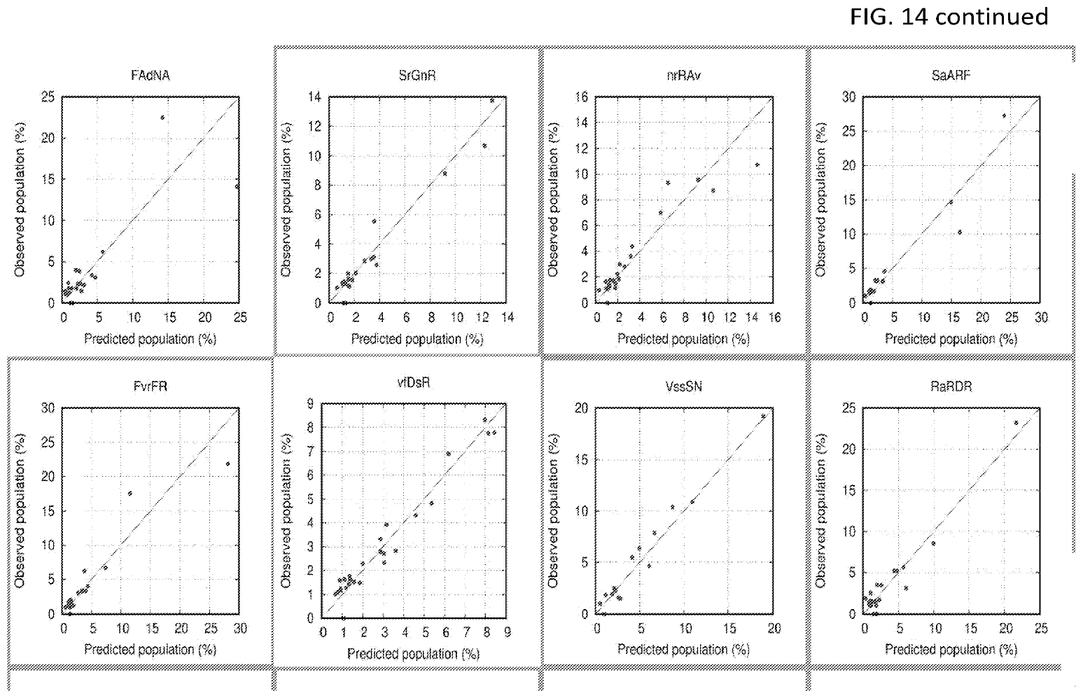

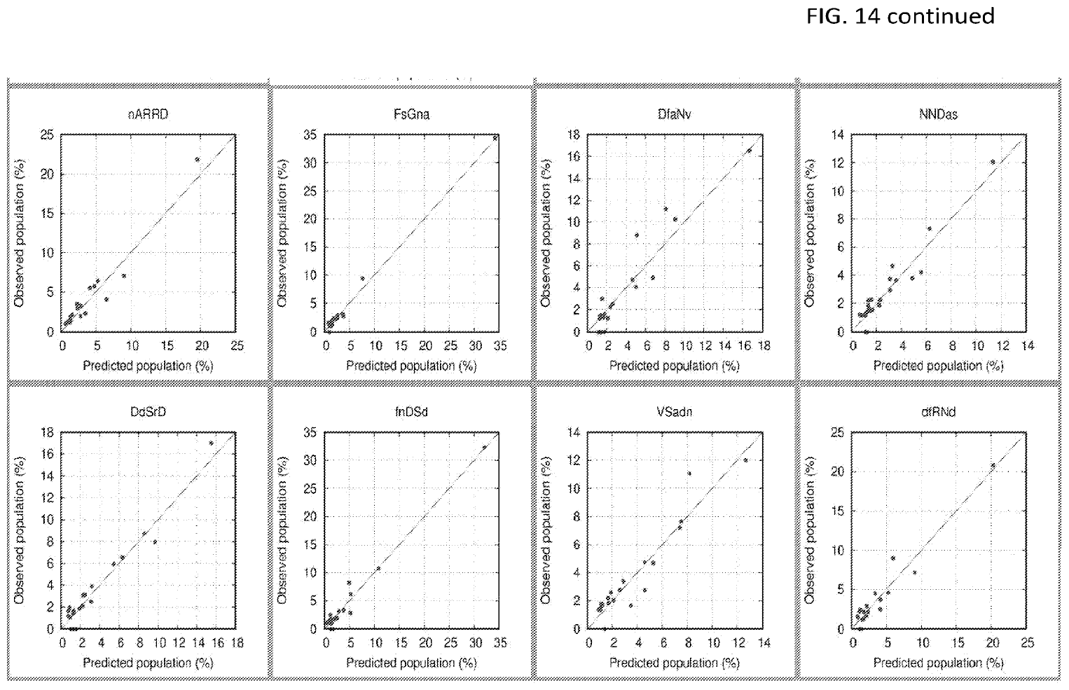

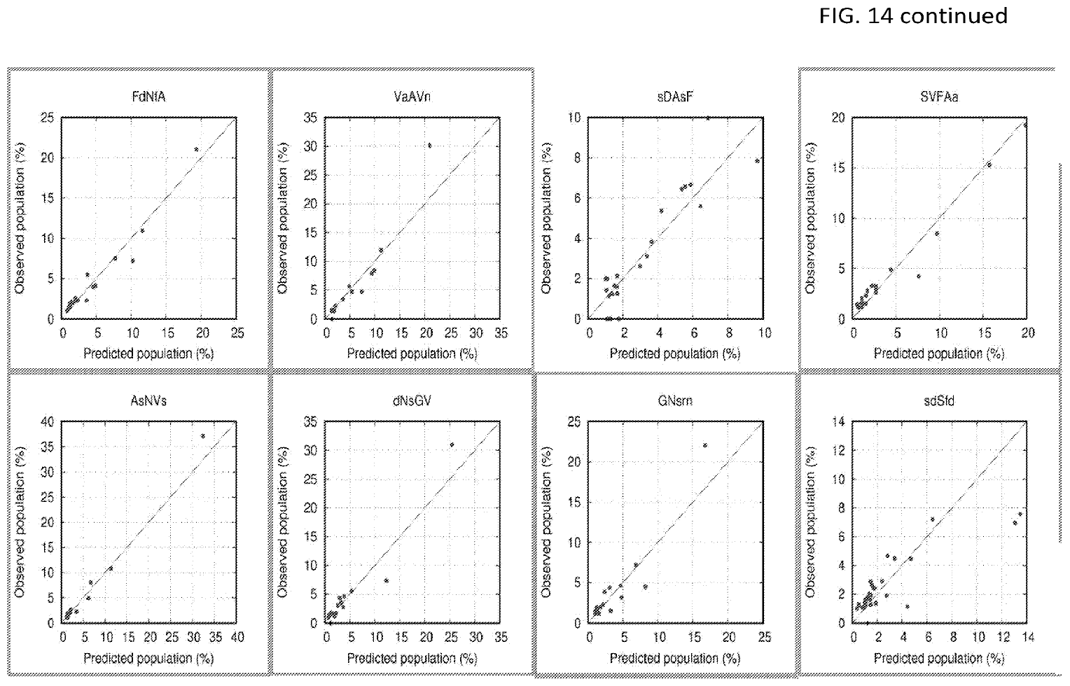

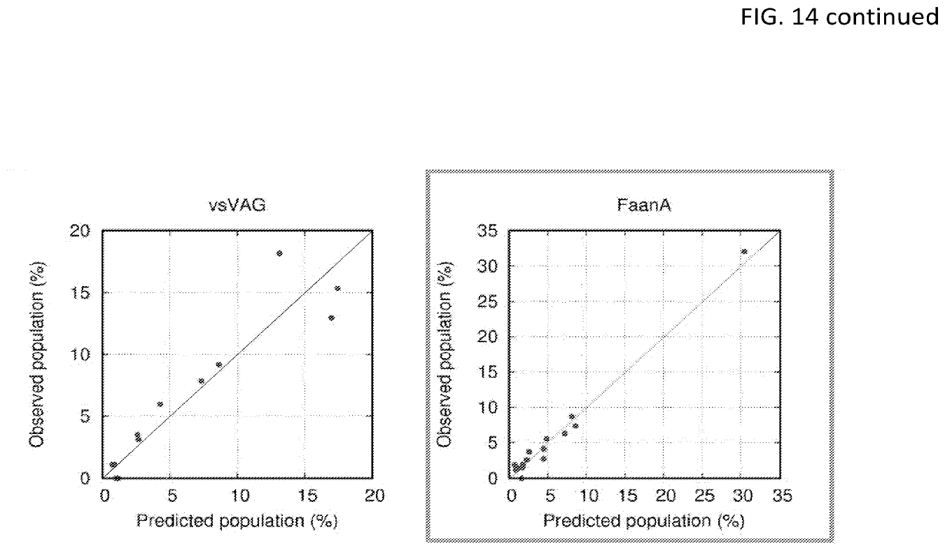

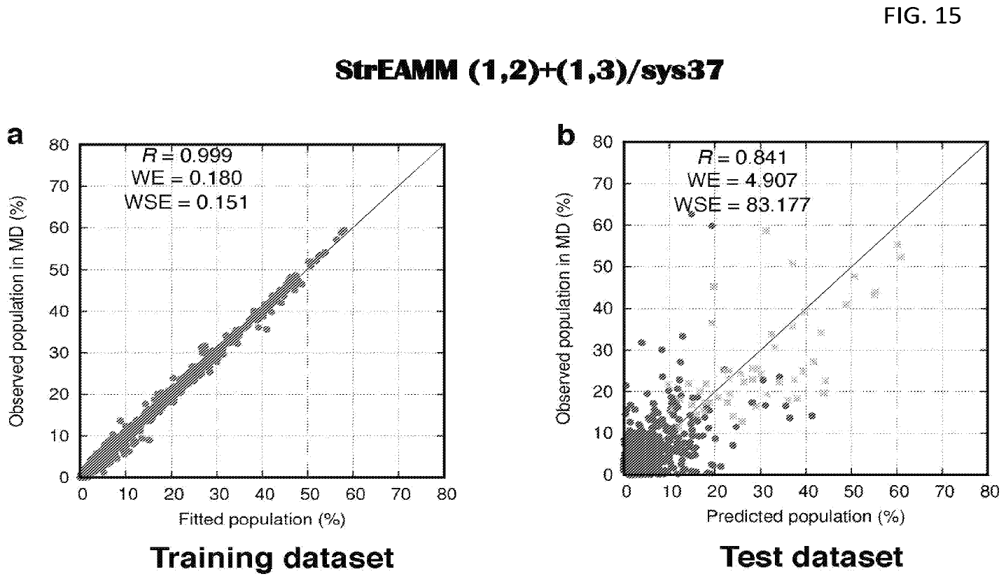

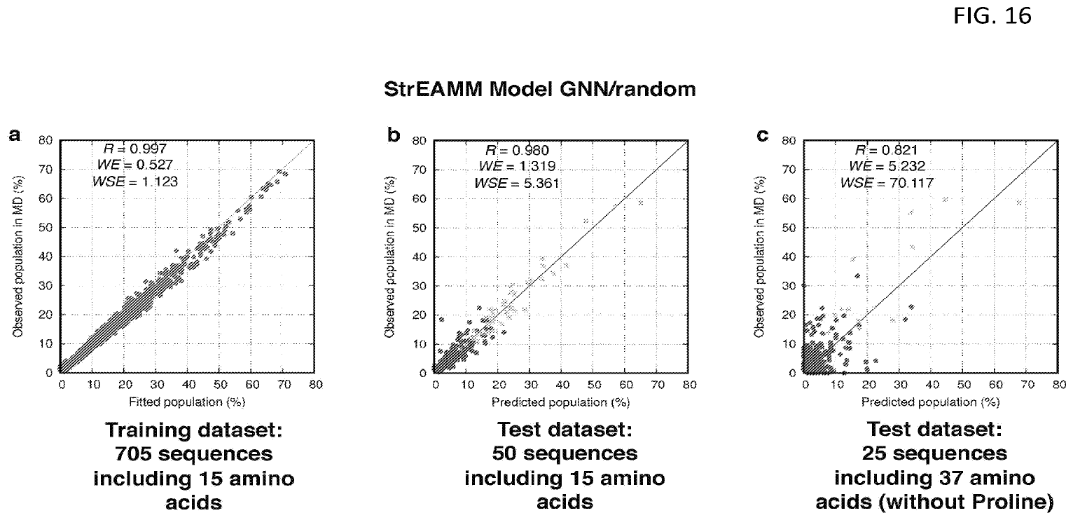

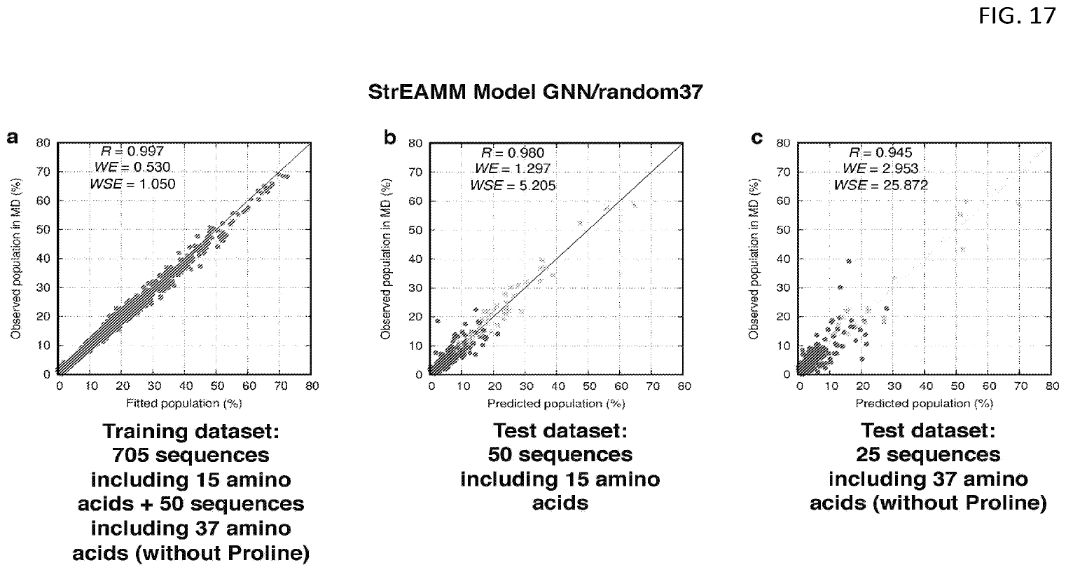

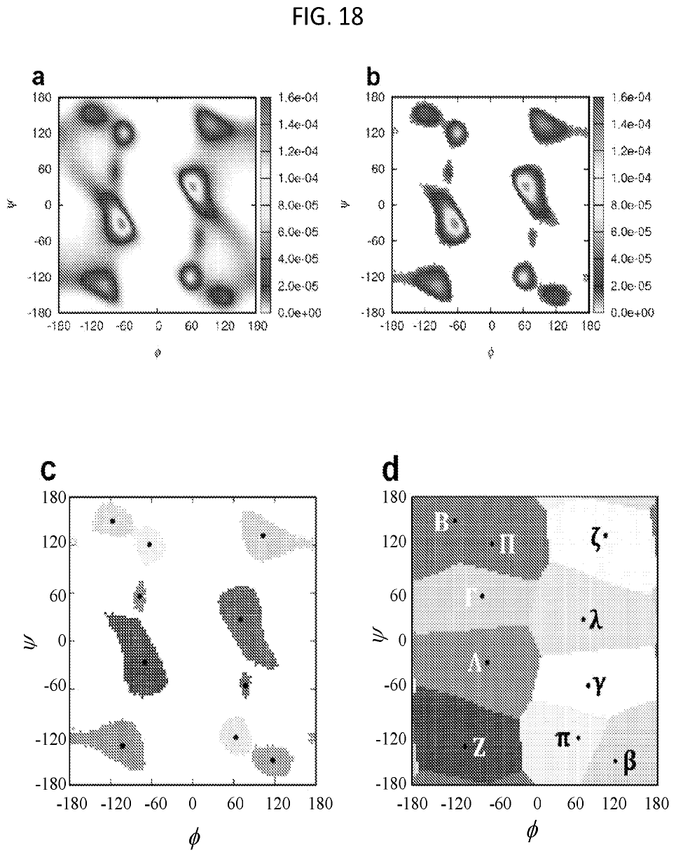

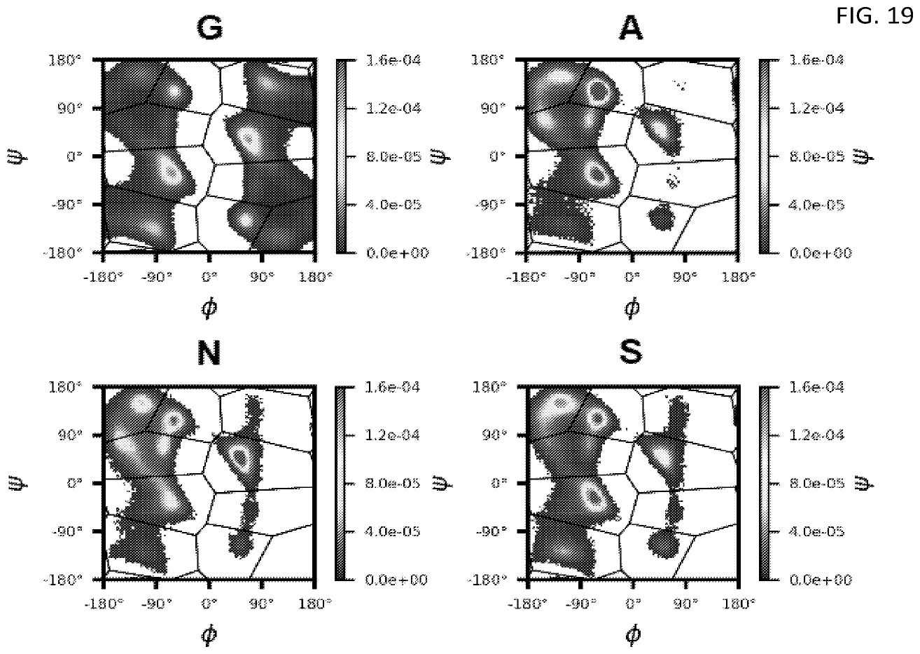

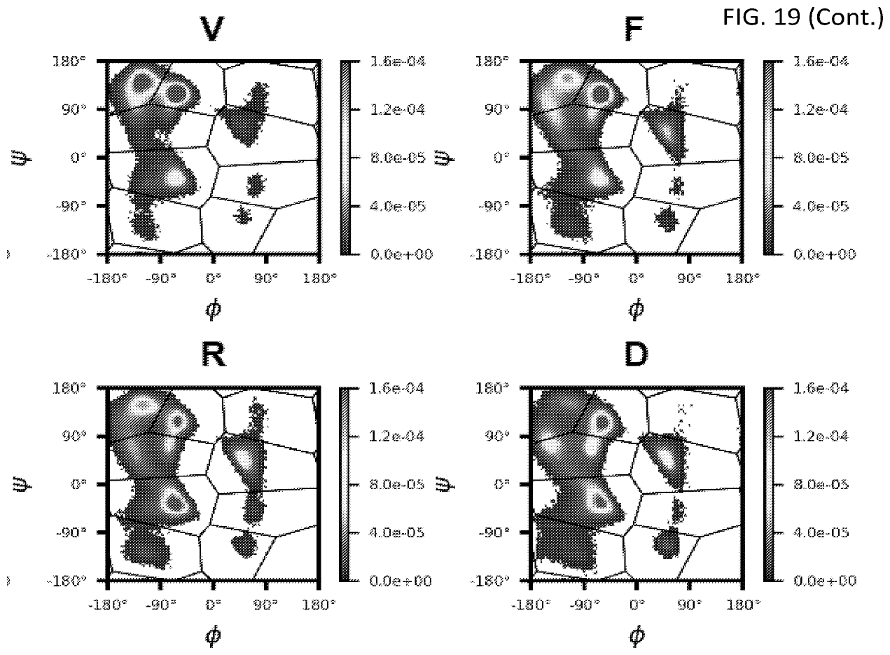

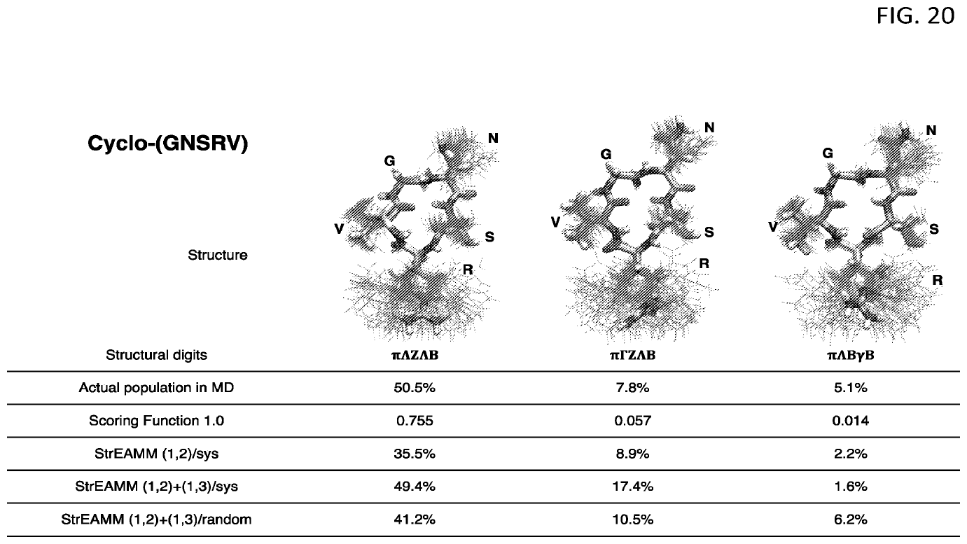

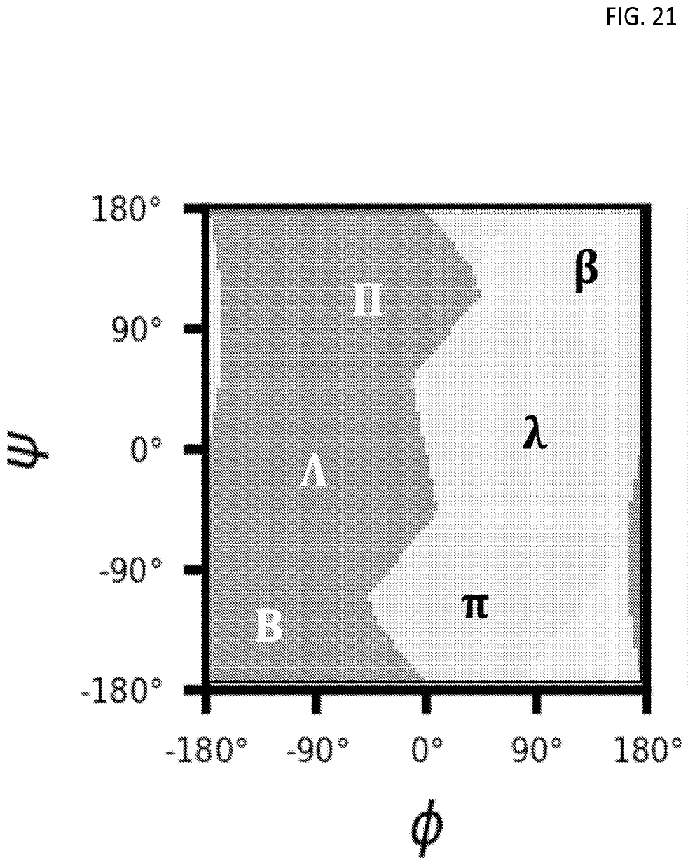

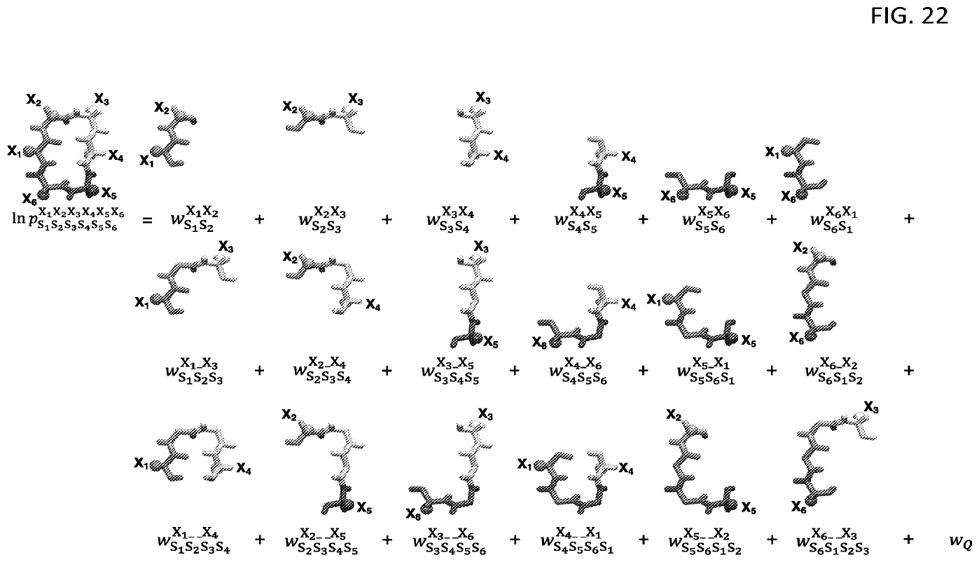

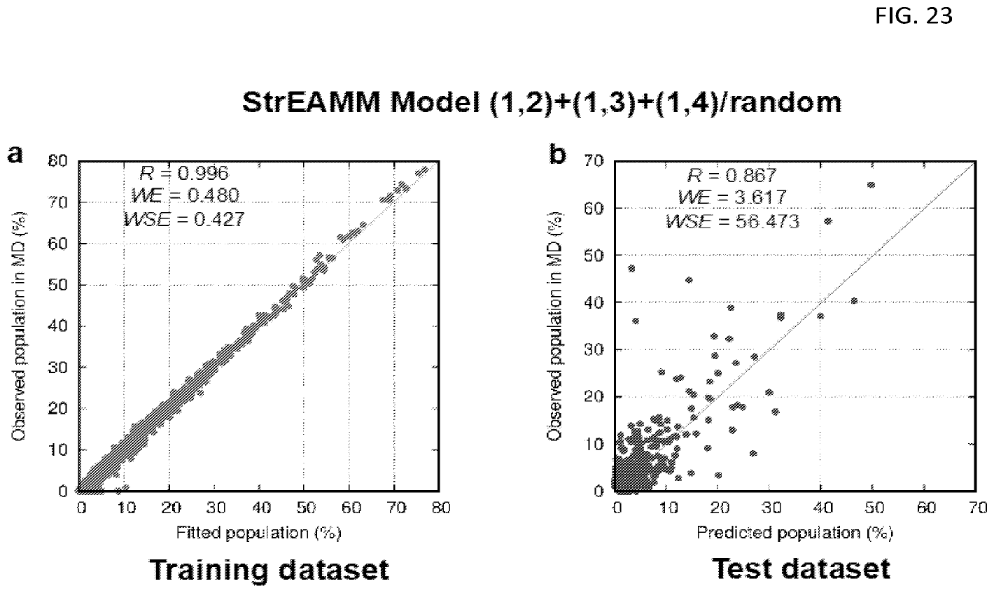

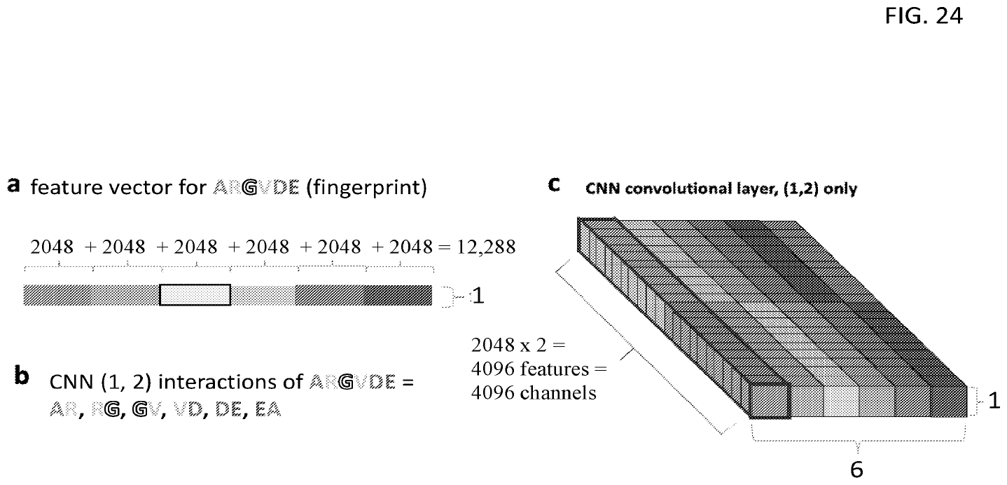

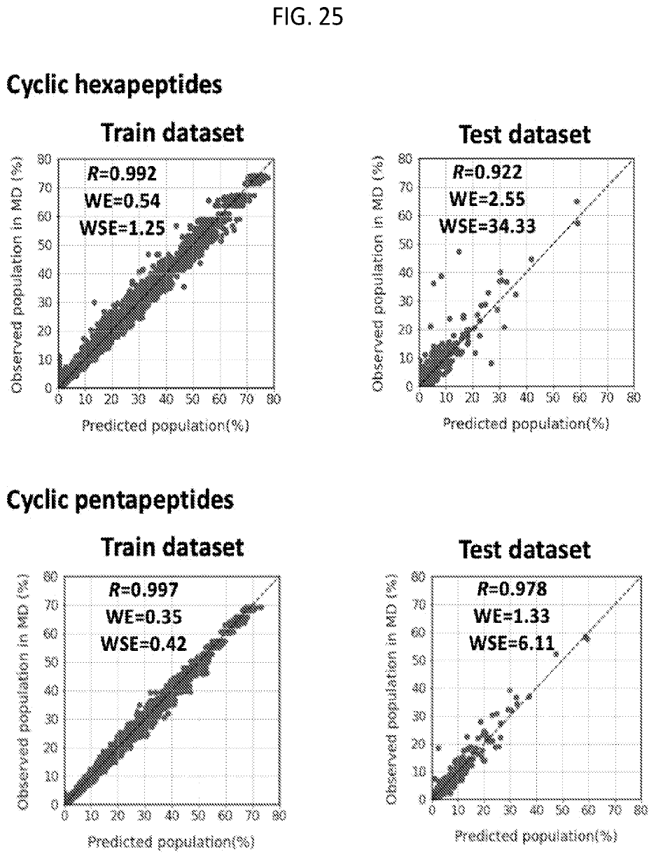

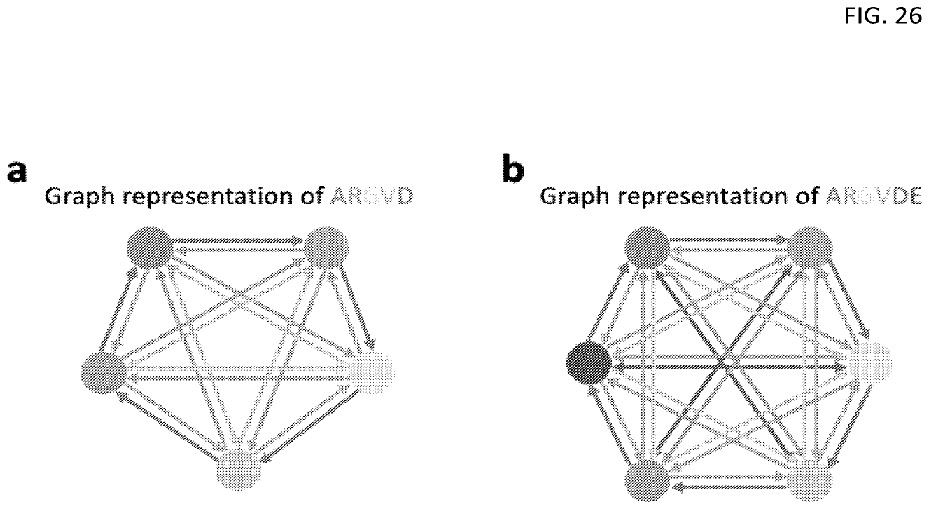

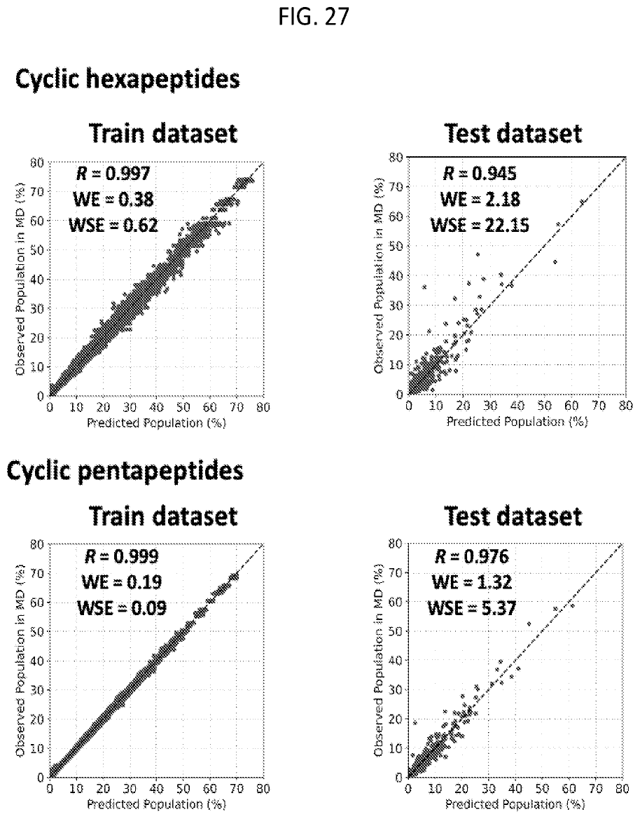

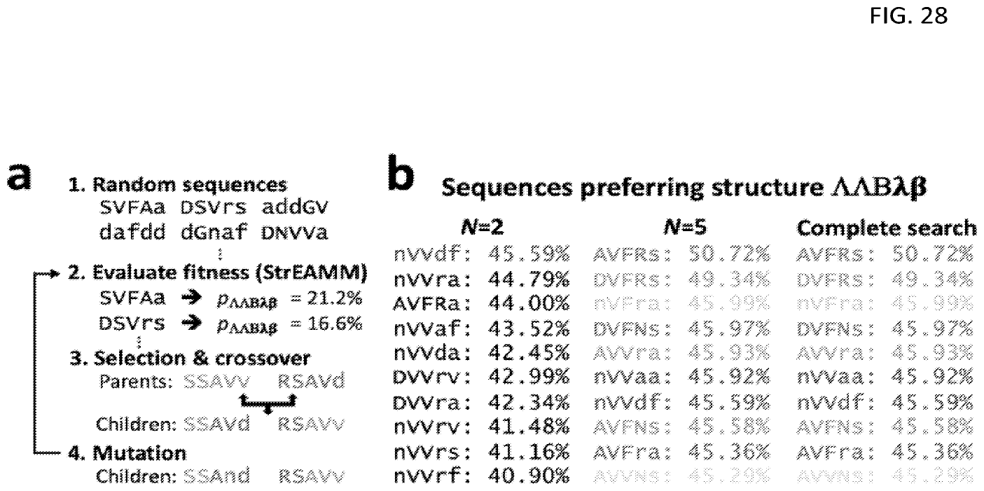

FIG. 5 a compares the fitted populations and the observed populations in the MD simulations of the training dataset (106 cyclo-(X₁X₂GGG) peptides with Xᵢ being one of 15 amino acids; see Dataset 1 in the Methods section for more detail). FIG. 5 a shows a good correlation between the fitted and observed populations. However, large deviations were observed for structures with small populations (FIG. 5 a, circle).

We then tested the performance of StrEAMM Model (1,2)/sys on 50 random cyclic-peptide sequences (Dataset 4), the same test dataset used for Scoring Function 1.0. We found the model successfully predicted the most-populated structures of 12 out of the 50 test cyclic peptides (orange stars in FIG. 5 b; also see FIG. 10, boxed), including the three well-structured cyclic peptides whose most-populated structure was larger than 50%. However, StrEAMM Model (1,2)/sys still did not perform well at predicting the full structural ensembles, especially for non-well-structured cyclic peptides, as indicated by the low Pearson correlation coefficient of 0.593 and large weighted error of 4.452 (FIG. 5 b and FIG. 4 b). This observation suggests that interactions other than nearest-neighbor (1, 2) interactions are important for determining the structural preferences of cyclic peptides and should be included in the model, or, alternatively, that the training dataset needs to be expanded.

### StrEAMM Model (1,2)+(1,3)/Sys and (1,2)+(1,3)/Random: Including Both (1,2) and (1,3) Interaction Weights

Next, we hypothesized that incorporating higher-order, longer-range contributions, specifically (1, 3) interactions, as well as nearest-neighbors (1, 2) interactions, would further enhance predictions of full structural ensembles of cyclic peptides. In this case, the population of cyclo-(X₁X₂X₃X₄X₅) adopting a specific structure S₁S₂S₃S₄S₅,

$${p}_{{S}_{1} {S}_{2} {S}_{3} {S}_{4} {S}_{5}}^{{X}_{1} {X}_{2} {X}_{3} {X}_{4} {X}_{5}}$$

was computed as:

$${p}_{{S}_{1} {S}_{2} {S}_{3} {S}_{4} {S}_{5}}^{{X}_{1} {X}_{2} {X}_{3} {X}_{4} {X}_{5}} = exp ( {w}_{{S}_{1} {S}_{2}}^{{X}_{1} {X}_{2}} + {w}_{{S}_{2} {S}_{3}}^{{X}_{2} {X}_{3}} + {w}_{{S}_{3} {S}_{4}}^{{X}_{3} {X}_{4}} + {w}_{{S}_{4} {S}_{5}}^{{X}_{4} {X}_{5}} + {w}_{{S}_{5} {S}_{1}}^{{X}_{5} {X}_{1}} + {w}_{{S}_{1} {S}_{2} {S}_{3}}^{{X}_{1 -} {X}_{3}} + {w}_{{S}_{2} {S}_{3} {S}_{4}}^{{X}_{2 -} {X}_{4}} + {w}_{{S}_{3} {S}_{4} {S}_{5}}^{{X}_{3 -} {X}_{5}} + {w}_{{S}_{4} {S}_{5} {S}_{1}}^{{X}_{4 -} {X}_{1}} + {w}_{{S}_{5} {S}_{1} {S}_{2}}^{{X}_{5 -} {X}_{2}} ) / Q . \qquad ( 16 )$$

### Here

$${w}_{{S}_{i} {S}_{i + 1}}^{{X}_{i} {X}_{i + 1}}$$

was the weight assigned when residues XᵢXᵢ₊₁ adopted structure SᵢSᵢ₊₁;

$${w}_{{S}_{i} {S}_{i + 1} {S}_{i + 2}}^{{X}_{i -} {X}_{i + 2}}$$

was the weight assigned when residues Xᵢ₋Xᵢ₊₂ adopted structure SᵢSᵢ₊₁Sᵢ₊₂. Note that in describing (1, 3) interactions, we also included the structural digit of the middle residue. This decision recognized that the (ϕ, ψ) dihedrals of the middle residue would likely affect the relative distance and orientation between residues Xᵢ and Xᵢ₊₂. However, the description did not consider the identity of the amino acid at middle residue Xᵢ₊₁, only the structural digit. The expression (in the logarithmic form) is illustrated in FIG. 2 c. The weights were then determined by weighted least square fitting to minimize the difference between the predicted populations and the actual populations observed in the MD simulations of the training sequences.

To train the weights related to both (1, 2) and (1, 3) interactions, we devised two training datasets. The first training dataset included 204 cyclo-(X₁X₂GGG) and cyclo-(X₁ GX₃GG) peptides (see Dataset 2 in the Methods section for more detail), and the resulting model was termed StrEAMM Model (1,2)+(1,3)/sys. The second training dataset included 705 cyclo-(X₁X₂X₃X₄X₅) peptides of semi-random sequences that ensured all X₁X₂X₃ patterns were observed and each X₁X₂ and X₁₋X₃ patterns appeared at least 15 times (see Dataset 3 in the Methods section for more detail); the resulting model was termed StrEAMM Model (1,2)+(1,3)/random.

FIG. 5 c compares the observed populations in MD simulations and the fitted populations from StrEAMM Model (1,2)+(1,3)/sys for the training dataset in Dataset 2. FIG. 5 e compares the observed populations in MD simulations and the fitted populations from StrEAMM Model (1,2)+(1,3)/random for the training dataset in Dataset 3. The results from both models show a clear correlation between the fitted and the observed populations.

We then tested StrEAMM Models (1,2)+(1,3)/sys and (1,2)+(1,3)/random on 50 random cyclic-peptide sequences in Dataset 4, the same test dataset used for Scoring Functions 1.0 and StrEAMM Model (1,2)/sys. For both StrEAMM Models (1,2)+(1,3)/sys and (1,2)+(1,3)/random (FIGS. 5 d and 5f), the correlation between the observed populations in MD simulations and predicted populations was much improved over Scoring Function 1.0 (FIG. 3) and StrEAMM Model (1,2)/sys (FIG. 5 b). StrEAMM Model (1,2)+(1,3)/sys successfully predicted the most-populated structures of 30 of the 50 test cyclic peptides (orange stars in FIG. 5 d; also see FIG. 12, boxed in green), and the Pearson correlation coefficient was 0.912 when comparing the predicted and the observed populations. The weighted error also dropped to 2.972. The results were even more impressive for StrEAMM Model (1,2)+(1,3)/random, which successfully predicted the most-populated structures of 43 of the 50 test cyclic peptides (orange stars in FIG. 5 f; also see FIG. 14, boxed in green). The Pearson correlation coefficient was 0.974 between the predicted and the observed populations. The weighted error was 1.543. FIG. 4 shows that StrEAMM Model (1,2)+(1,3)/random not only described the structural ensemble of the well-structured cyclo-(avVrr), but also successfully predicted the structural ensemble of the non-well-structured cyclo-(SVFAa). In fact, StrEAMM Model (1,2)+(1,3)/random consistently predicted the structural ensemble even for cyclic peptides whose most-populated structure represented as little as 10% of the total ensemble.

### Experimental Evaluation

In the work of Slough et al.²⁴, cyclo-(GNSRV) (SEQ ID NO: 51) was predicted to be a well-structured cyclic peptide. However, in their work, they could not predict the exact population. The comparison between the prediction of StrEAMM models and the MD simulation results are shown in FIG. 20. The predicted populations by StrEAMM models (1,2)+(1,3)/sys and (1,2)+(1,3)/random are close to the observed populations in the MD simulations. The two structures πΛZΛB and πΓZΔB with the most and the second most populations correspond to a type II′ β turn at ¹GN² and an α_{R} tight turn at R⁴, which was supported by NMR experiments. (Slough et al.)

### Extendibility of StrEAMM Model: Graph Neural Networks (GNNs) and Amino-Acid Fingerprints

More advanced neural networks and amino-acid representations can be introduced to the StrEAMM model. Here we provide such an example and show the extendibility of the model. In this example, we trained a GNN (message passing network) to predict structural ensembles of cyclic pentapeptides while encoding the peptides as a graph. GNNs have been applied to chemical systems due to their potential to handle inputs of diverse graph structures.

Neural network training and graph creation were done using Pytorch 1.9.08 and Pytorch Geometric 1.7.2.⁹ Amino acids were encoded using circular topological molecular fingerprints, specifically Morgan Fingerprints¹⁰ generated with RDKit version 2021.03.05,¹¹ using a radius of three and a fingerprint length of 2048 bits; amino acids were input with NH₂ and COOH termini, and sidechain charges matched the charges used in the MD simulations. With this encoding, every amino acid in a cyclic-peptide sequence can be represented by a 2048-bit fingerprint. To represent the structural ensemble of a cyclic peptide, we used an array of 2742 populations where each population in the array corresponded to a structure or a cyclic permutation of a structure in the structure pool (List 1). We note that there are fewer than 2750 (550×5) populations because “ΛΛΛΛΛ” and “λλλλλ” in the structure pool are cyclic invariant.

In preparation for the use of a GNN, we represented a cyclic pentapeptide as a graph with one node for each amino acid in the sequence and the initial node representation given by an amino acid's molecular fingerprint. Nodes were connected by four types of directed edges. Two types of edges (forward and backward with respect to peptide sequence) connected (1, 2) neighbor nodes, and two types of edges connected (1, 3) neighbor nodes. The edges must be directed to prevent a sequence and its retroisomer (reverse ordering sequence) from being encoded as identical graphs. Thus, a cyclic pentapeptide is represented by a graph with 5 nodes and 20 edges.

We constructed a GNN that converted a cyclic-pentapeptide graph into an array of structure populations. The network performed the following sequence of operations. From the input graph, we performed one message passing operation using the RGCNConv operator through Pytorch Geometric.¹⁴ This operator updated a node representation in the graph by summing up the node's transformed initial representation and transformed representations of the node's (1, 2) and (1, 3) neighbors. Each different edge type had a unique learned transformation. A rectified linear unit (ReLU) activation function was then applied to the node representations. Next, the node representations were concatenated and transformed by a two dense layer of 2048 nodes into a structural ensemble represented by an array of 2742 populations with a ReLU activation function on the dense layer, and a softmax activation function on the final layer to ensure the output structural ensemble was normalized.

The models were trained using the Adam optimizer and summation of the squared errors loss function

$$( L = {\sum }_{i = 1}^{N} {( {p}_{i , learned} - {p}_{i} )}^{2} ,$$

where N is the number of populations in the training dataset, p_{i,learned} is the learned population by the network, pᵢ is the actual population observed in MD simulations) for 1000 epochs with a learning rate of 0.000005 and a batch size of 50. To account for the non-cyclic permutation invariant operation of node concatenation, we trained on all cyclic permutations of a sequence, as well as the corresponding enantiomer sequences, whose data we constructed from the initial simulation results of a sequence by cyclically permuting structural digits or flipping them across the centro-symmetric structural map for the two different cases respectively. By doing this, we aimed to train the model to be invariant to cyclic permutations of the input sequence. The first model was trained on the semi-randomly generated Dataset 3 containing 15 types of representative amino acids, as well as their cyclically permuted sequences and enantiomer sequences (7050 input graphs). We call this model StrEAMM GNN/random hereafter. The second model was trained on Dataset 3 and 50 additional random sequences containing 37 types of amino acids (Dataset 6.1, List 5), as well as their cyclically permuted sequences and enantiomers (7550 input graphs). We call this model StrEAMM GNN/random37 hereafter.

To evaluate the performance of the models, we tested them on the 50 sequences of Dataset 4 that contain 15 types of representative amino acids (List 2) and on the 25 sequences of Dataset 6.2 that contain 37 types of amino acids (List 5). The results for StrEAMM GNN/random and StrEAMM GNN/random37 are shown in FIGS. 16 and 17, respectively. We see that StrEAMM GNN/random, StrEAMM GNN/random37, and StrEAMM (1,2)+(1,3)/random produced comparable good predictions for the 50 15-amino-acid sequences in Dataset 4. Moreover, after introducing the fingerprint encodings, StrEAMM GNN/random was able to predict the structural ensembles of sequences composed by amino acids not contained in the training dataset with reasonable accuracy (with Pearson correlation coefficient of 0.821 and a weighted error of 5.23%; FIG. 16). Results of StrEAMM GNN/random37 showed that the performance of the model could be further improved by including only 50 additional sequences that contain 37 types of amino acids (Pearson correlation coefficient was increased to 0.945, and the weighted error was reduced to 2.95%; FIG. 17). These results indicate that the StrEAMM model is readily extendible to amino acids beyond the 15 representative types.

### Binning the Ramachandran Plot

The Ramachandran plot of cyclo-(GGGGG) (SEQ ID NO: 83) was first divided into 100×100 grids, and the probability density of each grid was calculated (FIG. 18 a). Cluster analysis was only performed on the grids with a probability density larger than 0.00001 (FIG. 18 b) using a grid-based and density peak-based method. 15 FIG. 18 c shows the resulting 10 clusters. The centroid of each cluster was determined as the grid point with the smallest average of distances weighted by probability density to the remaining grids of the cluster (FIG. 18 c, black dots). All the other grid points in the Ramachandran plot were then assigned to their closest centroid (FIG. 18 d) to obtain the final map. To verify the applicability of the binning map to non-Gly residues, FIG. 19 shows the Ramachandran plot of the first residue in cyclo-(GGGGG) (SEQ ID NO: 83), cyclo-(AGGGG) (SEQ ID NO: 84), cyclo-(VGGGG) (SEQ ID NO: 85), cyclo-(FGGGG) (SEQ ID NO: 86), cyclo-(NGGGG) (SEQ ID NO: 87), cyclo-(SGGGG) (SEQ ID NO: 88), cyclo-(RGGGG) (SEQ ID NO: 89), and cyclo-(DGGGG) (SEQ ID NO: 90), with the boundaries of the map shown. The binning map is capable of separating the major peaks in these Ramachandran plots as well.

### Linear StrEAMM Model for Cyclic Hexapeptides

Fifteen representative amino acids were used in this study: G, A, V, F, N, S, D, R, a, v, f, n, s, d, and r, with lowercase letters denote D-amino acids. The binning map used to bin the backbone dihedrals is shown in FIG. 21.

The linear StrEAMM (1,2)+(1,3)+(1,4) model incorporates (1,2), (1,3) and (1,4) interactions into the model.

The population of cyclo-(X₁X₂X₃X₄X₅X₆) adopting a specific structure S₁S₂S₃S₄S₅S₆,

$${p}_{{S}_{1} {S}_{2} {S}_{3} {S}_{4} {S}_{5} {S}_{6}}^{{X}_{1} {X}_{2} {X}_{3} {X}_{4} {X}_{5} {X}_{6}}$$

was computed as:

$${p}_{{S}_{1} {S}_{2} {S}_{3} {S}_{4} {S}_{5} {S}_{6}}^{{X}_{1} {X}_{2} {X}_{3} {X}_{4} {X}_{5} {X}_{6}} = exp ( {w}_{{S}_{1} {S}_{2}}^{{X}_{1} {X}_{2}} + {w}_{{S}_{2} {S}_{3}}^{{X}_{2} {X}_{3}} + {w}_{{S}_{3} {S}_{4}}^{{X}_{3} {X}_{4}} + {w}_{{S}_{4} {S}_{5}}^{{X}_{4} {X}_{5}} + {w}_{{S}_{5} {S}_{6}}^{{X}_{5} {X}_{6}} + {w}_{{S}_{6} {S}_{1}}^{{X}_{6} {X}_{1}} + {w}_{{S}_{1} {S}_{2} {S}_{3}}^{{X}_{1 -} {X}_{3}} + {w}_{{S}_{2} {S}_{3} {S}_{4}}^{{X}_{2 -} {X}_{4}} + {w}_{{S}_{3} {S}_{4} {S}_{5}}^{{X}_{3 -} {X}_{5}} + {w}_{{S}_{4} {S}_{5} {S}_{6}}^{{X}_{4 -} {X}_{6}} + {w}_{{S}_{5} {S}_{6} {S}_{1}}^{{X}_{5 -} {X}_{1}} + {w}_{{S}_{6} {S}_{1} {S}_{2}}^{{X}_{6 -} {X}_{2}} + {w}_{{S}_{1} {S}_{2} {S}_{3} {S}_{4}}^{{X}_{1 --} {X}_{4}} + {w}_{{S}_{2} {S}_{3} {S}_{4} {S}_{5}}^{{X}_{2 --} {X}_{5}} + {w}_{{S}_{3} {S}_{4} {S}_{5} {S}_{6}}^{{X}_{3 --} {X}_{6}} + {w}_{{S}_{4} {S}_{5} {S}_{6} {S}_{1}}^{{X}_{4 --} {X}_{1}} + {w}_{{S}_{5} {S}_{6} {S}_{1} {S}_{2}}^{{X}_{5 --} {X}_{2}} + {w}_{{S}_{6} {S}_{1} {S}_{2} {S}_{3}}^{{X}_{6 --} {X}_{3}} ) / Q$$

### Here

$${w}_{{S}_{i} {S}_{i + 1}}^{{X}_{i} {X}_{i + 1}}$$

was the weight assigned when residues XᵢXᵢ₊₁ adopted structure SᵢSᵢ₊₁;

$${w}_{{S}_{i} {S}_{i + 1} {S}_{i + 2}}^{{X}_{i -} {X}_{i + 2}}$$

was the weight assigned when residues Xᵢ₋Xᵢ₊₂ adopted structure SᵢSᵢ₊₁Sᵢ₊₂;

$${w}_{{S}_{i} {S}_{i + 1} {S}_{i + 2} {S}_{i + 3}}^{{X}_{i -} {X}_{i + 3}}$$

was the weight assigned when residues Xᵢ₋₋Xᵢ₊₃ adopted structure SᵢSᵢ₊₁Sᵢ₊₂Sᵢ₊₃. Note that in describing (1, 3) and (1, 4) interactions, we also included the structural digit(s) of the middle residue(s). This decision recognized that the (ϕ, ψ) dihedrals of the middle residue(s) would likely affect the relative distance and orientation between the two residues at the ends (Xᵢ and Xᵢ₊₂ for (1, 3) interactions, Xᵢ and Xᵢ₊₃ for (1, 4) interactions). However, the description did not consider the identity of the amino acid(s) of middle residue(s), only the structural digit(s). Q was partition function. The expression (in the logarithmic form) is illustrated in FIG. 22. The weights were then determined by weighted least square fitting to minimize the difference between the predicted populations and the actual populations observed in the MD simulations of the training sequences.

The dataset used included MD simulation results of a total of 581 sequences, where 495 sequences ran to 200 ns; 46 sequences were extended to 300 ns; 21 sequences were extended to 400 ns; 4 sequences were extended to 500 ns; 6 sequences were extended to 600 ns; 9 sequences were extended to 700 ns, among which 6 sequences were still being extended by even longer simulation time. Trajectories of the last 100 ns were used. NIP's (from 3D density profiles; comparing S1 vs. S2 (two different starting structures of the same cyclic peptide sequence, see Example section) were all above 0.9, except the 6 sequences which were still being extended. The 581 sequences were generated using a similar strategy as used by the semi-random training dataset for cyclic pentapeptides used before.

The test dataset used included a total of 50 random sequences, where 41 sequences ran to 200 ns; 8 sequences were extended to 300 ns; 1 sequence were extended to 600 ns. Trajectories of the last 100 ns were used. NIP's (from 3D density profiles; comparing S1 vs. S2) were all above 0.9.

The performance of linear StrEAMM (1,2)+(1,3)+(1,4)/random model were shown in FIG. 23. Generally, the fitted populations matched the observed populations for the training sequences well (FIG. 23 a). For test sequences, the Pearson correlation coefficient was 0.867 when comparing the predicted and the observed populations; the weighted error was 3.617 (FIG. 23 b).

### Neural Network StrEAMM Models for Cyclic Hexapeptides

Convolutional neural networks (CNNs) and graph neural networks (GNNs) were built using the same cyclic hexapeptide sequences as mentioned above. Cyclic peptide sequences are represented using a molecular fingerprint encoding scheme. Molecular fingerprints describe each amino acid's 2D structure as a set of substructures, which can then be represented as a 1 by 2048-bit vector containing 1s and 0 to denote the presence and absence of these substructures.

The CNN StrEAMM model's convolution layer is motivated by neighboring interactions. CNNs use convolutional layers to learn local interactions among the input features. This learning is achieved by applying filters (which perform the mathematical operation, the dot product) to a subset of features that are adjacent to each other. Our CNN models arrange the input representation of the cyclic hexapeptide sequence such that neighboring amino acids have their features adjacent in space (FIG. 24). Then, the CNN models use convolutional filters to encompass neighboring-like interactions (such as “(1, 2)” or “(1, 3)” interactions). The resulting vector of dot products is then the input layer into a standard multilayer perceptron, which is fully connected to a single hidden layer. After the input layer passes information to the hidden layer, the ReLU activation function is applied to enable non-linearity. Then, the hidden layer is fully connected to the output layer, which will predict the populations of 5,640 structures considered in the pool representing the structural ensemble. The softmax activation function is applied to the output layer to normalize the output to sum to 1.

### The Performance of the CNN StrEAMM Models on the Training and Test Sets for Cyclic Pentapeptides and Cyclic Hexapeptides.

For the cyclic hexapeptide dataset, the architecture with the lowest average mean squared error between the predicted populations and the populations observed in MD (after hyperparameter tuning and 3-fold cross validation) was the CNN (1, 2)+(1, 3)+(1, 4) StrEAMM model. For the 50 cyclic hexapeptide test sequences, the model has a weighted error (WE) of 2.55, weighted squared error (WSE) of 34.33, and Pearson R of 0.922 (FIG. 25). For the cyclic pentapeptide dataset, the architecture with the lowest average mean squared error between the predicted populations and the populations observed in MD (after hyperparameter tuning and 3-fold cross validation) was the CNN (1, 2) StrEAMM model. For the 50 cyclic pentapeptide test sequences, the model has a weighted error (WE) of 1.33, weighted squared error (WSE) of 6.11, and a Pearson R of 0.978 (FIG. 25).

### The GNN StrEAMM Models Create a Cyclic Peptide Graph Motivated by Amino Acid Neighbor Interactions

Beginning from the molecular fingerprint representation of a cyclic peptide (FIG. 24 a), the GNN StrEAMM model begins by reimagining the cyclic peptide as a graph. Each amino acid becomes one node of the graph, and edges of distinct types are added to the graph which connect the nodes and represent the (1, 2), (1, 3), and (1, 4) interactions in the peptide. To distinguish, for example, the peptides cyclo-(ARGVDE) (SEQ ID NO: 52) from cyclo-(EDVGRA) (SEQ ID NO: 82), forward and reverse interactions with respect to the peptide sequence are encoded with distinct edge types. As seen in FIG. 26, a cyclic pentapeptide has 4 different edge types representing forward (1, 2), reverse (1, 2), forward (1, 3) and reverse (1, 3) interactions; a cyclic hexapeptide has these four edge types in addition to forward (1, 4) and reverse (1, 4) edges for those additional distinct interactions.

The GNN StrEAMM Models Convert a Peptide Graph into a Structural Ensemble

Each length of peptide has a unique GNN StrEAMM model. The GNN takes a cyclic peptide graph, and first performs a graph convolution message passing step on the graph. This updates each node in the graph by considering each node's original fingerprint and the fingerprints of the other nodes connected by each edge type to the node. At this point, each node represents a combination of the initial fingerprint and information about the other amino acids in the cyclic peptide. Next, a ReLU activation function is applied, and the node representations are concatenated into a vector representation of length 5×2048 for a cyclic pentapeptide, or 6×2048 for a cyclic hexapeptide. This vector is transformed by a dense layer of 2048 nodes with the ReLU activation function into the structural ensemble for a cyclic peptide of the relevant length, normalized with the softmax activation function so that the values in the output structural ensemble sum to 1, or 100%. The ReLU activation function adds nonlinear operations to the model, helping the GNN to fit to nonlinear relationships.

The GNN StrEAMM model is trained for 1000 epochs using the Adam optimizer, shuffling data loaders in the case of FIG. 27, and non-shuffling data loaders in the case of FIG. 16 and FIG. 17, sum of squared errors loss function, and a batch size of 10 for the hexapeptides, 50 for the pentapeptides. For each peptide in the training datasets, the models are trained on the peptide itself, as well as cyclically permuted and enantiomer sequence inputs.

### The Performance of the GNN StrEAMM Models on the Training and Test Sets for Cyclic Pentapeptides and Cyclic Hexapeptides.

The GNN StrEAMM hexapeptide model on the 50 cyclic hexapeptide test sequences has a weighted error (WE) of 2.18, weighted squared error (WSE) of 22.15, and Pearson R of 0.945 (FIG. 27). The GNN StrEAMM pentapeptide model on the 50 cyclic pentapeptide test sequences has a weighted error (WE) of 1.32, weighted squared error (WSE) of 5.37, and a Pearson R of 0.976 (FIG. 27).

### StrEAMM can be Used to Provide Sequences Given a Target Structure

In addition to the development of our machine learning (ML) models for larger cyclic peptide sizes, the StrEAMM models can identify particular sequences that are predicted to have a high population of a desired structure. For example, our ML models can determine which cyclic pentapeptide sequences are predicted to have high populations of the structure ΛΛBλβ. To efficiently conduct a search of the sequence space and identify these optimal sequences, we have implemented a genetic algorithm, which is an optimization procedure based on the theory of evolution. Genetic algorithms start with a random subset of the sequence space, which we consider as the starting population. These sequences are evaluated based on their “fitness”, which in our case is their predicted population of some desired structure. Sequences that have a high predicted population of the desired structure (from the StrEAMM model) are selected to become “parents” and can pass on their sequence information to the next generation of sequences. Their “children” are generated by “crossover” events, which in our case would be the exchange of each parent's sequences at some cross-over point. Lastly, to achieve even better sampling, random mutations are allowed to occur with some probability in the new generation. With this new generation, the fitness evaluation, selection and crossover of parents, and random mutation events repeat in a cycle for a set number of generations (FIG. 28 a). The genetic algorithm we implemented to generate sequences that were predicted to have high populations of the structure ΛΛBλβ started with 1,000 randomly generated sequences, and the top 20% of the fittest individuals were selected to become parents. These parents then populated the new generation via a double crossover event (i.e., there were two randomly selected crossover points). We allowed this process to repeat for a number of generations and compared the sequences our genetic algorithm found to be the top 10 sequences with the “actual” top 10 sequences in the sequence space that have the highest population of the structure ΛΛBλβ. The “actual” results were determined by performing a complete search, which involved making predictions for all 15⁵=759,375 sequences and filtering the results for the sequences with high populations of structure ΛΛBλβ. After 5 generations, the genetic algorithm was able to successfully discover all the top 10 sequences (FIG. 28 b). The discovery of these optimal sequences using genetic algorithms is highly efficient, taking less than a second to generate.

### Property Prediction Enabled by Leveraging Structural Information

Structural information provided by StrEAMM can be leveraged to solve, for example, the challenges of optimizing both binding affinity and membrane permeability to develop membrane-permeable cyclic peptides for intracellular targets. It is difficult to train a ML model to predict the properties of cyclic peptides using only sequences and experimental data, because it is not possible for the model to decipher how sequence modifications impact the complicated conformational landscape of cyclic peptides, which in turn influences their properties. However, as our StrEAMM method enables us to efficiently predict cyclic peptide structural ensembles, one can leverage the structural information provided by StrEAMM and develop the first ML models that can accurately predict important drug-related properties of cyclic peptides.

As the Examples demonstrate, by considering the effects of both (1, 2) and (1, 3) interactions on a cyclic pentapeptide's structural preferences, we were able to use MD simulation results to train machine-learning models that are capable of quickly predicting MD-quality structural ensembles for cyclic pentapeptides in the whole sequence space. This approach greatly reduces the need to perform computationally expensive explicit-solvent simulations. Whether the predicted structural ensembles accurately match experimental results will depend on the force field used to generate the MD simulation results the model is trained on. The force field used here was the residue-specific force field 2 (RSFF2)^{36, 37} and TIP3P water model.³⁸ RSFF2 was previously shown to be able to recapitulate the crystal structures of 17 out of 20 cyclic peptides.³⁹ RSFF2 was also used to predict well-structured cyclic peptides, and the predicted results were supported by solution NMR experiments.^{24, 35} Should a different force field be preferred or an improved force field be developed, the approach reported here can be used to build new StrEAMM models for the chosen or improved force field by regenerating the MD simulation results and retraining the model.

The model can be extended to larger cyclic peptides, where it is possible that longer-range interactions beyond (1, 2) and (1, 3) pairs are also important. For example, cyclic hexapeptides tend to form a double-ended β hairpin, and in this case, we expect that the (1, 4) pair that forms intramolecular hydrogen bonds can be important at influencing the structural preferences. However, in the case of cyclic pentapeptides, the (1, 4) pair is equivalent to a (1, 3) pair and the (1, 5) pair is equivalent to a (1, 2) pair due to the cyclic nature of the molecule. Therefore, (1, 2) and (1, 3) interactions capture all the two-body interactions. Nonetheless, the current model performs nicely without including higher-body interactions, i.e. three-body interactions, four-body interactions, etc.

We observe that when the ring size increases, the number of interactions included in one simulation in the training set also increases. For example, a cyclic pentapeptide includes 5×(1, 2) interactions and 5×(1, 3) interactions, while a cyclic hexapeptide includes 6×(1, 2) interactions, 6×(1, 3) interactions and 6×(1, 4) interactions. Therefore, the number of compounds needed to observe all possible patterns of two-body interactions in a semi-random training set does not necessarily increase for cyclic peptides of larger sizes.

The Examples employ (1, 2) and (1, 3) interactions in the model for good interpretability. Neural networks may be used to train the model, which can be more difficult to interpret but may be able to embed complicated interaction patterns more easily.

The Examples include 15 D- and L-amino acids in the StrEAMM models. The models can be extended to have a larger size of amino-acid library (e.g., StrEAMM model (1,2)+(1,3)/sys37 extending to 37 amino acids using a systematic training dataset). To build StrEAMM model (1,2)+(1,3)/random37, one will need a larger number of training sequences than StrEAMM model (1,2)+(1,3)/sys37 in the training set when incorporating more types of amino acids. To be more efficient at incorporating various amino acids, instead of using one-hot encoding of the sequence, one can represent each amino acid using its chemicophysical properties or fingerprints to reduce the number of independent variables in the model. For example, after introducing the fingerprint encodings of amino acids, the StrEAMM model GNN/random was able to predict structural ensembles of cyclic peptides containing amino acids not present in the training dataset (FIG. 16), and achieve significant improvements by extending the training dataset with only a small amount of data (FIG. 17).

In our current map, the regions are well defined and fixed. In general, the binning map is capable of separating the major peaks of the Ramachandran plots of all amino acids in our analysis (FIG. 19). The model can also be extended to include beta amino acids, N-methylated amino acids, and nonpeptidic linkages etc. To describe the backbone of a beta-amino acid, one needs 3 dihedral angles, and a separate binning map is needed to describe the structure of beta-amino acids (it can be a 3D map, and not necessary a 2D map like the Ramachandran map we used in the paper). Similarly, one would need a separate binning map for nonpeptidic linkages. The structural digits for a cyclic peptide would be a mixing of digits from the Ramachandran map and the separate maps for those special amino acids and linkages.

The disclosed technology is capable of efficiently predicting complete MD-quality structural ensembles for cyclic peptides without direct MD simulations. The new models developed here can be used to quickly estimate structural descriptions of previously unsimulated cyclic peptides without the need to run any new MD simulations. For example, it takes <1 second to use StrEAMM Model (1,2)+(1,3)/sys or (1,2)+(1,3)/random to make a prediction of the structural ensemble for a cyclic pentapeptide, instead of days of running and analyzing an explicit-solvent MD simulation (approximately 80 hours using 15 Intel Xeon E5-2670 or 56 hours using 15 Intel Xeon Gold 6248+1 NVIDIA Tesla T4). After training, the model can predict structural ensembles for cyclic peptides of the same ring size in the whole sequence space. Such a capability of predicting structural ensembles of both well-structured and non-well-structured cyclic peptides should greatly enhance our ability to develop cyclic peptides with desired structures and even engineer their chameleonic properties.

### MD Simulations

The structural ensembles of cyclic peptides in water were sampled using bias-exchange metadynamics simulations^{32, 33} with the residue-specific force field 2 (RSFF2)^{36, 37} and TIP3P water model.³⁸

Two parallel bias-exchange metadynamics (BE-META) simulations starting from two different initial structures were performed for each cyclic peptide. The two initial structures were prepared using the UCSF Chimera package,¹ and the backbone RMSD between the two structures was ensured to be larger than 1.3 Å. The initial structure was solvated in a water box. The minimum distance between the atoms of the peptide and the walls of the box was 1.0 nm. Counter ions were added to neutralize the total charge of the system. Energy minimization was then performed on the solvated system using the steepest descent algorithm to remove bad contacts. The solvated system underwent two stages of equilibrations. In the first stage, the solvent molecules were equilibrated while restraining the heavy atoms of the cyclic peptide using a harmonic potential with a force constant of 1,000 kJ·mol⁻¹·nm⁻². This stage of equilibration consisted of a 50-ps simulation at 300 K in an NVT ensemble and a following 50-ps simulation at 300 K and 1 bar in an NPT ensemble. The second stage of equilibration was performed without restraints and consisted of a 100-ps simulation at 300 K in an NVT ensemble, followed by a 100-ps simulation at 300 K and 1 bar in an NPT ensemble. The production simulations were performed at 300 K and 1 bar in an NPT ensemble. The equations of motion were integrated by the leapfrog algorithm with a time step of 2 fs. Bonds involving hydrogen were constrained with the LINCS algorithm. Electrostatic interactions, van der Waals interactions, and neighbor searching were truncated at 1.0 nm. Long-range electrostatics were treated using the particle mesh Ewald method with a Fourier grid spacing of 0.12 nm and an order of 4. A long-range dispersion correction for energy and pressure was applied to account for the 1.0 nm cut-off of the Lennard-Jones interactions. Five extra improper dihedrals related to the H, N, C, O atoms of the peptide bonds were applied to suppress the formation of cis bonds. It was ensured the data used in the analysis were free of cis peptide bonds.

BE-META simulations were performed using GROMACS 2018.62 patched by PLUMED 2.5.1 plugin.³ In each BE-META simulation, there were 10 biased replicas, with five biasing the 2D collective variables (ϕᵢ, ψᵢ) and five biasing the 2D collective variables (ψᵢ, ϕᵢ₊₁). These collective variables were chosen according to the observation that cyclic peptides usually switch conformations through coupled changes of two dihedrals involving (ϕᵢ, ψᵢ) or (ψᵢ, ϕᵢ₊₁).⁴ In addition, five neutral replicas (i.e., replicas with no bias) were used to obtain the unbiased structural ensemble for later analysis. Dihedral principal component analysis was used to analyze the trajectories. Normalized integrated product (NIP)⁵ between the two parallel simulations of each cyclic peptide was calculated in the 3D space spanned by the top three principal components to monitor the convergence of the simulations. The lengths of the BE-META simulations were 100 ns for most of the cyclic peptides and were extended for some peptides until the NIPs were larger than 0.9 (an NIP value of 1.0 would suggest perfect similarity). Trajectories in the last 50 ns of the neutral replicas of both parallel simulations were combined for each cyclic peptide and used for further structural analysis.

### Structural Analysis

Conformations of cyclic pentapeptides were described by the backbone dihedrals {ϕᵢ, ψᵢ; i=1-5}. We found that the structure of a B turn plus a tight turn (α_{L}, α_{R}, γ, or γ′ turn) used by Slough et al.²⁴ could not describe all possible structures, so we used another method by discretizing the (ϕ, ψ) space into different regions and denoting each region with a structural digit. To do this, we first analyzed the (ϕ, ψ) space of cyclo-(GGGGG) (SEQ ID NO: 83). Because Gly is achiral and the most flexible amino acid, it is assumed to provide a universal binning map that can be used by others, including both D- and L-amino acids. The (ϕ, ψ) distribution of cyclo-(GGGGG) (SEQ ID NO: 83) was first clustered by a grid-based and density peak-based method with centroids identified.⁴³ All the grid points in the Ramachandran plot were then assigned to their closest centroid, forming 10 regions, each of which was assigned a letter: Λ, λ, Γ, γ, B, β, Π, π, Z, or ζ (FIG. 6). As expected, the map is centrosymmetric. With this map, each conformation of a cyclic pentapeptide can be represented by a five-digit string. For example, the conformation “Πλζλβ” indicates that the first residue of the cyclic pentapeptide is in the “Π” region of the Ramachandran plot, while the second, third, fourth, and fifth residue fall in the “λ”, “ζ”, “λ” and “β” regions, respectively.

### Datasets

We used data from the MD simulations to train and test the models, because experimental information of structural ensembles of cyclic peptides is scarce and difficult to obtain. Fifteen amino acids were used in this study: G, A, V, F, N, S, D, R, a, v, f, n, s, d, and r; lowercase letters denote D-amino acids. These amino acids were chosen to include Gly (achiral), and both the L- and D-form of alanine (a vanilla amino acid), valine (with β branching), phenylalanine (with an aromatic side chain), asparagine (with an amide group in the side chain), serine (with a hydroxyl group in the side chain), aspartate (with a negatively charged side chain), and arginine (with a positively charged side chain).

Training dataset for Scoring Functions 1.0 and StrEAMM Model (1,2)/sys (Dataset 1).

This dataset included 106 systematic sequences: Cyclo-(GGGGG) (SEQ ID NO: 83), cyclo-(X₁GGGG), cyclo-(X₁X₂GGG), and cyclo-(X₁X₂GGG, with Xᵢ being one of the seven L-amino acids and xᵢ being one of the seven D-amino acids. Generally, each sequence contained one unique nearest-neighbor pair with the rest of the sequence filled by Gly's. Gly was used as the filler amino acid because it is achiral and has no sidechains, allowing sampling the most conformational space. The enantiomers of these cyclic peptides, i.e., cyclo-(x₁GGGG), cyclo-(x₁x₂GGG), and cyclo-(x₁X₂GGG) were not simulated, and their structural ensembles were inferred from the 105 simulated cyclic peptides.

### Training Dataset for StrEAMM Model (1,2)+(1,3)/Sys (Dataset 2).

This dataset included 204 systematic sequences: Cyclo-(GGGGG) (SEQ ID NO: 83), cyclo-(X₁ GGGG), cyclo-(X₁X₂GGG), cyclo-(X₁X₂GGG), cyclo-(X₁GX₂GG), and cyclo-(X₁Gx₂GG), with Xᵢ being one of the seven L-amino acids and xᵢ being one of the seven D-amino acids. Each sequence contained one unique nearest-neighbor or next-nearest-neighbor pair with the rest of the sequence filled by Gly's. Again, the enantiomers of these cyclic peptides were not simulated, and their structural ensembles were inferred from the 203 simulated cyclic peptides.

### Training Dataset for StrEAMM Model (1,2)+(1,3)/Random (Dataset 3):

This dataset included 705 “random” sequences that were generated using the following protocol. When building the sequence pool, we required (1) the number of sequences to be as small as possible, (2) X₁₋X₂ to sandwich all the possible amino acids, i.e., all X₁X₂X₃ patterns were observed, (3) no enantiomers and (4) not double-counting sequences that were the same cyclic peptides after cyclic permutation.

### Test Dataset (Dataset 4):

50 random sequences were used as the test dataset. It was ensured that there were no equivalent sequences after cyclic permutation and there were no two sequences that were enantiomers to each other.

### Lists

List 1. The structure pool used in the analysis.
The pool includes 550 structures (275 enantiomer pairs) whose populations
(either one structure or its enantiomer, or both) were larger than 0.1%
(500 frames) in at least one of the cyclic peptides in Datasets 1-3.

λΓλλζ ΛγΛΛZ λπΓλλ ΛΠγΛΛ ΓγBλZ γΓβΛζ γZΛΠΠ Γζλππ γBζπB ΓβZΠβ γZΠγΠ
ΓζπΓπ λλβΓπ ΛΛBγΠ Πλλζβ πΛΛZB λΓλΛΓ ΛγΛλγ βΠβΓζ BπBγZ ΛΓλΛΛ λγΛλλ
ΛΓζλλ λγZΛΛ BβΓγΛ βBγΓλ BβΠλγ βBπΛΓ ΠλBπZ πΛβΠζ ΠπBβZ πΠβBζ πΛBγZ
ΠΛβΓζ πΠζπB ΠπZΠβ πBζλζ ΠβZΛZ ΛΠγBβ λπΓβB ΠλZΛB πΛζλβ βΓγΛλ BγΓλΛ
πΛBβB ΠλβBβ πBβΠζ ΠβBπZ ZΓγΠπ ζγΓπΠ ζBΓγΓ ZBγΓγ πΓBπB ΠγβΠβ λλλΓζ
ΛΛΛγZ πΛΛβΓ ΠλλBγ λΓζγΛ ΛγZΓλ λλβΛΠ ΛΛBλπ ΛΓZΓβ λγζγB BζπΛΛ βZΠλλ
βΛγΓζ BλΓγZ βBλλΓ BβΛΛγ ζλβΛζ ZΛBλZ λλBζβ λλβZB γγBλζ ΓΓβΛZ ζλγΛλ
ZΛΓλΛ ZΓγΓβ ζγΓγB γγBπΠ ΓΓβΠπ Γλλζβ γΛΛZB ΓγZΠβ γΓζπB ZΓλΓΛ ζγΛγΛ
λΛBγΛ ΛλβΓλ ΓγΓβπ γΓγBΠ ΠλZΠβ πΛζπB ΠγΓγΛ πΓγΓλ Bγζλβ βΓZΛB ΠλβΠζ
πΛBπZ ΓλZΓβ γΛζγB βΠπΓζ BπΠγZ ζγΓγΠ ZΓγΓπ BλζλZ βΛZΛζ ΠλζγB πΛZΓβ
ZΓγΠγ ζγΓπΓ λβΛΛΛ ΛBλΛΛ πΓβΓβ ΠγBγB λγΛγΠ ΛΓλΓπ ΠπZBπ πΠζβΠ γΛβΠλ
ΓλBπΛ ΛΛZΛβ λλζλB ζλλΛλ ZΛΛλΛ ζλZΛB ZΛζλβ ΛΓγZΠ ΛγΓζπ βΠΛγΠ BπΛΓ
λλπΓζ ΛΛΠγZ ζγBπB ZΓβΠβ γΛγZΓ ΓλΓζγ ΠζζΛβ πZZΛB ΓπΠλπ ΥΠπΛΠ γBλΓλ
ΓβΛγΛ ΓγΓππ γΓγΠΠ λγζΠζ ΛΓζπZ ΛΠβΛZ λπBλζ ΓβZΛΛ γBζλλ λΛΓγΛ ΛλγΓλ
γζλλλ ΓZΛΛΛ λλΛγB ΛΛλΓβ ΓβBγZ γBβΓζ λλζβB ΛΛZBβ λΓλΓλ ΛγΛγΛ ZΠπΓγ
ζπΠγΓ ΓπΓγΛ γΠγΓΛ ZΛΓγZ ζΛγΓζ Γλλζπ γΛΛZΠ πΛβΓζ ΠλBγZ γΓβΓΓ ΓγBγγ
ΛΛΛΠπ λλλπΠ ΓλζγZ γΛZΓζ ΠγζπZ πΓZΠζ γBζλζ ΓβZΛZ λγΓγΛ ΛΓγΓλ λλζγΠ
ΛΛZΓπ ΓλZΓγ γΛζγΓ πΠζλζ ΠπZΛZ γΛΛζΛ ΓλλZΛ ΛπΠπΓ ΛΠπΠγ ZΓΓγΛ ζγγΓΛ
ΛζβΛζ ΛZBλZ ΛΓζλZ λγZΛζ πΠπBζ ΠπΠβZ ΛΛγBπ λλΓβΠ πBβΓζ ΠβBγZ λγBλB
ΛΓβΛβ λλBπB ΛΛβΠβ λππΛB ΛΠΠλβ πΠγBζ ΠπΓβZ γΛBπB ΓλβΠβ γΓZΛB Γγζλβ
BγBπZ βΓβΠζ πΛζπΠ ΠλZΠπ ΛΛBπZ λλβΠζ λΓβΛB ΛγBλβ πΠβΠζ ΠπBπZ ΠλβΛβ
πΛBλB ZΛBγζ ζλβΓζ γΛζλζ ΓλZΛZ ΠγβΓπ πΓBγΠ λλλλB ΛΛΛΛβ λγBγζ ΛΓβΓZ
ΠγZΓπ πΓζγΠ λπΓβΓ ΛΠγBγ ΠπZΓβ πΠζγB ΓγΛΛγ γΓλλΓ πΓζπB ΠγZΠβ ζπΓγΓ
ZΠγΓγ ΓβZΓβ γBζγB λπΛΓζ ΛΠλγζ ΠζβΛZ πZBλζ ΠλλBπ πΛΛβΠ λγBγΠ ΛΓβΓπ
λπΓλζ ΛΠγΛZ ΓζγΛγ γZΓλΓ ΓλζπΓ γΛZΠγ ΛBζλγ λβZΛΓ γΓβΓζ ΓγBγZ ΠγΛBγ
πΓλβΓ ΛΠγΓβ ΛπΓγB γΠλλλ ΓπΛΛΛ γZBλζ ΓζβΛZ ΓβΠγΛ γBπΓλ ZΓλλγ ζγΛΛΓ
γΠβΛB ΓπBλβ ΠγζγZ πΓZΓζ λζζγB ΛZZΓβ λβΛΛΠ ΛBλλπ ΠγβΛZ πΓBλζ BλλΓπ
βΛΛγΠ γZΠλλ ΓζπΛΛ βΠπΠγ BπΠπΓ γZBλλ ΓζβΛΛ ΛBζγZ λβZΓζ ΛΛZΓZ λλζγζ
πΓλγΠ ΠγΛΓπ βΓβΓζ BγBγZ ΛZZΛZ λζζλζ λγζλζ ΛΓZΛZ πΓZΛΠ ΠγζΛπ λλλΓλ
ΛΛΛγΛ γΛBλΛ ΓλβΛλ λβΠγΠ ΛBπΓπ ΓλβΠγ γΛBπΓ ΛΓλζβ λγΛZB πΠπΠζ ΠπΠπZ
ΓγΛΛZ γΓλλζ ΓγZΓβ γΓζγB ΠγZΛB πΓζΛβ ΓλγΛλ γΛΓλΛ ΛΓλΓβ λγΛγB BβΛΛΛ
βBλλλ πΠβΓζ ΠπBγZ βλΓγB BλγΓβ ΛΓλζγ λγΛZΓ γΛβΛB ΓλBλβ ΛZBγZ λζβΓζ
ΓγΠγΛ γΓπΓλ λλZΓζ ΛΛζγZ ΠλZΓβ πΛζγB βΠλπΓ BπΛΠγ πΠλλζ ΠπΛΛZ γΠπΠγ
ΓπΠπΓ ΠλζπZ πΛZΠζ πΓλβΠ ΠγΛBπ βBπΠλ BβΠπΛ BπΛΛΛ βΠλλλ ΓγΓγZ γΓγΓζ
λγΓπB ΛΓγΠβ λβBπB ΛBβΠβ πΛβΛB ΠλBλβ ΛΓγΛβ ΛγΓλB ΓβΓγΛ γBγΓλ γΛBλΓ
ΓλβΛγ ΠγβΓβ πΓBγB πΠπΓζ ΠπΠγZ ΠγZΓβ πΓζγB πΓβΠζ ΠγBπZ ΓγZΛZ γΓζλζ
ΓλZΛΛ γΛζλλ πΠπΠλ ΠπΠπΛ λλζπB ΛΛZΠβ γΓβΓλ ΓγBγΛ πΠπBλ ΠπΠβΛ γΠπBλ
ΓπΠβΛ BγΛγΛ βΓλΓλ λγZΓζ ΛΓζγZ ΛZΛΓπ λζλγΠ λλλλλ ΛΛΛΛΛ λζπΛΓ ΛZΠλγ
ΠγZΛZ πΓζλζ Γλγλγ γΛΓλΓ πΛBλβ ΠλβΛB ΓπΛBγ γΠλβΓ βΓλλλ BγΛΛΛ ΓγΛγΛ
γΓλΓλ γΛΓζλ ΓλγZΛ ΓγΠπΓ γΓπΠγ λλβΛζ ΛΛBλZ ΓγBπΓ γΓβΠγ λλγΛΓ ΛΛΓλγ
ΠλζγZ πΛZΓζ ΛΛγΓπ λλΓγΠ ΛΓβΠβ λγBπB πΛΛγΠ ΠλλΓπ ΠβBλβ πBβΛB ΠλZΛZ
πΛζλζ ΓβΠπΛ γBπΠλ πΓβΓζ ΠγBγZ γΛBλζ ΓλβΛZ ΛΛBΛζ ΛΛβΛZ BλγΛΛ ΛΛΓλλ
ΠλβΠβ πΛBπB ζλλγΓ ZΛΛΓγ λβBγB ΛBβΓβ ΓλβΓβ γΛBγB ΛΛβΓβ λλBγB ΛBβΛZ
λβBλζ πΓβΛΠ ΠγBλπ ΛBγΓγ λβΓγΓ ΛΛZΛΛ λλζλλ πΓβΛΓ ΠγBλγ ΠπΛΓπ πΠλγΠ
γBβΛB ΓβBλβ ΓπΛΓγ γΠλγΓ λλλγB ΛΛΛΓβ λβZλβ ΛBζλβ Γλζλβ γΛZΛB λλλγΓ
ΛΛΛΓγ λλγΛB ΛΛΓλβ ΠπΛBγ πΠλβΓ ΛΛζλβ λλZΛB ΓγΓγΛ γΓγΓλ Γλζλλ γΛZΛΛ
λλβΓζ ΛΛBγZ Πγζλβ πΓZΛB λζλπΠ ΛZΛΠπ λλζγB ΛΛZΓβ ΛΓγBπ λγΓβΠ ΛΛZΛZ
λλζλζ BλBΛZ βΛBλζ γΓβΛB ΓγBλβ βΛΛγΓ BλλΓγ ΓγΛZΛ γΓλζλ ΠπΛΓγ πΠλγΓ
ΠπBλβ πΠβΛB ΠλβΠπ πΛBπΠ ΛΓβΓβ λγBγB λγBλζ ΛΓβΛZ ΠλβΛZ πΛBλζ ΓβΛΓγ
γBλγΓ ΛΓζλβ ΛγZΛB πΛBγB ΠλβΓβ λλβΛB ΛΛBλβ πΓβΛB ΠγBλβ Πλζλβ πΛZΛB

List 2.
50 random cyclic peptide sequences in the test dataset (Dataset 4).

| Sequence: | SEQ ID NO: | Sequence: | SEQ ID NO: | Sequence: | SEQ ID NO: |
|---|---|---|---|---|---|
| rNDsF | | ANDnA | | SSrsA | |
| nsaaF | | SdFrS | | RsFDS | |
| AsNAr | | FAdNA | | FvrFR | |
| VssSN | | DfaNv | | VSadn | |
| FdNfA | | AsNVs | | vsVAG | |
| vrdvA | | DdFVs | | nvDnF | |
| DAvsD | | avVrr | | arFGa | |
| fNvAA | | SrGnR | | vfDsR | |
| RaRDR | | NNDas | | dfRNd | |
| VaAVn | | dNsGV | | FaanA | |
| RfdNr | | RAvRs | | | |
| snGAa | | dVsrf | | | |
| RADaS | | nrRAv | | | |
| nARRD | | DdSrD | | | |
| sDAsF | | GNsrn | | | |
| SVFdR | | fffaa | | | |
| avNSd | | NAnNS | | | |
| RDAvs | | SaARF | | | |
| FsGna | | fnDSd | | | |
| SVFAa | | sdSfd | | | |

List 3.
705 semi-random cyclic peptide sequences in the training dataset (Dataset 3) for
StrEAMM Model (1,2) + (1,3)/random. (SEQ ID NO: 93, 156, 167, 168, 226, 227, 284, 302,
342, 367, 368, 398, 438, 503, 548, 561, 564, 583, 585, 648, 691, 693, 720, 755, 790)

| | SEQ ID | | SEQ ID | | SEQ ID | | SEQ ID |
|---|---|---|---|---|---|---|---|
| Sequence: | NO: | Sequence: | NO: | Sequence: | NO: | Sequence: | NO: |
| SnSVa | | nddRr | | ADsdn | | rnSfd | |
| dDdfS | | DaaVv | | SAsAf | | GdsGn | |
| SNNGR | 93 | fffsN | | vVdDG | | FaGFD | |
| GrNDa | | dfRFv | | NfaAF | | vsSGS | |
| FvrAS | | aRVsN | | FffGR | | DDGFs | |
| ndaVV | | RAFfn | | DaFDs | | AAFsG | |
| fVsaf | | DnFSA | | ASSAG | | nANDG | |
| ARDan | | Nsndr | | rrdaS | | NGVGa | |
| nVASD | | rGDav | | vvaSd | | rFfFS | |
| fddsv | | aDAAV | | Nvfrd | | anrRn | |
| dDVrF | | DVRRV | 156 | vsrAN | | fSNDR | |
| aAfvs | | VNaDS | | NGvnr | | RdrAV | |
| VrrAR | | dFvRA | | arrnG | | NFAVA | 226 |
| vSAfs | | RdSNF | | DrGRa | | SVFVR | 227 |
| NdnRG | | GsnRv | | AvFVS | | VrRAV | |
| srnaf | | nvNDG | | GRRvs | | FRnvn | |
| SDRan | | nNnGA | | VnvFS | | SfGFR | |
| dAVSD | | DrDFn | | fDNsd | | NRDRd | |
| Nrrsr | | nffds | | nsfva | | DraAr | |
| dGSNd | | dRfrA | | nrsAN | | NfNdv | |
| GVvFv | | aVRvn | | vNNFN | | rRraN | |
| FfNsN | | NDAGG | 167 | fRRSR | | faarn | |
| dFrNf | | FGSAF | 168 | DdnfD | | DsFRv | |
| SsVGd | | NDDsR | | dGVaV | | VNrSS | |
| dGfFa | | rAAGV | | aFfSf | | frvdd | |
| rSRSd | | FFFSf | | DaRfF | | adnva | |
| ASrdf | | DFsaV | | nSGdD | | frrSs | |
| ndvaR | | AsGSf | | DdFaA | | RvvdG | |
| rRFSS | | svaFN | | RnsnD | | aaanF | |
| nadda | | rNanv | | vnRDF | | raSAG | |
| RNvvN | | vrSDN | | NDVAs | | vSdnF | |
| AVFsv | | nvSvF | | Rsann | | SVAaf | |
| SaGAa | | VGDrd | | fvrrv | | RfANV | |
| dSFnS | | fFAva | | sfVVf | | VRnrf | |
| aDadf | | aFFds | | vGaSV | | NVnVa | |
| dVDAa | | rAarS | | nAdvA | | GdrRD | |
| afAnS | | vnSvd | | DnaNn | | FavVA | |
| SGvRR | | RNnFD | | ArADF | | AaaDS | |
| nVvVF | | dDSdN | | DGrvA | | RvARn | |
| DfNfs | | aAdar | | AFvNn | | VNnVD | |
| FSDrS | | VSndn | | SdVss | | sfNar | |
| DrnDf | | vaVfN | | SFfAR | | RDsGV | |
| AvDAR | | nDGSS | | rRNrF | | vNSAA | |
| GnNDN | | fdGdA | | vVrvs | | Nrdnn | |
| RRDAn | | RaFan | | dNfrf | | FDdDv | |
| VVAnD | | ANRsN | | ssrvN | | rVFaV | |
| fVFRD | | Fnfrs | | GGvNr | | vfsSf | |
| VSRvf | | SDRvG | | FGasr | | aNFRS | |
| fdVNR | | FsVDD | | GnsVF | | DNVAD | 367 |
| ARRNd | | NVfrn | | FDGfs | | AADAN | 368 |
| rRdsA | | nNdsD | | vssfD | | Fdasf | |
| vVaGn | | rAGSG | | RsDSv | | fFrnR | |
| dssVS | | VDNrA | | VGAdN | | Rvdnd | |
| GNVan | | RNANS | 302 | DSSGn | | SFAAR | |
| FrfSD | | sNVSf | | dfGAN | | RfaNn | |
| fnaGv | | fFfDV | | aRFnr | | GrfAn | |
| NRSvf | | RsRAf | | FNAdV | | FaFrG | |
| Vffaf | | avvsf | | fsnVF | | sSsvF | |
| Fssns | | rSasV | | GFFAF | 342 | vGGna | |
| rDNFr | | GfRnn | | aSSNS | | RnASG | |
| DdGAG | | asGrr | | RrfaR | | dFsNN | |
| AaFnF | | vrfGv | | rDdvd | | ASdRR | |
| rAsFa | | FRRnS | | vAaNa | | VRFdV | |
| NndDr | | sVVDd | | RvSaA | | assNn | |
| rDaAv | | AAANa | | fNDrR | | SGGrD | |
| sNSNr | | RAnvd | | RrAvV | | vvvfA | |
| VNsra | | VnDNa | | SfnfR | | svNVD | |
| frGvS | | nGvAF | | GfSRA | | GNrfA | |
| SSANN | | sFvfG | | FRdVn | | NdVva | |
| aGssG | | GsNRF | | Snvff | | SaVFd | |
| SFGGF | 284 | fGNGA | | AdSSd | | dSfsa | |
| VdAnG | | dDRsr | | snrvS | | Fdfdv | |
| NvDVv | | aAVGv | | GDvNd | | DrsSR | |
| rNAfN | | vAArn | | fVrGV | | AvGdV | |
| vRvra | | SVvsN | | RFRfD | | VvvRN | |
| rdRVv | | GfNGr | | VsVRA | | SGVSf | |
| dFfdR | | GSGsv | | ndGnF | | SASaR | |
| naFvs | | srSdG | | vDdRa | | dndSV | |
| RsfRa | | Vfnvv | | NAaVS | | SRnDd | |
| VGRVS | 398 | fRDNN | | SSFsA | | GFNVV | 503 |
| DfFRF | | arDrV | | NGdFd | | Fsrff | |
| SSRVf | | RGaAG | | rFasa | | ARrRG | |
| ddVfR | | VFSnF | | nnsGF | | sdAvd | |
| adASR | | Afasn | | DnrAF | | GanNa | |
| dNSGa | | FVGNA | 438 | ADvns | | vFASN | |
| ardFS | | VSFAf | | DfndA | | AdDFV | |
| VSSFr | | Arvna | | RaaGN | | FDSNs | |
| sSrnV | | aAsfS | | GnnaR | | VsGfv | |
| NAASv | | NDsas | | SnRNG | | vVnaN | |
| rnndN | | AFSRv | | fvvnV | | frSAS | |
| DFdvf | | RsvRV | | RNNDf | | DDASa | |
| vaGRD | | nFdad | | raDVF | | SavFf | |
| afvDG | | aAaRd | | VAdsr | | AvAsD | |
| FSNGa | | drrFV | | AFVrd | | DRRaG | |
| daava | | VddNA | | anfdA | | GDDfv | |
| Annfn | | sNvnd | | rvvGv | | SfVND | |
| SRFAs | | DaSff | | aFdra | | rAnFF | |
| RrDVD | | vn Dss | | SsavS | | sFdns | |
| VvAfA | | GsaDG | | sRrGr | | dSArd | |
| sVnAv | | ASSfN | | asvrR | | vnnFf | |
| FfrRs | | RRRGr | | SGNvV | | sssdR | |
| SRvVG | | VVVFv | | aFRsS | | GGGaV | |
| nFNFs | | SVfGr | | vdVda | | nGFdR | |
| dFnsv | | VnsRD | | NafaS | | GNSsr | |
| asDGN | | fDGVA | | DaNSV | | FrdGv | |
| nNVFF | | nAGaD | | vVsdd | | NvdAr | |
| vDnnD | | dnGnR | | NfGaa | | DNdNs | |
| SNRVn | | aDFDV | | VrNNr | | ArarG | |
| drVfD | | rRSns | | nfsGN | | RsnNS | |
| FGRdG | | SRRFV | | DRGFr | | SssaG | |
| sasFr | | GVVsf | | adsDd | | dGGSF | |
| NDdNa | | nvnAs | | frDfA | | NVNvS | |
| VrDsA | | DNDnG | | GRrSv | | rfrNd | |
| fvAGn | | nsNrD | | FVvdN | | NSfRr | |
| SnaSv | | dafGG | | nNAnV | | DAFdd | |
| fdDaD | | aSRFF | | FDrAr | | Ansaa | |
| RaASA | | vsRsd | | sAvnv | | VSsSD | |
| NNSda | | SDvVD | | ffnDA | | nrVGr | |
| rNRva | | dANfA | | RfsVA | | DvfFv | |
| vrVNS | | Vavnf | | VRGfn | | GAVNF | 648 |
| rFNDv | | DSnGR | | RafrV | | RNfDS | |
| sAAdf | | ARNFn | | SvvDF | | NvGAD | |
| dNNAs | | dnDaG | | DffvS | | rfRvD | |
| nNsfd | | GdfvF | | NfSFD | | sSVrG | |
| FVVNG | 548 | NADRV | 583 | aGGfd | | VadrG | |
| sffrF | | DnfSn | | SFFRa | | FGDVr | |
| RArGa | | VVSFV | 585 | svRdA | | dRFsS | |
| GvDSR | | vVvSr | | dVrns | | VGVnR | |
| DvGnr | | rssAd | | rVdNR | | RSSvD | |
| ANFDv | | AGsVN | | vfarV | | vdSGf | |
| RVVdV | | nFGfr | | sGGDG | | NadFR | |
| AaDRA | | RvNaa | | vArsv | | sVfAA | |
| nSAVn | | SaDNS | | NnvGN | | nArfn | |
| SsRdR | | DnvRD | | nSsDs | | rNVsr | |
| ASVRs | | VNNaF | | vnGsS | | GffAa | |
| ndfVG | | SSSDs | | NDFGd | | nnnrS | |
| AasSn | | dddAG | | vASnN | | DDDvd | |
| VDGDA | 561 | NdaDs | | Grdrv | | fDArS | |
| fAFAn | | dfnSR | | FvavN | | DFNns | |
| rAfnG | | aRDdV | | RvFNd | | AsrDG | |
| VGSDD | 564 | fVDvR | | Gdnrr | | FGrVs | |
| fFdAA | | rfDfS | | FaDvv | | RFrFn | |
| GRffR | | vRARd | | dsFFs | | VsvsD | |
| NFSdf | | sSNfn | | fanDF | | SnfFN | |
| rsdaR | | nSSAd | | RSDAS | | NnnvD | |
| ADSrD | | Dddna | | vrnRr | | rDDNA | |
| FRrFv | | fGfVn | | asRaV | | FdFGN | |
| dsRnV | | VAGFf | | DSsNG | | GvdaF | |
| aGVDf | | fnFsR | | AdGrF | | FNaSs | |
| NfvNG | | rvaDD | | fFVfd | | RVArr | |
| arRvR | | sAaAN | | ARsVs | | SvAvf | |
| SvVNV | | GnSnr | | nVGfG | | dNnNR | |
| FFNrv | | NRGdR | | aafRV | | fsdDs | |
| sFSGv | | SrsVd | | SandV | | nRSGD | |
| GsdFN | | dvGfa | | dSDaf | | DVdFA | |
| SAFRG | | SFaRN | | Svasd | | NAVSF | |
| nVVra | | DFRVF | 720 | NGGRA | 755 | FFDDS | 790 |
| rFFVn | | saSGR | | GdvDf | | SAnds | |
| fFnar | | DaVsv | | FaNAR | | GfAra | |
| AddFD | | dvrsD | | vDDar | | SrNFF | |
| naasA | | FNRSr | | AsaNR | | GSdFD | |
| SVsDv | | fVdvv | | nGSrV | | nAVAA | |
| DGRNS | 691 | GdGNs | | rdsfn | | | |
| RGDfs | | snnVr | | sdrFS | | | |
| VFARG | 693 | VDndF | | Gavsd | | | |
| Sddrd | | fADas | | nDDRD | | | |
| VvRSV | | SGFvv | | GVsnG | | | |
| fNVdf | | NvRFN | | FfGDF | | | |
| NnSrS | | RfGSR | | fNnRn | | | |
| VNdSn | | dFFvA | | GfDnN | | | |
| fradD | | NNNRn | | FRADr | | | |
| VvnNf | | rrrVD | | GVNfF | | | |
| dRDRV | | FrvRn | | DsrsG | | | |
| aVAVf | | rafNS | | AGvGF | | | |
| sVafD | | vSRrd | | SGAFr | | | |
| dDNRR | | adRGS | | Drfss | | | |
| aArVV | | saRaN | | AvRGG | | | |
| DSaaS | | GSVSr | | rnAAn | | | |
| FANra | | nNFaf | | avfRA | | | |
| RfvFf | | fNNVr | | sFVaF | | | |
| VRNR | | Rrrfv | | GsfSs | | | |

List 4.
75 random cyclic peptide sequences in the test dataset (Dataset 6) for StrEAMM model
(1,2) + (1,3)/sys37, including 37 types of amino acids. SEQ ID NOs: 812 and 847.

| Sequence: | SEQ ID NO: | Sequence: | SEQ ID NO: | Sequence: | SEQ ID NO: |
|---|---|---|---|---|---|
| AaGGr | | AqWLw | | NHyFI | |
| EekVh | | fLWSI | | rddwn | |
| GYEtS | | histl | | TddDk | |
| kKEct | | IWrHh | | WtYdE | |
| qaqQH | | qTySW | | dyqDk | |
| RQwsv | | sSnvf | | GyATS | |
| tlqIL | | vQNGk | | lyyKA | |
| YdFAm | | YwnlK | | nSLtc | |
| AewYN | | ASFsY | | rICAm | |
| emfAR | | FrMRM | | Teslq | |
| HDckk | | HSfCT | | YciGr | |
| kqRef | | MsMTK | | | |
| qcyAf | | qwHca | | | |
| rtiGN | | SvVid | | | |
| tlWci | | VTrDI | | | |
| ynWvy | | caTNv | | | |
| AHCVE | 812 | FVnTK | | | |
| FdFKF | | hwtiD | | | |
| HDIkt | | NhfwK | | | |
| LIHHK | | QWRNH | 847 | | |
| qlqrV | | Syhww | | | |
| SdNTI | | WFFqN | | | |
| TqGdv | | DeSMA | | | |
| ySmmw | | GcEQd | | | |
| ANIRQ | | IEKst | | | |
| FlcsH | | nhSYY | | | |
| hIMKy | | raNaK | | | |
| LMdCW | | taEVR | | | |
| QISFV | | wHseh | | | |
| sDyeT | | dhKYA | | | |
| vccVe | | GqqDE | | | |
| YvrNC | | INfWm | | | |

List 5.
The Dataset 6 in List S4 was divided into two sub datasets, Dataset 6.1 and Dataset 6.2.
Dataset 6.1 was used for training the StrEAMM GNN/random37 model; Dataset 6.2 was used for
testing both the StrEAMM GNN/random model and the StrEAMM GNN/random37 model.
Dataset 6.1: SEQ ID NOs: 881 and 905.

| Sequence: | SEQ ID NO: | Sequence: | SEQ ID NO: | Sequence: | SEQ ID NO: |
|---|---|---|---|---|---|
| AaGGr | | AqWLw | | dhKYA | |
| EekVh | | fLWSI | | GqqDE | |
| GYEtS | | hlstl | | INfWm | |
| kKEct | | IWrHh | | NHyFI | |
| qaqQH | | qTySW | | rddwn | |
| AewYN | | ASFsY | | dyqDk | |
| emfAR | | FrMRM | | GyATS | |
| HDckk | | HSfCT | | lyyKA | |
| kqRef | | MsMTK | | nSLtc | |
| qcyAf | | qwHca | | rICAm | |
| AHCVE | 881 | caTNv | | | |
| FdFKF | | FVnTK | | | |
| HDIkt | | hwtiD | | | |
| LIHHK | | NhfwK | | | |
| qlqrV | | QWRNH | 905 | | |
| ANIRQ | | DeSMA | | | |
| FlcsH | | GcEQd | | | |
| hIMKy | | IEKst | | | |
| LMdCW | | nhSYY | | | |
| QISFV | | raNaK | | | |
| RQwsv | | TqGdv | | YwnlK | |
| tlqIL | | ySmmw | | SvVid | |
| YdFAm | | sDyeT | | VTrDI | |
| rtiGN | | vccVe | | Syhww | |
| tlWci | | YvrNC | | WFFqN | |
| ynWvy | | sSnvf | | taEVR | |
| SdNTI | | vQNGk | | wHseh | |

### REFERENCES

1. E. M. Driggers, S. P. Hale, J. Lee and N. K. Terrett, Nat. Rev. Drug Discov., 2008, 7, 608-624.
2. M. R. Naylor, A. T. Bockus, M. J. Blanco and R. S. Lokey, Curr. Opin. Chem. Biol., 2017, 38, 141-147.
3. D. S. Nielsen, N. E. Shepherd, W. Xu, A. J. Lucke, M. J. Stoermer and D. P. Fairlie, Chem. Rev., 2017, 117, 8094-8128.
4. J. Witek, B. G. Keller, M. Blatter, A. Meissner, T. Wagner and S. Riniker, J. Chem. Inf. Model., 2016, 56, 1547-1562.
5. J. Witek, M. Muhlbauer, B. G. Keller, M. Blatter, A. Meissner, T. Wagner and S. Riniker, Chemphyschem, 2017, 18, 3309-3314.
6. J. Witek, S. Wang, B. Schroeder, R. Lingwood, A. Dounas, H. J. Roth, M. Fouche, M. Blatter, O. Lemke, B. Keller and S. Riniker, J. Chem. Inf. Model., 2019, 59, 294-308.
7. S. Ono, M. R. Naylor, C. E. Townsend, C. Okumura, O. Okada and R. S. Lokey, J. Chem. Inf. Model., 2019, 59, 2952-2963.
8. A. Liwo, A. Tempczyk, S. Oldziej, M. D. Shenderovich, V. J. Hruby, S. Talluri, J. Ciarkowski, F. Kasprzykowski, L. Lankiewicz and Z. Grzonka, Biopolymers, 1996, 38, 157-175.
9. E. Haensele, L. Banting, D. C. Whitley and T. Clark, J. Mol. Model., 2014, 20, 2485.
10. E. Yedvabny, P. S. Nerenberg, C. So and T. Head-Gordon, J. Phys. Chem. B, 2015, 119, 896-905.
11. E. Haensele, N. Saleh, C. M. Read, L. Banting, D. C. Whitley and T. Clark, J. Chem. Inf. Model., 2016, 56, 1798-1807.
12. A. Zorzi, K. Deyle and C. Heinis, Curr. Opin. Chem. Biol., 2017, 38, 24-29.
13. D. S. Wishart, Y. D. Feunang, A. C. Guo, E. J. Lo, A. Marcu, J. R. Grant, T. Sajed, D. Johnson, C. Li, Z. Sayeeda, N. Assempour, I. lynkkaran, Y. Liu, A. Maciejewski, N. Gale, A. Wilson, L. Chin, R. Cummings, D. Le, A. Pon, C. Knox and M. Wilson, Nucleic Acids Res., 2018, 46, D1074-D1082.
14. X. Jing and K. Jin, Med. Res. Rev., 2020, 40, 753-810.
15. T. Rezai, J. E. Bock, M. V. Zhou, C. Kalyanaraman, R. S. Lokey and M. P. Jacobson, J. Am. Chem. Soc., 2006, 128, 14073-14080.
16. A. Whitty, M. Zhong, L. Viarengo, D. Beglov, D. R. Hall and S. Vajda, Drug Discov. Today, 2016, 21, 712-717.
17. P. G. Dougherty, A. Sahni and D. Pei, Chem. Rev., 2019, 119, 10241-10287.
18. B. Over, P. Matsson, C. Tyrchan, P. Artursson, B. C. Doak, M. A. Foley, C. Hilgendorf, S. E. Johnston, M. D. t. Lee, R. J. Lewis, P. McCarren, G. Muncipinto, U. Norinder, M. W. Perry, J. R. Duvall and J. Kihlberg, Nat. Chem. Biol., 2016, 12, 1065-1074.
19. D. D. Boehr, R. Nussinov and P. E. Wright, Nat. Chem. Biol., 2009, 5, 789-796.
20. I. J. Chen and N. Foloppe, Bioorg. Med. Chem., 2013, 21, 7898-7920.
21. V. Poongavanam, E. Danelius, S. Peintner, L. Alcaraz, G. Caron, M. D. Cummings, S. Wlodek, M. Erdelyi, P. C. D. Hawkins, G. Ermondi and J. Kihlberg, ACS Omega, 2018, 3, 11742-11757.
22. V. Poongavanam, Y. Atilaw, S. Ye, L. H. E. Wieske, M. Erdelyi, G. Ermondi, G. Caron and J. Kihlberg, J. Pharm. Sci., 2021, 110, 301-313.
23. P. Hosseinzadeh, G. Bhardwaj, V. K. Mulligan, M. D. Shortridge, T. W. Craven, F. Pardo-Avila, S. A. Rettie, D. E. Kim, D. A. Silva, Y. M. Ibrahim, I. K. Webb, J. R. Cort, J. N. Adkins, G. Varani and D. Baker, Science, 2017, 358, 1461-1466.
24. D. P. Slough, S. M. McHugh, A. E. Cummings, P. Dai, B. L. Pentelute, J. A. Kritzer and Y. S. Lin, J. Phys. Chem. B, 2018, 122, 3908-3919.
25. N. el Tayar, A. E. Mark, P. Vallat, R. M. Brunne, B. Testa and W. F. van Gunsteren, J. Med. Chem., 1993, 36, 3757-3764.
26. H. Morita, Y. S. Yun, K. Takeya, H. Itokawa and M. Shiro, Tetrahedron, 1995, 51, 5987-6002.
27. Y. Chen, K. Deng, X. Qiu and C. Wang, Sci. Rep., 2013, 3, 2461.
28. C. Merten, F. Li, K. Bravo-Rodriguez, E. Sanchez-Garcia, Y. Xu and W. Sander, Phys. Chem. Chem. Phys., 2014, 16, 5627-5633.
29. J. S. Quartararo, M. R. Eshelman, L. Peraro, H. Yu, J. D. Baleja, Y. S. Lin and J. A. Kritzer, Bioorg. Med. Chem., 2014, 22, 6387-6391.
30. D. P. Slough, S. M. McHugh and Y. S. Lin, Biopolymers, 2018, 109, e23113.
31. S. M. McHugh, J. R. Rogers, H. Yu and Y. S. Lin, J. Chem. Theory Comput., 2016, 12, 2480-2488.
32. A. Laio and M. Parrinello, Proc. Natl. Acad. Sci. U.S.A., 2002, 99, 12562-12566.
33. S. Piana and A. Laio, J. Phys. Chem. B, 2007, 111, 4553-4559.
34. S. M. McHugh, H. Yu, D. P. Slough and Y. S. Lin, Phys. Chem. Chem. Phys., 2017, 19, 3315-3324.
35. A. E. Cummings, J. Miao, D. P. Slough, S. M. McHugh, J. A. Kritzer and Y. S. Lin, Biophys. J., 2019, 116, 433-444.
36. V. Hornak, R. Abel, A. Okur, B. Strockbine, A. Roitberg and C. Simmerling, Proteins, 2006, 65, 712-725.
37. C. Y. Zhou, F. Jiang and Y. D. Wu, J. Phys. Chem. B, 2015, 119, 1035-1047.
38. W. L. Jorgensen, J. Chandrasekhar, J. D. Madura, R. W. Impey and M. L. Klein, J. Chem. Phys., 1983, 79, 926-935.
39. H. Geng, F. Jiang and Y. D. Wu, J. Phys. Chem. Lett., 2016, 7, 1805-1810.
40. A. Yousef and N. M. Charkari, J. Biomed. Inform., 2015, 56, 300-306.
41. H. L. Morgan, J. Chem. Doc., 1965, 5, 107-113.
42. D. Rogers and M. Hahn, J. Chem. Inf. Model., 2010, 50, 742-754.
43. A. Rodriguez and A. Laio, Science, 2014, 344, 1492-1496.

1. A method for predicting a structure of a cyclic peptide, the method comprising providing a weight vector w, wherein w comprises a multiplicity residue weights of an adopted structure and a multiplicity of partition function weights,
providing a coefficient matrix A configured to select which of the multiplicity residue weights of the adopted structure and which one of the multiplicity of partition function weights are used to determine the population of a cyclic peptide adopting the structure, and
determining the population of the structure of the cyclic peptide from the multiplicity of residue weights and multiplicity of partition function weights.

2. The method of claim 1, wherein the multiplicity of residue weights of the adopted structure and the multiplicity of partition function weights are determined by minimizing the difference between a predicted population and an actual population observed in a training dataset.

3. The method of claim 2, wherein the training dataset is obtained from a molecular dynamics simulation.

4. The method of claim 1, wherein the multiplicity of residue weights are a multiplicity of pairwise (1, 2) residue weights, (1, 3) residue weights, (1, 4) residue weights, or any combination thereof.

5. The method of claim 4, wherein the multiplicity of pairwise residue weights of the adopted structure and the multiplicity of partition function weights are determined by minimizing the difference between a predicted population and an actual population observed in a training dataset.

6. The method of claim 5, wherein the training dataset is obtained from a molecular dynamics simulation.

7. A method for predicting a population of a structure of a cyclic peptide, the method comprising encoding the cyclic peptide and determining the population of the structure of the cyclic peptide with a neural network.

8. The method of claim 7, wherein the cyclic peptide is encoded with a molecular fingerprint encoding scheme.

9. The method of claim 7, further comprising representing a cyclic peptide as a graph with a node for every amino acid of the cyclic peptide and connecting a node pair by forward and backward edges, wherein the initial node representation is given by an amino acid molecular fingerprint.

10. The method of claim 9, wherein the neural network is a graph neural network.

11. The method of claim 7, further comprising arranging an initial representation of the cyclic peptide such that neighboring amino acids have features adjacent in space.

12. The method of claim 11, wherein the neural network is a convolutional neural network.

13. The method of claim 7, wherein the neural network is trained with a training dataset is obtained from a molecular dynamics simulation.

14. A method for selecting a cyclic peptide, the method comprising performing the method according to claim 1 for a plurality of different cyclic peptides and selecting well-structured cyclic peptides from the plurality of different cyclic peptides.

15. The method of claim 14, further comprising synthesizing one or more of the selected cyclic peptide.

16. The method of claim 15, wherein the method comprises assaying the synthesized cyclic peptide selected cyclic peptide.

17. The method of claim 14, wherein the method comprises assaying one or more of the selected cyclic peptides.

18. A computational platform comprising:
a communication interface that receives cyclic peptide information, and
a computer in communication with the communication interface, wherein the computer comprises a computer processor and a computer readable medium comprising machine-executable code that, upon execution by the computer processor, implements the method according to claim 1 for the cyclic peptide.

19. The computational platform of claim 18, wherein the method further comprises generating a report of well-structured cyclic peptides.

20. A computer readable medium comprising machine-executable code that, upon execution by the computer processor, implements the method according to claim 1.

21. The computer readable medium of claim 20, wherein the method further comprises generating a report of well-structured cyclic peptides.
